(19) United States
(12) Patent Application Publication
(10) Pub. No.: US 2024/0316050 A1
(43) Pub. Date: Sep. 26, 2024

(54) TREATMENT OF DISORDERS ASSOCIATED WITH LOW BH4 BIOAVAILABILITY

(71) Applicant: OXFORD UNIVERSITY INNOVATION LIMITED, Oxford (GB)

(72) Inventor: Keith CHANNON, Oxford (GB)

(73) Assignee: OXFORD UNIVERSITY INNOVATION LIMITED, Oxford (GB)

(21) Appl. No.: 18/266,179

(22) PCT Filed: Dec. 21, 2021
(86) PCT No.: PCT/GB2021/053382

§ 371 Date: Jun. 8, 2023

(30) Foreign Application Priority Data
Dec. 21, 2020 (GB) 2020285.9
Jun. 9, 2021 (GB) 2108231.8

(51) Int. Cl. A61K 31/519; A61P 9/12; G01N 33/573

(52) U.S. Cl. CPC A61K 31/519; A61P 9/12; G01N 33/5735; G01N 2800/52

(57) ABSTRACT

The present invention relates to the use of a reduced folate, such as 5-MTHF (5-methyltetrahydrofolate), optionally in combination with tetrahydrobiopterin (BH4) or precursors thereof, for preventing or treating disorders associated with a BH4 deficiency, or low BH4 bioavailability. Such disorders primarily include diseases which have vascular endothelial pathology, or effects on amino acid metabolism or neurotransmission, especially pregnancy related disorders such as pre-eclampsia.

### FIELD OF THE INVENTION

The present invention relates to the use of a reduced folate, preferably 5-MTHF (5-methyltetrahydrofolate), optionally in combination with tetrahydrobiopterin (BH4) or precursors thereof, for preventing or treating disorders associated with a BH4 deficiency, or low BH4 bioavailability, which may occur systemically, or locally in specific cells or tissues.

Such disorders primarily include diseases which have vascular endothelial pathology, or effects on amino acid metabolism or neurotransmission. The invention further relates to methods of selecting patients for treatment with said reduced folate, optionally in combination with BH4 or precursors thereof, compositions and kits comprising a reduced folate and BH4, a precursor or functional equivalent thereof, kits, and in vitro uses of a reduced folate, preferably 5-MTHF, in preventing the oxidation of BH4 or precursors thereof.

### BACKGROUND OF THE INVENTION

Endothelial dysfunction is associated with the markers of reduced vascular nitric oxide (NO) bioavailability and oxidative stress. Lack of nitric oxide (NO) is known to be a factor in many diseases involving the vascular endothelium, such as cardiovascular diseases, or disorders of pregnancy such as pregnancy-induced hypertension, placental insufficiency, foetal growth restriction and pre-eclampsia.

During pregnancy, vascular remodelling is a requirement for normal development, in order to provide adequate blood flow for placental perfusion that ensures fetal growth. Vascular adaptation in pregnancy requires remodelling of the uterine arteries and development of the placental vasculature sufficient to accommodate a major increase in uterine blood flow, without causing an increase in systemic blood pressure. In normal pregnancy, increased uterine artery calibre is associated with enhanced activity of endothelial nitric oxide synthase (eNOS), and nitric oxide (NO) bioavailability.

Inadequate uteroplacental vascular remodelling to cope with this increased blood flow causes pregnancy-induced hypertension and fetal growth restriction, or pre-eclampsia. These are major causes of adverse pregnancy outcomes for both mother and child, affecting ˜7% of all pregnancies world-wide. Nearly one-tenth of all maternal deaths in Africa and Asia and one-quarter in Latin America are associated with hypertensive diseases in pregnancy, including pre-eclampsia. In these conditions, uterine and placental blood vessels show increased medial vascular smooth muscle cell (VSMC) hypertrophy and reduced calibre. NO-dependent flow-mediated vasodilatation is further reduced in arteries isolated from pre-eclamptic pregnancies. This results in placental insufficiency, associated with increased plasma biomarkers related to endothelial cell dysfunction and abnormal angiogenesis, such as placental growth factor (PIGF)(4), sEng, and sFLT-1. Such endothelial dysfunction is a consistent finding in many pregnancy-related disorders.

Tetrahydrobiopterin (BH4) is a redox cofactor for endothelial nitric oxide synthase (eNOS) with a required role in NO generation. A common feature of abnormal eNOS activity in other cardiovascular diseases, is loss of the required eNOS cofactor tetrahydrobiopterin (BH4), which is synthesised in endothelial cells by the enzyme GTP cyclohydrolase I, encoded by the GCH1 gene. However, BH4 has not been investigated as a potential therapeutic target in the treatment of pregnancy related disorders such as pre-eclampsia.

Therapies for pregnancy related disorders stemming from vascular endothelial pathology have thus far focused on treating the symptoms of the condition, typically by lowering blood pressure, until the fetus can be delivered. Pre-eclampsia rarely happens before the 20^{th} week of pregnancy. Most cases occur after 24 to 26 weeks, and usually towards the end of pregnancy. Although less common, the condition can also develop for the first time in the first 6 weeks after birth (=postpartum preeclampsia). Most people only experience mild symptoms, but it's important to manage the condition in case severe symptoms or complications develop. Generally, the earlier pre-eclampsia develops, the more severe the condition will be. The definitive treatment for pre-eclampsia, for example, is the delivery of the fetus and placenta. The primary medications used to lower blood pressure prior to delivery are aspirin (for mild cases), labetalol, nifedipine or methyldopa. Only one of which, labelatol, is licensed for use in pregnant women in the UK. It is difficult to develop medications and obtain approval for treatment of pregnant women given the primary concern of safety of the fetus. Anti-convulsant medication such as magnesium sulfate is also sometimes administered to prevent seizures. Even with such treatments, given the risks involved with such conditions, many mothers are forced to stay in hospital for close monitoring until the fetus is delivered, or undergo preterm delivery.

There are no proven preventative therapies available for such pregnancy-related disorders beyond taking low-dose aspirin. However this is only effective in helping to prevent high blood pressure. It does not address the other effects of these conditions, such as poor placental formation and development, and hypoxia. There are currently no therapies which solve the underlying vascular causes of these conditions.

In addition to its function in endothelial NO generation, Tetrahydrobiopterin (BH4) is a redox cofactor for other enzymes, specifically a group of amino acid hydroxylases which convert the amino acids tryptophan, phenylalanine and tyrosine. These enzymes are used in the degradation of the amino acid phenylalanine and in the biosynthesis of the neurotransmitters serotonin (5-hydroxytryptamine, 5 HT), melatonin, dopamine, norepinephrine (noradrenaline), and epinephrine (adrenaline).

Numerous disorders are connected with dysfunction of these hydroxylase enzymes which can also be caused by a deficiency, or reduction in the bioavailability of, the required cofactor BH4. Such disorders include, for example, BH4 deficiency itself and phenylketonuria (PKU). In addition, due to its role in neurotransmitter production, BH4 is also implicated in neurological disorders such as autism, ADHD, depression, and Parkinson's. Low availability of BH4 in specific cells and tissues may not be related only to systemic BH4 levels, but may also be influenced by local BH4 oxidation or recycling, and the distribution of BH4, for example by cellular uptake or transport.

However, as with the treatment of cardiovascular and pregnancy-related disorders, many of these diseases rely on other therapeutics which are not based on the involvement of BH4.

Only one commercially available therapeutic comprising BH4 is available in the form of sapropterin dihydrochloride (BH4*2HCL) and is approved for use in the treatment of PKU. However, most people with PKU have little or no benefit from the drug (Camp K M, Parisi M A, Acosta P B, Berry G T, Bilder D A, Blau N, et al. (June 2014) Molecular Genetics and Metabolism. 112 (2): 87-122). Furthermore, administration of BH4 in an oral form had no benefit in patients with existing vascular disease and endothelial dysfunction (Cunnington C et al. Circulation 2012). Another significant problem with using BH4 is that it is susceptible to oxidation.

The present invention is intended to solve one or more of the above-mentioned problems by one or more of the following aspects.

### STATEMENTS OF INVENTION

According to a first aspect of the present invention, there is provided a reduced folate for use in the prevention or treatment of a disorder associated with low BH4 bioavailability, wherein the reduced folate is not for administration in combination with any of the following:
aspirin;
a herbal extract.

In one embodiment, the reduced folate is for administration in combination with BH4, a precursor, or functional equivalent thereof.

In one embodiment, the reduced folate, and optionally BH4, a precursor, or functional equivalent thereof, are the sole active pharmaceutical agents. In one embodiment, the reduced folate is the sole active pharmaceutical agent.

According to a second aspect of the present invention, there is provided a combination of a reduced folate and BH4, a precursor, or functional equivalent thereof for use in the prevention or treatment of a disorder associated with low BH4 bioavailability.

According to a third aspect of the present invention, there is provided a composition comprising one or more active pharmaceutical ingredients for use in the prevention or treatment of a disorder associated with low BH4 bioavailability, wherein the active pharmaceutical ingredients consist of a reduced folate and optionally BH4, a precursor, or functional equivalent thereof.

In one embodiment, the active pharmaceutical ingredient consists of a reduced folate.

According to a fourth aspect of the present invention, there is provided a method of treating a subject having a disorder associated with low BH4 bioavailability, the method comprising administering to said subject a reduced folate, wherein the reduced folate is not administered in combination with any of the following:
aspirin;
a herbal extract.

In one embodiment, the reduced folate is administered in combination with BH4, a precursor, or functional equivalent thereof.

In one embodiment, the reduced folate, and optionally BH4, a precursor, or functional equivalent thereof, are the sole active pharmaceutical agents. In one embodiment, the reduced folate is the sole active pharmaceutical agent.

According to a fifth aspect of the present invention, there is provided a method of treating a subject having a disorder associated with a low BH4 bioavailability, the method comprising administering to said subject a reduced folate and BH4, a precursor, or functional equivalent thereof.

According to a sixth aspect of the present invention, there is provided a method of treating a subject having a disorder associated with low BH4 bioavailability, the method comprising administering to said subject a composition comprising one or more active pharmaceutical ingredients, wherein the active pharmaceutical ingredients consist of a reduced folate and optionally BH4, a precursor, or functional equivalent thereof.

In one embodiment, the active pharmaceutical ingredient consists of a reduced folate.

In one embodiment of any of the fourth, fifth or sixth aspects, the method further comprises determining the level of one or more markers of a disorder associated with low BH4 bioavailability in the subject, and suitably comparing the or each marker level to a reference level of a healthy subject. In one embodiment, the method further comprises selecting the subject for treatment if the subject exhibits one or more markers of a disorder associated with low BH4 bioavailability.

According to a seventh aspect of the present invention, there is provided use of a reduced folate in the manufacture of a medicament for the treatment of a disorder associated with low BH4 bioavailability, wherein the medicament does not comprise any of the following:
aspirin;
a herbal extract.

In one embodiment, the reduced folate is used in combination with BH4, a precursor, or functional equivalent thereof in the manufacture of the medicament.

In one embodiment, the reduced folate, and optionally BH4, a precursor, or functional equivalent thereof, are the sole active pharmaceutical agents used in the manufacture of the medicament. In one embodiment, the reduced folate is the sole active pharmaceutical agent used in the manufacture of the medicament.

According to an eighth aspect of the present invention, there is provided use of a reduced folate in combination with BH4, a precursor, or functional equivalent thereof in the manufacture of a medicament for the treatment of a disorder associated with low BH4 bioavailability.

According to an ninth aspect of the present invention, there is provided use of one or more active pharmaceutical ingredients in the manufacture of a medicament for the treatment of a disorder associated with low BH4 bioavailability, wherein the active pharmaceutical ingredients consist of a reduced folate and optionally BH4, a precursor, or functional equivalent thereof.

In one embodiment, the active pharmaceutical ingredient consists of a reduced folate.

In one embodiment of any of the above aspects, the disorder associated with low BH4 bioavailability is a pregnancy-related disorder.

In one embodiment of any of the above aspects, the reduced folate normalises or restores BH4 levels. In one embodiment of any of the above aspects, the reduced folate increases BH4 levels. In one embodiment, the BH4 levels are measured in endothelial cells.

In one embodiment of any of the above aspects, the reduced folate reduces or prevents oxidation of BH4.

In one embodiment of any of the above aspects, the prevention or treatment is of a subject, suitably a subject having one or more markers of a disorder associated with low BH4 bioavailability. In one embodiment, the subject has been tested for one or more markers of a disorder associated with low BH4 bioavailability, suitably by the method of the tenth aspect.

According to a tenth aspect of the present invention, there is provided a method of selecting a subject that may benefit from treatment with a reduced folate and optionally BH4, a precursor or functional equivalent thereof, the method comprising determining the level of one or more markers of a disorder associated with low BH4 bioavailability in a subject, comparing the level(s) of the or each marker to a reference level(s) in a healthy subject, and selecting the subject for treatment if the subject exhibits an abnormal level of the one or more markers of a disorder associated with low BH4 bioavailability compared to the reference level.

Suitable markers of a disorder associated with low BH4 bioavailability are described below. Suitably, the term ‘abnormal’ means that the level of the marker is significantly different from that in a healthy subject. Suitably an ‘abnormal’ level of a marker may be increased or decreased relative to that in a healthy subject.

In one embodiment of the tenth aspect, the one or more markers of disorder associated with low BH4 bioavailability comprise at least the BH4 level in a subject. Suitably in such an embodiment, the method comprises determining the BH4 level in a subject, and optionally determining the level of one or more further markers of a disorder associated with low BH4 bioavailability in the subject.

In one embodiment of the tenth aspect, the method further comprises providing a treatment to the selected subject. In one embodiment, the treatment comprises a reduced folate and optionally BH4, a precursor, or functional equivalent thereof.

According to an eleventh aspect of the present invention there is provided a pharmaceutical composition comprising a reduced folate and BH4, a precursor, or functional equivalent thereof.

According to a twelfth aspect of the present invention, there is provided a kit comprising:
a reduced folate; and
BH4, a precursor, or functional equivalent thereof.

According to a thirteenth aspect of the present invention, there is provided use of a reduced folate to prevent oxidation of BH4, a precursor, or a functional equivalent thereof, in vitro.

In one embodiment of the thirteenth aspect, the reduced folate may be in its natural or unnatural stereoisomeric form.

In one embodiment of any of the above aspects, the reduced folate is in its natural stereoisomeric form. Suitably, in aspects relating to medical uses, the reduced folate is in its natural stereoisomeric form.

In one embodiment of any of the above aspects, the reduced folate comprises a fully reduced folate such as 5-MTHF.

In one embodiment of any of the above aspects, low BH4 bioavailability refers to a reduction in BH4 bioavailability, suitably a low level of BH4 relative to the level of BH4 in a healthy subject. Suitably low BH4 bioavailability comprises systemic low BH4 bioavailability, or local low BH4 bioavailability. Suitably local low BH4 bioavailability may comprise a reduction in BH4 at the tissue level or cellular level, for example low tissue BH4 bioavailability or low cellular BH4 bioavailability in specific tissues or cells. Suitably low BH4 bioavailability may result from a BH4 deficiency. Alternatively or additionally, low BH4 bioavailability may result from impaired BH4 transport or BH4 uptake into cells, or increased oxidation or reduction of BH4, for example.

The present inventors have discovered the mechanism by which BH4 is involved in the pathology of pregnancy related vascular disorders such as pre-eclampsia, and potentially many other disorders relating to vascular endothelial pathology, such as cardiovascular, liver and kidney diseases.

The inventors have further discovered an effective therapy to increase BH4 levels, which could be applied to any disorder which is mediated by low BH4 bioavailability.

In relation to pregnancy related vascular disorders, the inventors have found that loss of BH4 mediates maternal endothelial dysfunction and is the primary contributor to pregnancy-related vascular pathogenesis. The inventors have surprisingly found that the lack of BH4 in endothelial cells is a key cause of such conditions, making it a viable therapeutic target.

The inventors have found that these conditions are associated with reduced endothelial cell BH4 levels, impaired eNOS activity, and reduced endothelial cell proliferation, mediated by reduced GTP cyclohydrolase I protein, the rate limiting enzyme in BH4 biosynthesis. Working in pregnant mice, the inventors have shown that maternal endothelial cell BH4 deficiency, resulting from targeted deletion of the Gch1 gene (encoding the synthesis enzyme GTP cyclohydrolase 1), caused progressive hypertension during pregnancy and fetal growth restriction. Furthermore the inventors have shown that maternal endothelial cell Gch1 deletion causes defective functional and structural remodelling in uterine arteries and in spiral arteries, leading to placental insufficiency.

Prior to the inventor's work, the mechanisms of NO-mediated vascular remodelling in pregnancy-related vascular disorders had not been elucidated. It was not known that BH4 was involved in the pathology of such conditions, nor was it known that BH4 had such an important role in the vascular remodelling required for normal placental development. The inventors have identified a critical requirement for maternal endothelial cell BH4 biosynthesis in uteroplacental vascular remodelling during pregnancy.

Prior attempts to treat other disorders involving BH4 such as BH4 deficiency itself or phenylketonuria (PKU) have involved treating patients directly with oral BH4 tablets.

However, the inventors have further found that disorders involving BH4 cannot always be treated by providing BH4 itself as a medicament. The inventors have discovered that BH4 is rapidly oxidised to the non-effective form; BH2. High-blood pressure and fetal growth restriction in pregnant endothelial cell Gch1 deficient mice was not rescued by oral BH4 supplementation, due to systemic oxidation of BH4 to BH2. Such rapid oxidation to BH2 also occurred in control mice, indicating that direct BH4 supplementation is not effective to treat disorders associated with low BH4 bioavailability.

In order to solve this problem, the inventors have found that providing BH4 as a combination therapy together with a reduced folate solves this issue, and retains BH4 in its effective reduced state. Providing the fully reduced folate, 5-methyltetrahydrofolate (5-MTHF) prevented BH4 oxidation, reduced blood pressure to normal levels and normalized fetal growth.

Surprisingly, the effect of the reduced folate has also been discovered to work on existing BH4 present in the body, allowing the reduced folate to be used as a medicament alone.

Whilst the related chemical folic acid, a fully oxidized form of folate, has long been recommended as a supplement for pregnant women, clinical trials have shown no benefit of folic acid supplementation in women with pre-eclampsia. Indeed, in the inventor's own work, folic acid does not show any therapeutic effect when provided to Gch1 deficient mice. Folic acid requires conversion to 5-MTHF by chemical reduction, so is unable to exert the beneficial redox effects on BH4 levels that the inventors have observed with the fully reduced form of 5-MTHF. The novel effect of 5-MTHF in augmenting BH4 availability in cells and preventing BH4 oxidation that has been discovered by the inventors, is surprisingly independent of any prior known effects of folate mediated classical 1-carbon folate pathways, or homocysteine lowering.

Surprisingly therefore the inventors have found that provision of a medicament where the active pharmaceutical ingredients consist of a reduced folate and optionally BH4 is effective to prevent and treat pregnancy-related vascular disorders. Restoration of endothelial cell BH4 with a reduced folate provides an effective therapy to intervene in pregnancy-related hypertension, placental insufficiency, and associated fetal growth restriction. Advantageously, this therapy can specifically be used to treat or prevent pre-eclampsia, which has no current effective treatment, by dealing with the underlying endothelial pathology, rather than merely alleviating the hypertensive symptoms thereof. Prevention of pre-eclampsia can start any time before 20 weeks after gestation, preferably before 16 weeks after gestation, even more preferably before 12 weeks after gestation and last for the entire period of pregnancy plus 12 weeks after birth.

Even more surprisingly the inventors have found that provision of a medicament where the active pharmaceutical ingredients consist of a reduced folate and optionally BH4 is effective to prevent recurring and inherited pregnancy-related vascular disorders.

BH4 is also a key component of other mechanisms in the body. Whilst BH4 is a cofactor for the production of nitric oxide (NO) by nitric oxide synthase, which may explain its involvement in causing endothelial dysfunction such as in vascular disorders including pre-eclampsia, BH4 is also a cofactor for other enzymes, for example the group of aromatic amino acid hydroxylase enzymes that convert amino acids in to neurotransmitters, or catabolise amino acids such as phenylalanine. The inventors further expect that restoration or augmentation of BH4 availability within other cell or tissue types will provide beneficial effects for the treatment of other diseases where dysfunction of these enzymes is known to play a key role. For example in BH4 deficiency itself, PKU, or neurological disorders such as Parkinson's disease, autism and ADHD. Advantageously, the discovery of a different therapeutic which does not rely on provision of BH4 itself to have a beneficial effect could be of great use in these disorders. This avoids the issues with using BH4 alone as a therapeutic, which may be more costly and challenging to formulate and administer, and which studies have shown may not actually provide any beneficial effects in vivo due to rapid oxidation in to an ineffective form.

### FIGURES

The invention will now be described with reference to the following figures in which:

FIG. 1 shows: GTPCH protein, BH4 levels, NOS activity and in vitro endothelial tube formation in endothelial cells from normotensive and pre-eclamptic pregnancies. Human umbilical vein endothelial cells (HUVECs) were isolated from umbilical cords of placentas from either normotensive (NT) or pre-eclamptic (PE) pregnancies. HUVECs were cultured in endothelial cell growth medium and harvested for analysis of biopterins, GTPCH protein levels and NOS activity. (a-b) HPLC analysis of biopterins in HUVECs from normotensive (NT) and pre-eclamptic pregnancies (PE). Biopterins are expressed per mg of cellular protein (*P<0.05; n=12 to 14 per group); (c) Representative immunoblots showing GTPCH protein in HUVECs from NP and PE pregnancies, with band density quantified relative to β-tubulin loading control (*P<0.05; n=9 per group); (d) Endothelial nitric oxide synthase activity (eNOS) was measured by conversion of 14C arginine in cell culture, followed by radiochemical HPLC quantification of 14C citrulline production. eNOS activity was greatly reduced in HUVECs from PE compared to NT under basal conditions and when stimulated with calcium ionophore (A23187). Treatment with sepiapterin (Sep; 1 μM, converted to BH4 by the cellular pterin salvage pathway) significantly increased eNOS activity in both NT and PE HUVECs, such that eNOS activity was no longer different between the groups. The non-selective NOS inhibitor NG-methyl-L-arginine (L-NMA), abolished eNOS activity in all groups (*P<0.05 n=6-8 per group); (e) The levels of BH4 in HUVECs from NT and PE treated with or without 1 μM sepiapterin; (f) Representative photomicrographs of HUVECs from NT and PE plated on growth factor-reduced matrigel in the presence or absence of sepiapterin (1 μM); (g) Quantification of endothelial branches points and total tube length was performed using Angiosys software and expressed in micrometres per field. HUVECs from PE showed lesser endothelial cell growth and tubule formation than HUVECs from NT, but were rescued by supplementation with sepiapterin (*P<0.05 n=6 per group); (g) Quantification of endothelial branches points and total tube length was performed using Angiosys software and expressed in micrometres per field. HUVECs from PE showed lesser endothelial cell growth and tubule formation than HUVECs from NT, but were rescued by supplementation with sepiapterin (*P<0.05 n=6 per group); (h) Extracellular vesicles were isolated by dual-lobe placental perfusion and ultracentrifugation from placentas from women with normotensive pregnancies (NT) or from women with pre-eclampsia (PE). The levels of BH4, BH2 and total biopterins (i.e. BH4+BH2+biopterin (B)) and ratio of BH4 relative to oxidized biopterin species “BH4/total biopterin ratio” (i.e. BH4/(BH2+B)) were measured by HPLC. BH4, total biopterins and BH4/total biopterin ratio were significantly reduced in extracellular vesicles isolated from women with pre-eclamptic pregnancies compared to women with normotensive pregnancies (*P<0.05; n=6 per group).

FIG. 2 shows: Effect of Gch1 knockdown on in vitro endothelial tube formation in endothelial cells. sEnd.1 murine endothelial cells (a-e) and primary human uterine microvascular endothelial cells (HutMECS) (f-h) were transfected with an siRNA pool targeted to Gch1 or a nontargeting (NS) scrambled control siRNA. The cells were then harvested and analysed for GTPCH protein expression by Western blotting, or biopterin levels using HPLC with electrochemical and fluorescent detection. (a) Representative Western blot for GTPCH protein in sEND.1 mouse endothelial cell line treated with non-specific Gch1 siRNA (NS) or specific Gch1 siRNA (Gch1 siRNA). In sEND.1 treated with specific Gch1 siRNA, GTPCH protein was not detectable by Western blotting, whereas GTPCH was readily detected in sEND.1 treated with non-specific Gch1 siRNA. The blot is representative of four separate experiments; (b) Intracellular BH4, BH2, total biopterins (i.e. BH4+BH2+B) and ratio of BH4 relative to oxidized biopterin species “BH4/total biopterin ratio” (i.e. BH4/(BH2+B)), measured by HPLC, were significantly reduced in Gch1 specific siRNA cells compared with non-specific siRNA cells (*P<0.05 n=4 per group); (c) Representative photomicrographs of sEND.1 mouse endothelial cell treated with nonspecific Gch1 siRNA or specific Gch1 siRNA plated on growth factor-reduced matrigel in the presence or absence of sepiapterin (1 μM). Quantification of endothelial tube length and branching was performed by using Angiosys software and expressed in micrometres per field; (d) In vitro endothelial tube formation and branching. Reduction of Gch1 expression in sEND.1 treated with specific Gch1 siRNA caused a marked decrease in endothelial cell growth and tubule formation compared to sEND.1 treated with non-specific Gch1 siRNA, which can be rescued by supplementation with sepiapterin (*P<0.05 n=6 per group); (e) The levels of BH4 in sEND.1 cells treated with non-specific Gch1 siRNA or specific Gch1 siRNA in the presence or absence of sepiapterin (1 μM)(*P<0.05 n=6 per group); (f) Western blot analysis shows that cellular GTPCH protein was greatly reduced in HutMECs following exposure to GCH1-specific siRNA, compared to non-specific (NS) siRNA. The identity of bands obtained were confirmed using control lysates from HUVECs treated with siRNA GCH1 (as negative control) or with non-specific siRNA (as positive control); (g) GCH1 specific siRNA significantly reduced the detectable levels of the cellular biopterins and the ratio of BH4 to BH2+B (BH4/total biopterins ratio) (*P<0.05 n=4 per group); (h) Representative photomicrographs of HutMECs treated with non-specific GCH1 siRNA or specific GCH1 siRNA plated on growth factor-reduced matrigel in the presence or absence of sepiapterin (1 μM). Quantification of endothelial tube length and branching was performed by using Angiosys software and expressed in micrometres per field; (i) In vitro endothelial tube formation and branching. Reduction of GCH1 expression in human uterine endothelial cells caused to a significant decrease in endothelial cell growth and tubule formation, which can rescued by supplementation with sepiapterin (1 μM)(*P<0.05 n=4 per group).

FIG. 3 shows: Effects of endothelial cell-specific Gch1 knockout in pregnant mice. Pregnancy was achieved by mating virgin wild-type female mice and Gch1^{fl/fl}Tie2cre female mice (aged between 10-16 weeks old) with wild-type male mice. Tissues were harvested and collected for experiments at E18.5 day of gestation, or from non-pregnant mice. (a-d) Levels of biopterins in aortas, uterine arteries and plasma from non-pregnant mice and pregnant (E18.5 day gestation) wild-type (i.e. Gch1^{fl/fl}) and Gch1^{fl/fl}Tie2cre mice were measured by HPLC. The BH4 and total biopterin levels were significantly decreased in Gch1^{fl/fl}Tie2cre mice compared to wild-type mice in both non-pregnant and pregnant mice. In non-pregnant mice, there was no significantly difference in plasma BH4 between wildtype and Gch1^{fl/fl}Tie2cre mice. In pregnant mice, plasma BH4 levels were significantly reduced in both wild-type and Gch1^{fl/fl}Tie2cre mice but with a greater extent in Gch1^{fl/fl}Tie2cre mice such that the BH4/(BH2+B) ratio was significantly decreased in Gch1^{fl/fl}Tie2cre mice. The open (white) bars in each case are the levels of BH4, the grey filled bars are the total biopterins (i.e. BH4+BH2+B). (*P<0.05 n=7-10 animals per group); (e) Systolic blood pressure was measured by non-invasive tail-cuff plethysmography in wildtype (Gch1^{fl/fl}) and Gch1^{fl/fl}Tie2cre mice before and during pregnancy. (t P<0.05 comparing genotype; *P<0.05 comparing baseline blood pressure; n=7 to 10 animals per group); (f) Increased maternal weight gain during pregnancy was reduced in Gch1^{fl/fl}Tie2cre mice compared to wild-type mice. (*P<0.05 n=7-10 animals per group); (g and h) The number of fetuses per litter (litter size) or parturition day between wild-type and Gch1^{fl/fl}Tie2cre mice; (i-k) Fetuses from wild-type and Gch1^{fl/fl}Tie2cre mothers were collected and weighed at E18.5 day of gestation (*P<0.05 n=72 to 85 pups from 10 to 13 litters per group); (I and m) Offspring weights from wild-type and Gch1^{fl/fl}Tie2cre mothers were determined at birth. Weights were averaged per litter of animals (*P<0.05 n=51 to 75 pups from 7 to 10 litters per group).

FIG. 4 shows: Effect of endothelial cell BH4 deficiency on vascular function in pregnancy. Vascular function of isolated uterine arteries (UA) from non-pregnant (NP) and pregnant (P) mice at E18.5 day of gestation of both genotypes was determined using wire myography. (a) Diameters of uterine arteries, as determined by length-tension relationship at 100 mmHg, were significantly increased in pregnant UA from both genotypes. Vasoconstrictions in response to KCL response (45 mM) were significantly increased in pregnant UA from both genotypes (*P<0.05 n=6 to 8 animals per group); (b) Wall stress in uterine arteries from non-pregnant and pregnant wild-type and Gch1fl/flTie2cre mice. Wall stress (vasoconstriction to U46619 to media area; mN/mm²), was similar between the non-pregnant wild-type and non-pregnant Gch1^{fl/fl}Tie2cre mice. In pregnant uterine arteries, wall stress was significantly reduced in both wild-type and Gch1fl/flTie2cre mice, but with lesser extent in Gch1^{fl/fl}Tie2cre mice. (*P<0.05 n=6 to 8 animals per group); (c) Percentage contraction in response to U46619 in non-pregnant and pregnant mice from both genotypes (*P<0.05 n=6 to 8 animals per group); (d) Percentage contraction in response to U46619 in pregnant uterine arteries in the presence or absence of non-selective NOS inhibitor, L-NAME (*P<0.05 n=6 to 8 animals per group); (e) Endothelium-dependent vasodilatation to acetylcholine (Ach) in the presence or absence of L-NAME (*P<0.05 pregnant WT control vs pregnant WT+L-NAME; n=6 to 8 animals per group); (f) Endothelium-independent vasodilatation in response to the nitric oxide donor, sodium nitroprusside (SNP) (*P<0.05 n=6 to 8 animals per group); (g and h) EC50 and maximum contractions in response to U46619; (i and j) EC50 and maximum relaxations in response to Acetylcholine (Ach); (k and i) Contribution of NOS-derived vasodilators, prostacyclin, and endothelium-derived hyperpolarising factors (EDHF) in non-pregnant and pregnant from both genotypes. Endothelium-dependent vasodilatations to Ach were determined in the presence of cyclooxygenase inhibitor, indomethacin (10 μM) alone, or indomethacin and the nitric oxide synthase inhibitor, L-NAME (100 μM), or indomethacin, L-NAME and endothelium-derived hyperpolarising factor blockers (apamin and charybdotoxin) in non-pregnant wild-type and Gch1^{fl/fl} Tie2cre mice and pregnant wild-type and Gch1^{fl/fl}Tie2cre mice; (m) Percentage contribution of NOS-derived vasodilators, prostacyclin and EDHF (i.e. combined apamin-sensitive component (SKca) and Charybdotoxin-sensitive component (IKca and BKca)) in non-pregnant and pregnant of both genotypes (*P<0.05 n=5 to 6 animals per group).

FIG. 5 shows: Effect of endothelial cell BH4 deficiency on placental size and vascular remodelling in uterine arteries and spiral arteries in pregnancy. Vascular remodelling was analysed in embedded sections of uterine arteries (perfusion fixed at 100 mmHg) and placentas from non-pregnant and pregnant wild-type and Gch1^{fl/fl}Tie2cre mice. (a) Representative images of placental casts of the umbilical arterial and venous circulation from wild-type (left) and Gch1^{fl/fl}Tie2cre mice (right) at E18.5 day of gestation; (b) Representative micro-computed tomography (uCT) images (superior view) of placental casts of umbilical arterial and venous circulation from wild-type and Gch1^{fl/fl}Tie2cre mice (right) at E18.5 day of gestation; (c) Placentas from wild-type and Gch1^{fl/fl}Tie2cre mothers were collected and weighed at E18.5 day of gestation (*P<0.05 n=6 to 8 per group) (f) Representative images are shown α-smooth muscle actin (α-SMA) staining of uterine arteries. Opened arrow indicates internal elastic lamina and closed arrow indicates external elastic lamina. (g) Vascular remodeling and medial hypertrophy in uterine artery sections from non-pregnant (NP) and pregnant (P) wild-type and Gch1^{fl/fl}Tie2cre mice. Vascular remodelling was evaluated by quantification of lumen area, media area, vascular smooth muscle (VSM) area (by α-SMA immunostaining), VSM to lumen+media ratio, and media to lumen ratio. Quantification was performed by using Image Pro Plus software (*P<0.05 n=5 to 6 animals per group). (h) H&E and α-SMA staining of representative spiral arteries in the decidua of mouse placenta from wild-type and Gch1^{fl/fl}Tie2cre mice. (i) Quantification of lumen area and percentage medial smooth muscle cells of spiral arteries was quantified by α-SMA immunostaining and Image Pro Plus software. (* p<0.05 n=5 animals per group).

FIG. 6 shows: Supplementation of sepiapterin (a functional equivalent of BH4) and 5-MTHF rescues pregnant-induced hypertension and fetal growth restriction in pregnant mice with endothelial cell BH4 deficiency. Gch1^{fl/fl}Tie2cre and wild-type mice were treated with either oral BH4 (200 mg/kg/day, supplemented in chow), or oral BH4 (200 mg/kg/day) with 5-MTHF (15 mg/kg/day) or control diet for 3 days before timed-matings, and throughout the subsequent pregnancies. (a-c) HPLC analysis of BH4, BH2, and BH4/(BH2+B) (BH4/total biopterins ratio) in plasma from wild-type and Gch1^{fl/fl} Tie2cre mice treated with BH4 alone or BH4 with 5-MTHF or control at E18.5 day of gestation (*P<0.05 n=to 7 animals per group). (d-f) Systolic blood pressure in wild-type and Gch1^{fl/fl} Tie2cre mice treated with BH4 alone or BH4 with 5-MTHF or control diet before and during pregnancy was measured by noninvasive tail-cuff (*P<0.05n=5 to 7 animals per group). (g-i) fetal and placental weight determined at E18.5 of gestation (*P<0.05 n=32 to 43 fetuses from 5 to 7 litters per group). (j-I) Vascular function in uterine arteries from wild-type and Gch1^{fl/fl}Tie2cre mice treated with either BH4 alone or BH4+5-MTHF or control diet was assessed by wire myography. (j) Vasoconstriction in response to U46619 in wild-type and Gch1^{fl/fl} Tie2cre mice treated with or without BH4 and 5-MTHF. (k) Endothelium-dependent vasodilatation in response to Ach was rescued by supplementation of BH4 with 5-MTHF in Gch1^{fl/fl}Tie2cre mice. (I) Endothelium-independent vasodilatations in response to SNP were similar between the groups. P<0.05 (*) wild-type control vs. Gch1^{fl/fl}Tie2cre mice control, (#) Gch1^{fl/fl} Tie2cre control vs. Gch1^{fl/fl}Tie2cre mice treated with BH4+5-MTHF; n=4 to 6 animals per group).

FIG. 7 shows Plasma Biomarkers in Women with Normotensive (NT) or Pre-Eclamptic (PE) Pregnancies. (a and b) plasma anti-angiogenic markers soluble endoglin (sENG) and soluble fms-like tyrosine kinsase-1 (sFlt-1) were measured by enzyme-linked immunosorbent assay on 5 days after delivery (n=107 per group). Levels of sENG and sFlt-1 at 5 days post-partum are lower than on the final day of pregnancy, but are closely correlated and remain representative of pregnancy levels (see Yu et al. Hypertension 2016; 68:749-59). (c, d and e) Levels of BH4, total biopterins, and BH4/(BH2+B) (BH4/total biopterins ratio) were measured by HPLC in plasma from women with preeclampsia (n=38) and in normotensive controls (n=24) at baseline (3 months after pregnancy) and late pregnancy. Black symbols denote NT, blue symbols denote PE. * denotes p<0.05 for PE vs. NT, or late pregnancy vs. early pregnancy.

FIG. 8 shows: Endothelial cell-specific floxed allele excision in pregnant Tie2cre mouse uterine artery and placenta. Tie2cre mice were crossed with floxed TdTomato reporter mice. Female Tie2cre/TdTomato mice underwent timed matings with WT male mice. Uterine arteries and placental tissues were harvested at E18.5 day of gestation for fluorescence microscopy. Red Tdt fluorescence highlights endothelial cells in (a) uterine arteries and (b) decidual spiral arteries (*), respectively. Nuclei are stained blue with DAPI.

FIG. 9 shows: Liver and Urine Biopterin Levels in Wild Type and Gch1^{fl/fl}Tie2cre Mice in Pregnancy. (a-e) Levels of BH4, BH2, B (total biopterins) were measured by HPLC in liver tissue homogenates obtained from wild type (WT) and Gch1^{fl/fl}Tie2cre mice, both non-pregnant and at the end of pregnancy. Total biopterins (BH4+BH2+B) and BH4/(BH2+B) ratio were calculated (n=6 animals per group). (f-g) Concentration of biopterins were measured by HPLC in urine samples from wild type (WT) and Gch1^{fl/fl}Tie2cre mice, both non-pregnant and at the end of pregnancy. Total biopterins (BH4+BH2+B) and BH4/(BH2+B) ratio were calculated (n=6 animals per group). (*) denotes p<0.05 for WT vs. Gch1^{fl/fl}Tie2cre, or pregnant vs. non-pregnant (n=6 animals per group).

FIG. 10 shows: Urinary Protein and Plasma Protein and Liver enzyme Levels in Wild Type and Gch1_{fl/fl}Tie2cre Mice in Pregnancy. (a-c) Levels of creatinine and total protein were measured by clinical chemistry analyser in urine obtained from wild type (WT) and Gch1^{fl/fl}Tie2cre mice, both non-pregnant and at the end of pregnancy. Urinary protein/creatinine ratio were calculated (n=6 animals per group). (d-g) Levels of alanine transaminase (ALT), Aspartate transaminase (AST), creatinine and albumin were measured by clinical chemistry analyser in plasma obtained from wild type (WT) and Gch1^{fl/fl}Tie2cre mice, both non-pregnant and at the end of pregnancy (n=6 animals per group).

FIG. 11 shows: Urine Biopterin/creatinine ratio and plasma biopterin/urine biopterin ratio in Wild Type and Gch1^{fl/fl}Tie2cre Mice in Pregnancy. (a-e) Urine BH4/creatinine ratio, BH2/creatinine ratio, B/creatinine ratio, total biopterin/creatine ratio obtained from wild type (WT) and Gch1^{fl/fl}Tie2cre mice, both non-pregnant and at the end of pregnancy (n=6 animals per group). * denotes p<0.05 for WT vs. Gch1^{fl/fl}Tie2cre, or pregnant vs. non-pregnant. (f-g) Plasma BH4/urine BH4 ratio and plasma total biopterins to urine total biopterins ratio obtained from wild type (WT) and Gch1^{fl/fl}Tie2cre mice, both non-pregnant and at the end of pregnancy (n=6 animals per group). (*) denotes p<0.05 for WT vs. Gch1^{fl/fl}Tie2cre, or pregnant vs. non-pregnant.

FIG. 12 shows: Renal Histology in Gch1^{fl/fl}Tie2cre mice. Kidneys were harvested from pregnant wild type (WT) and Gch1^{fl/fl} Tie2cre mice, fixed and processed for histology. Sections were stained with periodic acid-Schiff (PAS), hematoxylin and eosin (H&E) or Masson trichrome stains. Dimensions and areas were measured using Image J. (a) Macroscopic images of kidney saggital cross sections from WT and Gch1^{fl/fl}Tie2cre. (b-f) Histologic measurements of kidney length, width, total area and cortical and medullary area. (g) Histologic images (×40 magnification, bar=30 um) of renal glomeruli from WT and Gch1^{fl/fl} Tie2cre mice. (h and i) Quantification of glomerular area and Bowman's space area from multiple glomeruli (n=6 to 7 animals per group).

FIG. 13 shows: Cardiovascular and behavioural activities in freely moving Wild Type and Gch1^{fl/fl}Tie2cre mice during last day of gestation. Twenty-four-hour telemetric recording of blood pressure and heart rate were performed in the conscious mice with telemetry system. Average mean arterial blood pressure and heart rate in 1 min time interval were plotted continuously for 24 hr from 0000 hour to 1200 hour of the next day. (a-e) Shown are 24 hour recording of mean arterial pressure, systolic pressure, diastolic pressure, heart rate, and activity count between E17.5-18.5 day of gestation. Shaded areas represent dark period when the light was switched off (2000 hour to 0800 hours of the next day).

FIG. 14 shows; Telemetry Blood Pressure in Pregnancy in Wild Type and Gch1^{fl/fl}Tie2cre mice. Female mice, either wild type (WT) or Gch1^{fl/fl}Tie2cre mice or WT male mice (to generate genetically matched litters), and blood pressure was measured during pregnancy by blood pressure telemetry. (a-c) Mean arterial pressure, systolic pressure, and diastolic pressure were measured during pregnancy. Both systolic and mean blood pressure at E18.5 days of gestation in Gch1^{fl/fl}Tie2cre mice, were mated with Gch1^{fl/fl}Tie2cre mice female mice were significantly higher than those in the wild-type littermate controls. (n=5 and n=7, respectively) (t) Denotes p<0.05 vs. WT; (*) denotes p<0.05 vs. baseline blood pressure. (d-e) Heart rate and activity throughout pregnancy for Gch1^{fl/fl} Tie2cre or WT female mice (n=5 and n=7, respectively).

FIG. 15 shows: Blood Pressure Changes in Pregnancy in Wild Type and Gch1^{fl/fl}Tie2cre mice Matched for Baseline Blood Pressure. Female mice, either wild type (WT) or Gch1^{fl/fl}Tie2cre, were mated with Gch1^{fl/fl}Tie2cre or WT male mice (to generate genetically matched litters), and blood pressure was measured every 3 days during pregnancy by tail cuff plethysmography. (a) Mean change in blood pressure between early pregnancy (e2.5) and late pregnancy (e18.5) for WT or Gch1^{fl/fl}Tie2cre female mice (n=5 and n=6, respectively) (b) Blood pressure profiles throughout pregnancy for WT or Gch1^{fl/fl}Tie2cre female mice (n=5 and n=6, respectively) that were selected from the cohort to ensure equal blood pressure at baseline. Even after this baseline covariate adjustment, the increase in BP during pregnancy remained much greater in the Gch1^{fl/fl}Tie2cre mice compared with WT mice. (*) Denotes p<0.05 vs. WT; t denotes p<0.05 vs. baseline blood pressure.

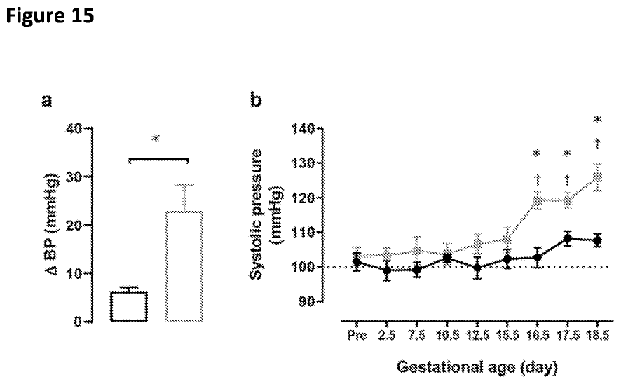

FIG. 16 shows: Biopterin Levels in Mice with Heterozygous Deletion of Gch1 in Endothelial Cells (i.e. Gch1^{fl/+}Tie2cre Mice) in Pregnancy. Mice with heterozygous deletion of Gch1 in endothelial cells (i.e. Gch1^{fl/+}Tie2cre mice) were generated by crossing Gch1^{fl/fl}Tie2cre mice with WT (i.e. Gch1+/*) mice. Female Gch1^{fl/+}Tie2cre mice were mated with WT male mice. (a) Genomic PCR shows the presence of the Gch1floxed and WT alleles in both Gch1^{fl/+} (WT) and Gch1^{fl/+}Tie2cre mice. (b-u) Levels of BH4, BH2, B (total biopterins) were measured by HPLC in tissue homogenates obtained from wild type (WT) and Gch1^{fl/+}Tie2cre, at the end of pregnancy. Total biopterins (BH4+BH2+B) and BH4/(BH2+B) ratio were calculated. Shown are biopterin levels in aorta (b-f), lung (g-k), liver (l-p) and pregnant uterine artery (q-u). (*) Denotes p<0.05 vs. WT.

FIG. 17 shows: Blood Pressure and Fetal Growth in Pregnant Mice with Heterozygous Deletion of Gch1 in Endothelial Cells (i.e. Gch1^{fl/fl}Tie2cre Mice). (a and b) Blood pressure and heart rate at baseline (pre-pregnancy) (n=6 to 8 animals per group). (c and d) Blood pressure and heart rate before and during pregnancy (n=6 to 8 animals per group). (e) Litter size (number of embryos) was not different between wild-type and Gch1^{fl/+}Tie2cre mice (n=6 to 8 animals per group). (f-h) Fetuses at gestation day 18.5 were significantly smaller from pregnant Gch1^{fl/+}Tie2cre compared to wild-type females. (*) Denotes p<0.05 vs. WT; n=6 to 8 animals per group.

FIG. 18 shows: Organ Weights from wild type and Gch1^{fl/fl}Tie2cre mice in Pregnancy: Organ weights: (a) Hearts, (b) lungs, (c) liver, (d) kidney and (e) spleens from non-pregnant mice and pregnant (E18.5 day gestation) wild-type and Gch1_{fl/fl}Tie2cre mice. (*) Denotes p<0.05; n=6 animals per group.

FIG. 19 shows: Breeding Strategy to Generate Matched Litters for Studies of Maternal Gch1 Deletion in Pregnancy: a) Schematic of breeding pairs of either female WT (i.e. Gch1_{fl/fl}) crossed with male Gch1^{fl/fl}Tie2cre mice, or female Gch1^{fl/fl}Tie2cre crossed with male WT mice. Both breeding pairs produce the same littermates composed of WT or Gch1_{fl/fl}Tie2cre offspring in 1:1 ratio, with the key difference being that the genetically matched offspring are either born to a WT mother or a Gch1^{fl/fl}Tie2cre mother. (b) Systolic blood pressure, measured by non-invasive tail-cuff, in wild-type females mated with either wild-type males or Gch1_{fl/fl}Tie2cre males before and during pregnancy. (n=7 to 10 animals per group). (c and d) fetal and placental weights from Gch1^{fl/fl}Tie2cre mothers were determined according to fetus genotypes. No difference in fetal and placental weights between wild-type fetus and Gch1^{fl/fl}Tie2cre fetus from Gch1^{fl/fl}Tie2cre mothers. (e) No difference in litter size between wild-type and Gch1^{fl/fl}Tie2cre mothers at birth. (f) Live pups from wild-type and Gch1^{fl/fl}Tie2cre mothers were collected and measured at birth.

FIG. 20 shows: Fetal and placental weights of male and female fetuses from Wild Type and Gch1^{fl/fl}Tie2cre Mothers. (a) Representative of sex of the embryos from wild-type and Gch1^{fl/fl} Tie2cre mothers was genotyped using Zfy primer to detect Y chromosome. (b and c) Fetal and placental weights from wild-type and Gch1^{fl/fl}Tie2cre mothers were collected at E18.5 day of gestation and weighed according to fetus genotypes and genders. Male and female fetuses from pregnant Gch1^{fl/fl}Tie2cre mothers were significantly smaller compared to wildtype mothers. No male-female difference in the reduction in fetal weight. (*) denotes P<0.05; 25 males and 22 females from n=4 WT mothers and n=4 Gch1^{fl/fl}Tie2cre mothers.

FIG. 21 shows: Passive wall tension curves in uterine arteries from wild-type and Gch1_{fl/fl}Tie2cre mice at E18.5 day of gestation. Uterine arteries from pregnant wild-type and Gch1^{fl/fl} Tie2cre mice were dissected and 2 mm segments were mounted on a wire myograph in calcium-free KHB buffer. Passive tension was significantly increased in uterine arteries from Gch1^{fl/fl}Tie2cre mice compared to wild-type controls, indicating an inward remodelling or altered compliance in these vessels. (*) Denotes p<0.05 vs. WT; n=5 to 6 animals per group.

FIG. 22 shows: Vasomotor function in aortas from non-pregnant and pregnant wild-type and Gch1^{fl/fl}Tie2cre mice at E18.5 day of gestation. Female mice, either wild type (WT) or Gch1^{fl/fl}Tie2cre, were mated with Gch1^{fl/fl}Tie2cre or WT male mice to generate genetically matched litters. Vascular function of isolated uterine arteries (UA) from non-pregnant (NP) and pregnant (P) mice at E18.5 of both genotypes was determined using a wire myography. (a) Absolute contraction in response to KCL response (mN). (b) Percentage contraction in response to phenylephrine in non-pregnant and pregnant mice from both genotypes. (c and d) Endothelium-dependent vasodilatation to acetylcholine (Ach) in the presence or absence of L-NAME. (e) Endothelium-independent vasodilatation in response to the nitric oxide donor, sodium nitroprusside (SNP). (f and g) EC₅₀ and Maximum contraction in response to phenylephrine (PE). (h and i) EC₅₀ and maximum relaxation in response to acetylcholine (Ach). (*) Denotes p<0.05 pregnant Gch1^{fl/fl}Tie2cre vs. pregnant WT; (#) denotes p<0.05 non-pregnant mice vs. pregnant mice (n=6 animals per group).

FIG. 23 shows: Contribution of prostacyclin, eNOS-derived vasodilators, and EDHF in pregnant wild-type and Gch1^{fl/fl}Tie2cre uterine arteries at E18.5 day of gestation. Isometric tension studies of uterine arteries from pregnant WT (a) and Gch1^{fl/fl}Tie2cre mice (b) were examined using a wire myograph. Endothelium-dependent vasodilatations to acetylcholine (ACh) were determined in the presence of the cyclooxygenase inhibitor, indomethacin (10 μM) alone, or indomethacin and the nitric oxide synthase inhibitor, L-NAME (100 μM), or indomethacin, L-NAME and EDHF blockers (apamin and charybdotoxin). (c) Percentage contribution of apamin-sensitive component (SKca), charybdotoxin-sensitive component (IKca and BKca) and combination of apamin and charybdotoxin sensitive component (i.e. EDHF). (*) denotes P<0.05, n=5 animals per group.

FIG. 24 shows: BH4 measurements in uterine arteries isolated from pregnant wild-type and Gch1^{fl/fl}Tie2cre treated with sepiapterin (a functional equivalent of BH4) and 5-MTHF during pregnancy. Pregnant Gch1^{fl/fl}Tie2cre and wild-type mice were treated with either oral BH4 (200 mg/kg/day) supplementation, or oral BH4 (200 mg/kg/day) with 5-MTHF (15 mg/kg/day) or control diet for 3 days before timed-matings, and throughout the subsequent pregnancies. Uterine arteries were harvested at E18.5 days of gestation. (a-e) Levels of BH4, BH2, B (total biopterins) were measured by HPLC in uterine arteries obtained from wild-type and Gch1^{fl/fl}Tie2cre mice treated with BH4 alone or BH4 with 5-MTHF or control at E18.5 day of gestation (*) P<0.05, n=5 to 7 animals per group.

FIG. 25 shows: Placental GTPCH and BH4 measurements in wild-type and Gch1^{fl/fl}Tie2cre mice at E18.5 day of gestation. (a) Representative immunoblots showing GTPCH protein in in placentas from wild-type (WT) and Gch1^{fl/fl}Tie2cre mice. Western blot controls include sEND cells (mouse endothelial cell line). (b) Placental BH4 and total biopterins, measured by HPLC, were significantly reduced in placentas from Gch1^{fl/fl}Tie2cre mice were compared with WT littermates (*) denotes P<0.05, n=5-7 animals per group; for each animal 2-3 placentas with unknown fetal genotype were selected for BH4 measurements.

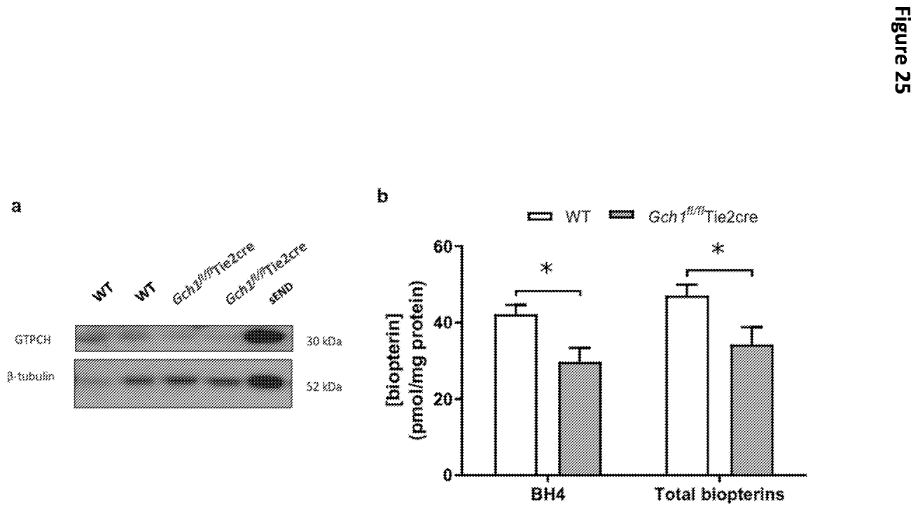

FIG. 26 shows: Supplementation of 5-MTHF but not folic acid rescues pregnant-induced hypertension in pregnant mice with endothelial cell BH4 deficiency. Gch1^{fl/fl}Tie2cre (squares) and wild-type (circles) mice were treated with either oral folic acid (15 mg/kg/day, supplemented in chow) or oral 5-MTHF (15 mg/kg/day) or control diet for 3 days before timed-matings and throughout the subsequent pregnancies. (a-c) Systolic blood pressure in wild-type and Gch1^{fl/fl} Tie2cre mice treated with folic acid alone (n=3 per animals per group) or 5-MTHF (n=5-7 animals per group) or control diet (n=5-animals per group) before and during pregnancy was measured by non-invasive tail-cuff. (*) denotes P<0.05 wild-type control vs Gch1^{fl/fl}Tie2cre mice control; (#) denotes P<0.05 non-pregnant Gch1^{fl/fl}Tie2cre mice vs pregnant Gch1^{fl/fl}Tie2cre mice.

FIG. 27 shows the effect of 5MTHF supplementation on BH4 levels in mouse endothelial cells (sEnd.1). sEnd.1 cells were exposed to either MTX (1 μm) alone or 5MTHF (10 μm) alone or MTX+5MTHF for 16 h at 37° C., and intracellular biopterin levels were quantified by HPLC. (*, p<0.05 comparing control vs MTX, #, p<0.05 comparing MTX vs MTX+5MTHF, n=6 for all experiments).

FIG. 28 shows the effect of folic acid supplementation in mouse endothelial cells. sEnd.1 cells were exposed to either MTX (1 μm) alone or Folic acid (10 μm) alone or MTX+ Folic acid for 16 h at 37° C., and intracellular biopterin levels were quantified by HPLC. (*, p<0.05 comparing control vs MTX, n=3 for all experiments).

FIG. 29 shows the effect of 5MTHF supplementation on GTPCH and eNOS protein expression in mouse endothelial cell line. sEnd.1 cells were exposed to either MTX (1 μm) alone or 5MTHF (10 μm) alone or MTX+5MTHF for 16 h at 37° C., and intracellular biopterin levels were quantified by HPLC. (A) Representative immunoblots with corresponding quantitative data (B) showing GTPCH protein in endothelial cells treated either MTX alone, 5MTHF alone or 5MTHF+MTX. Corresponding immunoblots for beta-tubulin (loading control) (*, p<0.05, n=6 per group).

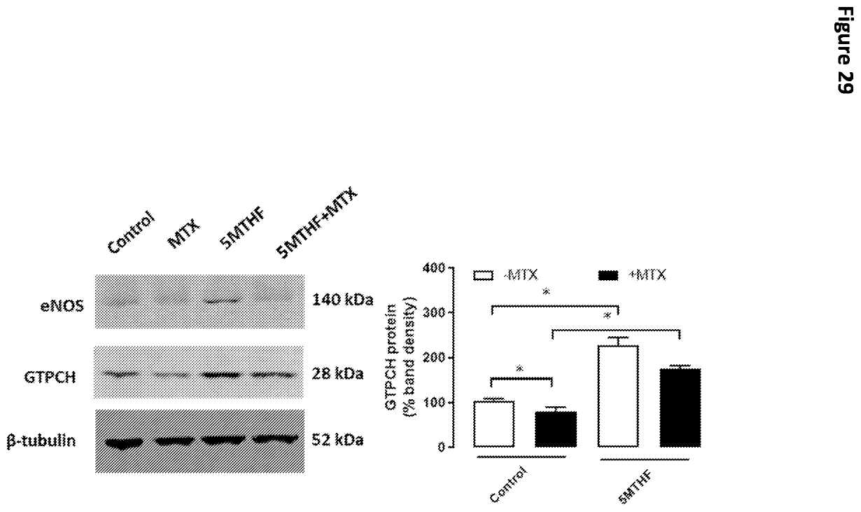

FIG. 30 shows the effect of Ascorbic acid supplementation in mouse endothelial cells. sEnd.1 cells were exposed to either MTX (1 μm) alone or ascorbic acid (10 μm) alone or MTX+5MTHF for 16 h at 37° C., and intracellular biopterin levels were quantified by HPLC. (*, p<0.05, n=3-6 per group).

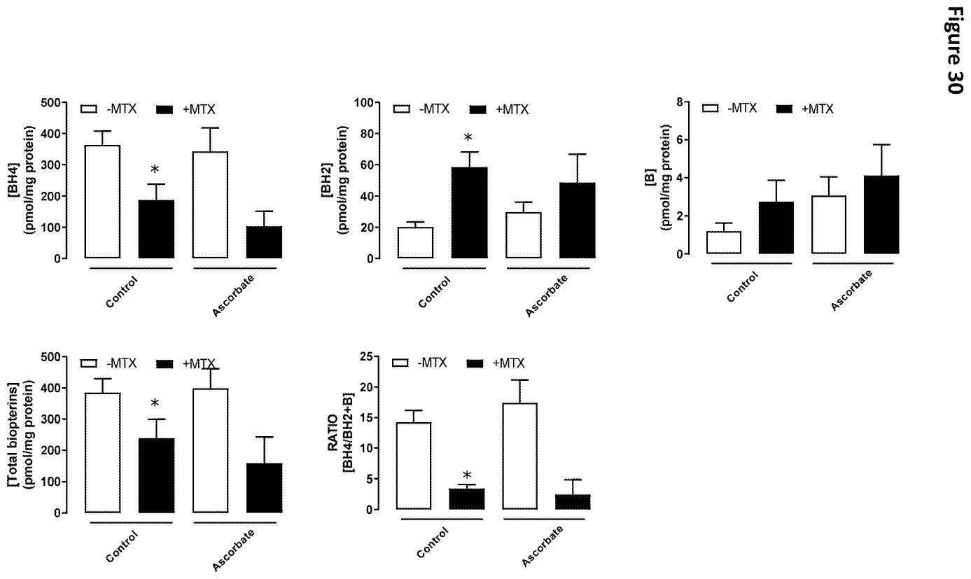

FIG. 31 shows DHF, THF, and 5MTHF measurement using HPLC. sEnd.1 cells were exposed to either MTX (1 μm) alone or 5MTHF (10 μm) alone or MTX+5MTHF for 16 h at 37° C., and intracellular DHF, THF and 5MTHF levels were quantified by HPLC. (*, p<0.05 comparing control vs MTX, #, p<0.05 comparing MTX vs MTX+5MTHF, n=6 for all experiments).

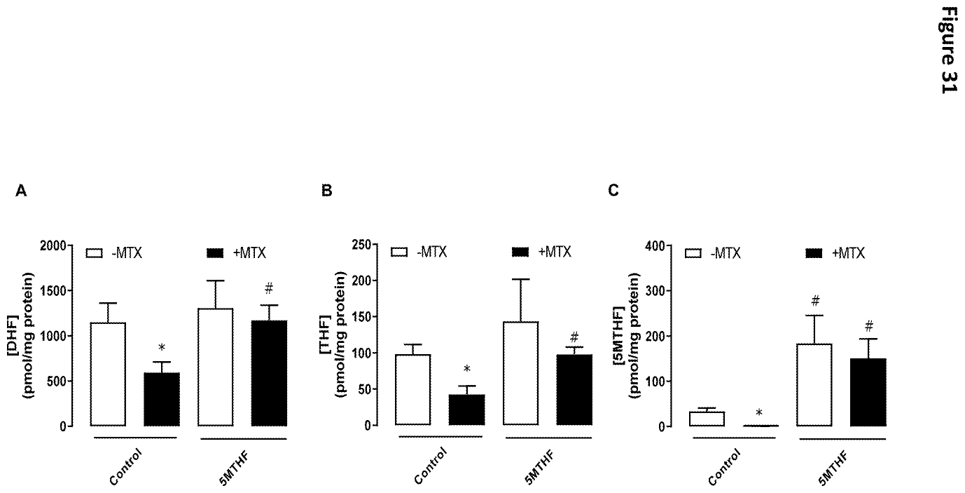

FIG. 32 shows the Effect of 5MTHF Supplementation on ROS productions in Mouse Endothelial cells. Superoxide and other reactive oxygen species (ROS) productions detected by dihydroethidine (DHE) high-performance liquid chromatography (HPLC). Superoxide and other ROS productions as measured by 2-hydroxyethidium (2-HE) and ethidium, respectively. sEnd.1 cells were exposed to either MTX (1 μm) alone or 5MTHF (10 μm) alone or MTX+5MTHF for 16 h at 37° C., and 2-HE and ethidium levels were quantified by HPLC. (*, p<0.05, n=6 for all experiments).

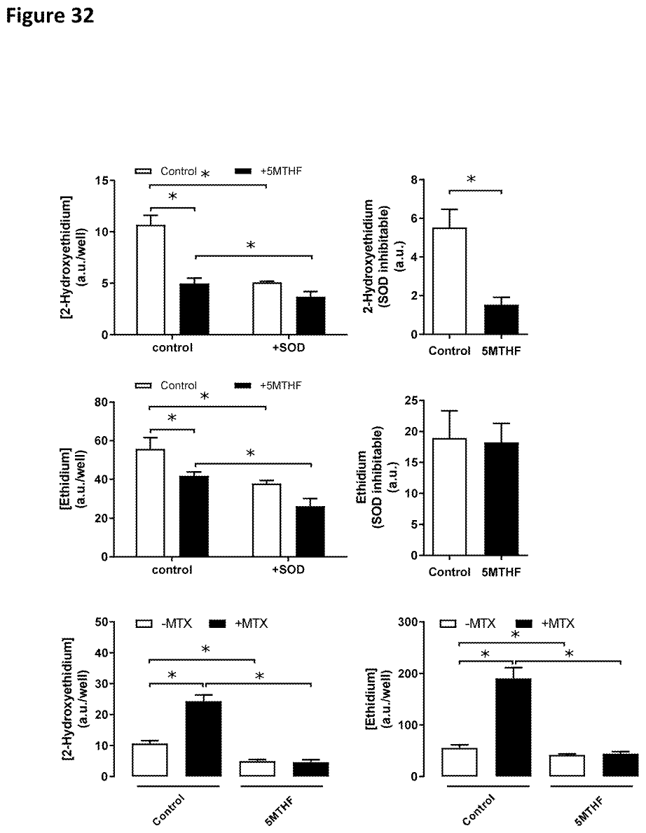

FIG. 33 shows the Effect of Folic Acid Supplementation on ROS productions in Mouse Endothelial cells. Superoxide and other reactive oxygen species (ROS) productions detected by dihydroethidine (DHE) high-performance liquid chromatography (HPLC). Superoxide and other ROS productions as measured by 2-hydroxyethidium (2-HE) and ethidium, respectively. sEnd.1 cells were exposed to either MTX (1 μm) alone or folic acid (10 μm) alone or MTX+5MTHF for 16 h at 37° C., and 2-HE and ethidium levels were quantified by HPLC. (*, p<0.05, n=3 for all experiments).

FIG. 34 shows the Effect of 5MTHF Supplementation on NOS Activity in Mouse Endothelial cells. Conversion of 14C arginine to 14C citrulline was used as a measure of endothelial nitric oxide synthase activity (eNOS). sEnd.1 cells were exposed to either methotrexate (MTX); 1 μm) alone or 5MTHF (10 μm) alone or MTX+5MTHF for 16 h at 37° C., and conversion of 14C arginine to 14C citrulline was quantified by HPLC. (*, p<0.05, n=6 for all experiments).

FIG. 35 shows the effect of Sepiapterin (a functional precursor of BH4) supplementation in Human Endothelial cells. HUVECS were exposed to either methotrexate (MTX; 1 μm) alone or sepiapterin (1 μm) alone or MTX+sepiapterin for 16 h at 37° C., and intracellular biopterin levels were quantified by HPLC. (*, p<0.05 comparing control vs MTX, #, p<0.05 comparing MTX vs MTX+5MTHF, n=6 for all experiments).

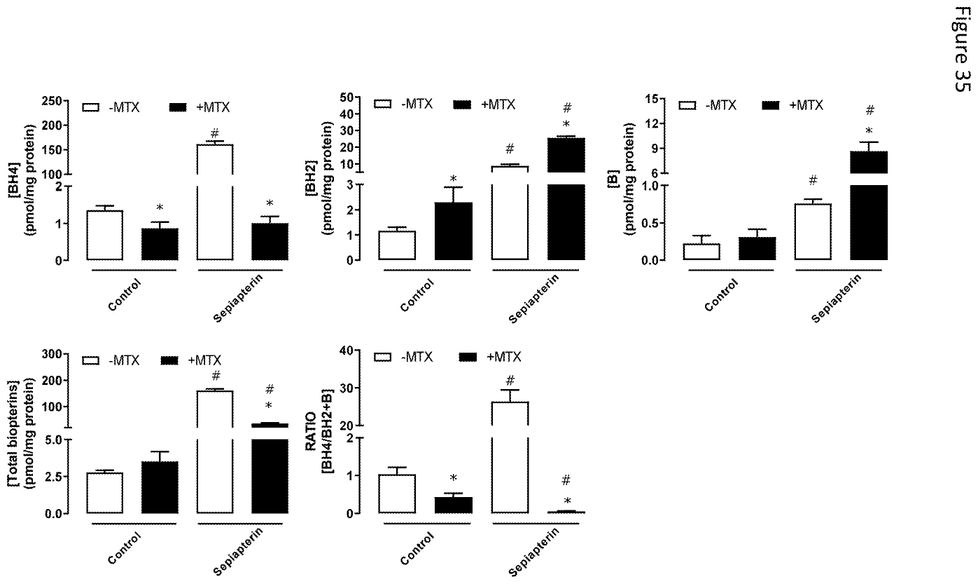

FIG. 36 shows Arcofolin (5-MTHF) supplementation increases BH4 levels in Human Endothelial cells. HUVECS were exposed to either MTX (1 μm) alone or 5MTHF (10 μm) alone or MTX+5MTHF for 16 h at 37° C., and intracellular BH4 and oxidised biopterins (BH2 and biopterin) were determined by high-performance liquid chromatography (HPLC). (*, p<0.05 comparing control vs MTX, #, p<0.05 comparing MTX vs MTX+5MTHF, n=6 for all experiments).

FIG. 37 shows the Effect of 5MTHF Supplementation on ROS productions in Human Endothelial cells. Superoxide and other reactive oxygen species (ROS) productions detected by dihydroethidine (DHE) high-performance liquid chromatography (HPLC). Superoxide and other ROS productions as measured by 2-hydroxyethidium (2-HE) and ethidium, respectively. HUVECS were exposed to either MTX (1 μm) alone or 5MTHF (10 μm) alone or MTX+5MTHF for 16 h at 37° C., and 2-HE and ethidium levels were quantified by HPLC. (*, p<0.05, n=6 for all experiments).

FIG. 38 shows further data on the Effect of 5MTHF Supplementation on ROS productions in Human Endothelial cells. Superoxide and other reactive oxygen species (ROS) productions detected by dihydroethidine (DHE) high-performance liquid chromatography (HPLC). Superoxide and other ROS productions as measured by 2-hydroxyethidium (2-HE) and ethidium, respectively. HUVECS were exposed to either MTX (1 μm) alone or 5MTHF (10 μm) alone or MTX+5MTHF for 16 h at 37° C., and 2-HE and ethidium levels were quantified by HPLC. (*, p<0.05, n=6 for all experiments).

FIG. 39 shows effects of supplementation of 5MTHF at 10.5 day of gestation (Mid-pregnancy) on pregnant-induced hypertension in pregnant mice with endothelial cell BH4 deficiency. Gch1fl/flTie2cre and wild-type mice were treated with either oral 5MTHF (15 mg/kg/day or control diet at 10.5 day of gestation. Systolic blood pressure was measured by non-invasive tail-cuff in wild-type (WT) and Gch1fl/flTie2cre mice before and during pregnancy. Graphs show mean blood pressures from n=3 to 6 animals per group, measured throughout pregnancy at the time points shown (* P<0.05 comparing genotype; #P<0.05 comparing baseline blood pressure).

FIG. 40 shows supplementation of 5MTHF at 10.5 day of gestation (Mid-pregnancy) rescues pregnant-induced hypertension and fetal growth restriction in pregnant mice with endothelial cell BH4 deficiency. (A) Systolic blood pressure was measured by non-invasive tail-cuff in wild-type (WT) and Gch1fl/flTie2cre mice before and during pregnancy, with each data point showing the mean blood pressure from each animal at time points basal (pre-pregnancy) or E18.5 (day 18.5 of pregnancy). (* P<0.05 comparing genotype; #P<0.05 comparing baseline blood pressure; n=3 to 6 animals per group) (B) shows litter size (number of pups born), fetal weights and placental weights in wild-type (WT) and Gch1fl/flTie2cre mice following treatment with either 5-MTHF or control diet.

FIG. 41 shows supplementation of 5MTHF at 16.5 day of gestation (late-pregnancy) rescues pregnant-induced hypertension in pregnant mice with endothelial cell BH4 deficiency. Gch1fl/flTie2cre and wild-type mice were treated with either oral 5MTHF (15 mg/kg/day) or control diet at 16.5 day of gestation. Systolic blood pressure was measured by non-invasive tail-cuff in wild-type (WT) and Gch1fl/flTie2cre mice before and during pregnancy. (* P<0.05 comparing genotype; #P<0.05 comparing baseline blood pressure; n=6 to 7 animals per group).

### DESCRIPTION

The invention will now be described with reference to specific common features of the aspects and embodiments defined herein above. Any feature in any section may be combined with any other feature in any other section, and with any aspect or embodiment in any workable combination.

It is to be noted that the term “a” or “an” entity refers to one or more of that entity. As such, the terms “a” (or “an”), “one or more,” and “at least one” can be used interchangeably herein.

As used herein, the terms “treat” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired, except where the subject is defined as a ‘healthy subject’. Mammalian subjects include humans; domestic animals; farm animals; such as dogs, cats, guinea pigs, rabbits, rats, mice, horses, cattle, cows, and so on. The term ‘subject’ is defined further hereinbelow.

### Reduced Folate

The present invention is based on the discovery that a reduced folate alone, or optionally in combination with BH4, a precursor or functional equivalent thereof, can treat disorders associated with low BH4 bioavailability.

The term “folate” as used herein refers to compounds based on a pteroate group, which is coupled through a peptide bond to a glutamic acid. Preferred representatives of folates as used herein are based on a folate skeleton, i.e. pteroyl-glutamic acid resp. N-[4-[[(2-amino-1,4-dihydro-4-oxo-6-pteridinyl)methyl]amino]benzoyl]-L-glutamic acid, and derivatives thereof. Suitably the reduced folate may be selected from: dihydrofolic acid (DHF); 5-formyltetrahydrofolic acid (5-FTHF); tetrahydrofolic acid (THF); 5,10-methylenetetrahydrofolic acid (5,10-CH₂-THF); 5,10-methenyltetrahydrofolic acid (5,10-CH-THF); 10-formyltetrahydrofolic acid (10-FTHF), or 5-methyltetrahydrofolic acid (5-MTHF) or a pharmaceutically acceptable salt thereof or a polyglutamate thereof. In a preferred embodiment the folate is in its natural stereoisomeric form, such as e.g. 5-methyl-(6S)-tetrahydrofolic acid, 5,10-methenyl-(6R)-tetrahydrofolic acid or 5-formyl-(6S)-tetrahydrofolic acid.

Suitably the reduced folate may be selected from: 5,10-CH-THF; 5-FTHF; 10-FTHF, and 5-MTHF or a pharmaceutically acceptable salt thereof.

In one embodiment, the reduced folate is 5-MTHF or a pharmaceutically acceptable salt thereof. In one embodiment, the reduced folate is 5-MTHF.

In one embodiment, the reduced folate is in its natural stereoisomeric form. In one embodiment the reduced folate is 5-methyl-(6S)-tetrahydrofolic acid.

5-MTHF as used herein refers to 5-methyltetrahydrofolate, otherwise known as: Levomefolic acid, L-5-MTHF, L-methylfolate, L-5-methyltetrahydrofolate, (6S)-5-methyltetrahydrofolate, or (6S)-5-MTHF.

Suitably the reduced folate may be a pharmaceutically acceptable salt of a reduced folate. Suitable pharmaceutically acceptable salts will be known to those skilled in the art of pharmacy. Suitable pharmaceutically acceptable salts may include a calcium, magnesium, sodium, potassium, or ammonium salt. A suitable calcium salt of 5-MTHF is Metafolin®. A suitable sodium salt is Arcofolin®.

### BH4, a Precursor, or a Functional Equivalent Thereof

The present invention is based on the discovery that a reduced folate can be used to protect BH4 from oxidation to BH2. Providing a reduced folate alone, or in combination with BH4, a precursor, or a functional equivalent thereof, can raise in vivo BH4 levels and treat disorders associated with low BH4 bioavailability.

‘BH4’ as used herein refers to tetrahydrobiopterin, otherwise known as sapropterin.

Suitably BH4, or a precursor or functional equivalent thereof, may be provided as a pharmaceutically acceptable salt. Suitable pharmaceutically acceptable salts will be known to those skilled in the art of pharmacy. Suitable pharmaceutically acceptable salts in relation to BH4 may include a salt with a nonorganic or organic acid of BH4. Suitable BH4 salts include BH4 salts of acetic acid, citric acid, oxalic acid, tartaric acid, fumaric acid, and mandelic acid. A suitable chloride salt of BH4 is sapropterin dihydrochloride (BH4*2HCL), otherwise known as Kuvan® or Biopten®.

‘Precursor’ as used herein refers to any compound which may be converted into the specified chemical by one or more metabolic reactions. Suitably a BH4 precursor may include any compound which may be converted to BH4 by one or more metabolic reactions, suitably one or more enzymatic reactions. Suitably a BH4 precursor may include any compound which is within the pterin metabolic pathway. Suitably a BH4 precursor may include any compound which is within the BH4 biosynthetic pathway. Suitably a BH4 precursor may include, for example: GTP; NH2TP; PTP; oxo-PH4; 7,8-BH2; HO-BH4; q-BH2, L-arginine and/or L-citrulline.

‘Functional equivalent’ as used herein refers to any compound which is capable of, or which does perform, the same function as the specified chemical performs in vivo. Suitably a BH4 functional equivalent may include any compound which is capable of functioning as a cofactor for hydroxylase enzymes or synthase enzymes. Suitably as a cofactor for nitric oxide synthase or suitably as a cofactor for aromatic amino acid hydroxylase enzymes, suitably as a cofactor for biopterin-dependent aromatic amino acid hydroxylases such as, for example: phenylalanine 4-hydroxylase, tyrosine 3-hydroxylase, and tryptophan 5-hydroxylase. Suitably a functional equivalent of BH4 may include, for example: neopterin; sepiapterin; biopterin; and primapterin.

Suitably functional equivalents may also be precursors of BH4.

In one embodiment, the BH4, a precursor, or a functional equivalent thereof, comprises sepiapterin. In one embodiment, the BH4, a precursor, or a functional equivalent thereof, is provided as sepiapterin.

Disorders Associated with Low BH4 Bioavailability

The present invention is based on prevention or treatment of disorders associated with low BH4 bioavailability.

By low bioavailability it is meant low levels of BH4 when compared to a healthy subject. Suitably the low levels of BH4 may be in any tissue or cell type when compared to a healthy subject. Suitably the low levels of BH4 may be in endothelial cells when compared to a healthy subject. Suitably therefore, low BH4 bioavailability refers to a reduction in BH4 bioavailability, suitably a low level of BH4.

In one embodiment, low BH4 bioavailability comprises systemic low BH4 bioavailability, or local low BH4 bioavailability. Suitably local low BH4 bioavailability may comprise a reduction in BH4 at the tissue level or cellular level, for example low tissue BH4 bioavailability or low cellular BH4 bioavailability in specific tissues or cells. In one embodiment, low BH4 bioavailability may comprise low endothelial BH4 bioavailability. Suitably low BH4 bioavailability may result from a BH4 deficiency. Suitably low BH4 bioavailability may also result from a genetic BH4 deficiency. Alternatively or additionally, low BH4 bioavailability may result from biochemical factors. For example impaired BH4 transport or BH4 uptake into cells, or increased oxidation or reduction of BH4, for example.

In one embodiment, the disorder may be BH4 deficiency (tetrahydrobiopterin deficiency) itself, or a disorder which is associated with a BH4 deficiency. Suitably any references herein to ‘low BH4 bioavailability’ may equally refer to ‘a BH4 deficiency’. Suitably either a deficiency as such, or low bioavailability, may result from similar or the same causes, or combination of causes.

Suitably, low BH4 bioavailability may result from impaired BH4 synthesis. Suitably impaired BH4 synthesis may be caused by impaired enzyme activity, suitably within the pterin biosynthetic pathway, suitably within the BH4 biosynthetic pathway. Suitably impaired BH4 synthesis may be caused by one or more impaired enzymes within the pterin biosynthetic pathway. Suitably impaired BH4 synthesis may also be caused by one or more impaired enzymes within the BH4 biosynthetic pathway. BH4 deficiency can also be caused by a deficiency of the enzyme dihydrobiopterin reductase (DHPR), whose activity is needed to replenish quinonoid-dihydrobiopterin back into its tetrahydrobiopterin form.

Suitably therefore, disorders associated with low BH4 bioavailability may be substantially the same as disorders associated with impaired BH4 synthesis. Suitably therefore, disorders associated with low BH4 bioavailability may be substantially the same as disorders associated with one or more impaired enzymes within the pterin biosynthetic pathway. Suitably therefore, disorders associated with low BH4 bioavailability may also be substantially the same as disorders associated with one or more impaired enzymes within the BH4 biosynthetic pathway.

Suitably the present invention is for the prevention or treatment of a disorders associated with low BH4 bioavailability caused by impaired activity of one or more enzymes within the pterin biosynthetic pathway. Suitably the present invention is also for the prevention or treatment of a disorders associated with low BH4 bioavailability caused by impaired activity of one or more enzymes within the BH4 biosynthetic pathway. Suitably the present invention is for the prevention or treatment of low BH4 bioavailability caused by impaired activity of one or more enzymes within the pterin biosynthetic pathway. Suitably the present invention is also for the prevention or treatment of low BH4 bioavailability caused by impaired activity of one or more enzymes within the BH4 biosynthetic pathway. Suitably the present invention is for the prevention or treatment of a disorder caused by impaired activity of one or more enzymes within the pterin biosynthetic pathway. Suitably the present invention is for the prevention or treatment of a disorder caused by impaired activity of one or more enzymes within the BH4 biosynthetic pathway.

Enzymes within the pterin or BH4 biosynthetic pathway include: GTP cyclohydrolase I (GTPCH), 6-pyruvoyl-tetrahydropterin synthase (PTPS), sepiapterin reductase (SP), carbonyl reductase (CR), aldo-keto reductase (AKR), dihydrofolate reductase (DHFR), dihydropteridine reductase (DHPR), pterin-4a-carbinolamine dehydratase (PCD), and endothelial NOS (eNOS).

Suitably the present invention is for the prevention or treatment of a disorders associated with a pterin or BH4 deficiency caused by impaired activity of one or more of: GTP cyclohydrolase I (GTPCH), 6-pyruvoyl-tetrahydropterin synthase (PTPS), sepiapterin reductase (SP), carbonyl reductase (CR), aldo-keto reductase (AKR), dihydrofolate reductase (DHFR), dihydropteridine reductase (DHPR), pterin-4a-carbinolamine dehydratase (PCD), and endothelial NOS (eNOS).

Suitably the present invention is for the prevention or treatment of a pterin or BH4 deficiency caused by impaired activity of one or more of: GTP cyclohydrolase I (GTPCH), 6-pyruvoyl-tetrahydropterin synthase (PTPS), sepiapterin reductase (SP), carbonyl reductase (CR), aldo-keto reductase (AKR), dihydrofolate reductase (DHFR), dihydropteridine reductase (DHPR), pterin-4a-carbinolamine dehydratase (PCD), and endothelial NOS (eNOS). Suitably the present invention is for the prevention or treatment of a disorder caused by impaired activity of one or more of: GTP cyclohydrolase I (GTPCH), 6-pyruvoyl-tetrahydropterin synthase (PTPS), sepiapterin reductase (SP), carbonyl reductase (CR), aldo-keto reductase (AKR), dihydrofolate reductase (DHFR), dihydropteridine reductase (DHPR), pterin-4a-carbinolamine dehydratase (PCD), and endothelial NOS (eNOS).

Disorders caused by impaired activity of one or more of the enzymes in the pterin or BH4 biosynthetic pathway include: BH4 deficiency (Tetrahydrobiopterin deficiency), GTP cyclohydrolase deficiency, dopa-responsive dystonia, 6-pyruvoyl-tetrahydropterin synthase deficiency, sepiapterin reductase deficiency, dihydrofolate reductase deficiency, dihydropteridine reductase deficiency, and pterin-4a-carbinolamine dehydratase deficiency. Suitably, therefore, the present invention is for the prevention or treatment of any of these disorders.

In one embodiment, the present invention is for the prevention or treatment of a disorders associated with low BH4 bioavailability caused by impaired activity of GTP cyclohydrolase I (GTPCH), suitably GTP cyclohydrolase deficiency. In one embodiment, the present invention is for the prevention or treatment of low BH4 bioavailability caused by impaired activity of GTP cyclohydrolase I (GTPCH), suitably GTP cyclohydrolase deficiency. In one embodiment, the present invention is for the prevention or treatment of a disorder caused by impaired activity of GTP cyclohydrolase I (GTPCH), suitably GTP cyclohydrolase deficiency.

In one embodiment, the present invention is for the prevention or treatment of GTP cyclohydrolase deficiency.

Suitably, disorders associated with low BH4 bioavailability may also include disorders involving enzymes which depend on BH4 as a cofactor. Suitably therefore, disorders associated with low BH4 bioavailability may be substantially the same as disorders associated with biopterin-dependent enzymes.

Suitably disorders associated with low BH4 bioavailability may include disorders associated with biopterin-dependent enzymes. Suitably the present invention is for the prevention or treatment of a disorders associated with impaired activity of one or more biopterin-dependent enzymes.

Suitably disorders associated with low BH4 bioavailability may include disorders associated with biopterin-dependent hydroxylases or synthases. Suitable biopterin-dependent hydroxylases or synthases may be selected from phenylalanine hydroxylase, tyrosine hydroxylase, tryptophan hydroxylase 1, tryptophan hydroxylase 2, and nitric oxide synthase.

Suitably, disorders associated with low BH4 bioavailability may be selected from disorders associated with or caused by impaired activity of phenylalanine hydroxylase, tyrosine hydroxylase, tryptophan hydroxylase 1 or 2, or nitric oxide synthase.

Suitably, disorders associated with or caused by impaired activity of phenylalanine hydroxylase include phenylketonuria (PKU), cirrhosis, and fatty liver disease.

Suitably, disorders associated with or caused by impaired activity of tyrosine hydroxylase include: Tyrosine hydroxylase deficiency, Segawa's dystonia, Parkinson's disease, infantile parkinsonism, DOPA-responsive dystonia, schizophrenia, Alzheimer's disease, bipolar disorder, autism, ADHD, and depression.

Suitably, disorders associated with or caused by impaired activity of tryptophan hydroxylase 1 or 2 include osteoporosis, hypertension, ADHD, schizophrenia, autism, depression, bipolar disorder, personality disorders.

Suitably, disorders associated with or caused by impaired activity of nitric oxide synthase may include disorders associated with or caused by impaired activity of neuronal nitric oxide synthase, (nNOS or NOS1), endothelial NOS (eNOS or NOS3), or inducible NOS (iNOS or NOS2). Suitably, disorders associated with or caused by impaired activity of a nitric oxide synthase may include depression, bipolar disorder, stroke, Parkinson's disease, Alzheimer's disease, amytrophic lateral sclerosis, diabetes, myocardial hypertrophy, cardiomyopathy, hypertension, atherosclerosis, ischaemia-reperfusion, pregnancy-induced hypertension, placental insufficiency, foetal growth restriction, and pre-eclampsia.

In one embodiment, the present invention is for the prevention or treatment of a disorder associated with low BH4 bioavailability caused by impaired activity of phenylalanine-4 hydroxylase (PAH), suitably phenylketonuria (PKU). In one embodiment, the present invention is for the prevention or treatment of a disorder caused by impaired activity of phenylalanine-4 hydroxylase (PAH), suitably phenylketonuria (PKU).

In one embodiment, the present invention is for the prevention or treatment of a disorder associated with low BH4 bioavailability caused by impaired activity of endothelial nitric oxide synthase, suitably pre-eclampsia. In one embodiment, the present invention is for the prevention or treatment of a disorder caused by impaired activity of endothelial nitric oxide synthase, suitably pre-eclampsia.

Suitably the impaired activity of any of the above biopterin dependent enzymes is caused by low BH4 bioavailability. Suitably therefore, the impaired activity may be BH4-mediated impaired activity. Suitably any of the above disorders or diseases is caused by low BH4 bioavailability. Suitably therefore, the diseases or disorders may be associated with or derived from low BH4 bioavailability. Suitably the low BH4 bioavailability may itself be caused by a BH4 deficiency.

Suitably the present invention may be for the prevention or treatment of a cardiac disease or disorder, a liver disease or disorder, a neurological disease or disorder, or a vascular disease or disorder associated with low BH4 bioavailability.

Suitable cardiac diseases associated with low BH4 bioavailability include: diabetes, myocardial hypertrophy, cardiomyopathy, and ischaemia-reperfusion.

Suitable liver diseases associated with low BH4 bioavailability include: metabolic disorders such as phenylketonuria, cirrhosis and fatty liver disease.

Suitable neurological diseases associated with low BH4 bioavailability include: autism, ADHD, Parkinson's disease, neuropathy, amyotrophic lateral sclerosis, dystonia, depression, Alzheimer's disease, and psychiatric conditions such as schizophrenia, bipolar disorder, personality disorder.

Suitable vascular diseases associated with low BH4 bioavailability include: hypertension, atherosclerosis, stroke, pregnancy-induced hypertension, placental insufficiency, foetal growth restriction, and pre-eclampsia.

Suitably the present invention may be for the prevention or treatment of a pregnancy-related disorder. Suitably the present invention may be for the prevention or treatment of a pregnancy-related disorder associated with low BH4 bioavailability.

In one embodiment, the disorder associated with low BH4 bioavailability is a vascular disease. In one embodiment, the disorder associated with low BH4 bioavailability is an endothelial disorder. In one embodiment, the disorder associated with low BH4 bioavailability is a pregnancy-related vascular disease. In one embodiment, the disorder associated with low BH4 bioavailability is a pregnancy-related endothelial disorder.

In one preferred embodiment, the disorder associated with low BH4 bioavailability is pre-eclampsia, therefore the invention is for the treatment or prevention of pre-eclampsia.

In one embodiment, the disorder associated with low BH4 bioavailability may be a recurrent disorder. In one embodiment, the pre-eclampsia may be recurrent. Suitably recurrent pre-eclampsia occurs in woman who have experienced pre-eclampsia before in prior pregnancies.

### Prevention or Treatment

The present invention relates to the use of a reduced folate optionally in combination with BH4, a precursor, or a functional equivalent thereof in the prevention or treatment of a disorder associated with low BH4 bioavailability.

As noted above, ‘treatment’ may suitably refer to any of: alleviation of symptoms, diminishment of extent of disease, stabilized (i.e., not worsening) state of disease, delay or slowing of disease progression, amelioration or palliation of the disease state, and remission (whether partial or total), whether detectable or undetectable.

‘Prevention’ may additionally refer to avoiding a disease, or a symptom of a disease.

Suitably, the reduced folate optionally in combination with BH4, a precursor, or a functional equivalent thereof, normalises or restores BH4 levels in a subject. Suitably, the reduced folate optionally in combination with BH4, a precursor, or a functional equivalent thereof increases BH4 levels in a subject. In one embodiment, the BH4 levels are measured in endothelial cells. Suitably, the reduced folate optionally in combination with BH4, a precursor, or a functional equivalent thereof, normalises or restores BH4 levels in endothelial cells of a subject. Suitably, the reduced folate optionally in combination with BH4, a precursor, or a functional equivalent thereof increases BH4 levels in endothelial cells of a subject.

Suitably, the reduced folate optionally in combination with BH4, a precursor, or a functional equivalent thereof, reduces or prevents oxidation of BH4 in a subject. Suitably, therefore the reduced folate prevents loss of BH4 by oxidation. Suitably the addition of BH4 in combination therewith may further directly increase the BH4 levels in a subject. Suitably the reduced folate may also increase the level of BH4 by other mechanisms. Suitably the reduced folate may interact with dihydrofolate reductase (DHFR) to increase the levels, stability or activity of this enzyme. Suitably an increase in activity of DHFR increases reduction of BH2 back into BH4. Suitably this may be a direct interaction with DHFR, or an indirect interaction. Suitably the reduced folate may block the effects of a DHFR inhibitor, such as methotrexate. Suitably the reduced folate may act to increase GTP cyclohydrolase I (GTPCH) expression or activity, suitably endothelial GTPCH expression or activity.

Suitably the reduced folate may have other intracellular redox effects to increase cellular reducing capacity. Suitably an increase in cellular reducing capacity may comprise an increase in the levels of cellular reducing agents or systems such as those associated with glutathione, NADPH or peroxyredoxins. Suitably the reduced folate may scavenge or inhibit the actions of reactive oxygen species (ROS) or reactive nitrogen species (RNS). Suitably therefore, the reduced folate may reduce or modify the biological effects of ROS or RNS on target molecules, either directly or via effects on cellular reducing agents or systems. Suitably the reduced folate may act to increase nitric oxide synthase expression or activity, suitably endothelial nitric oxide synthase expression or activity. Suitably the reduced folate may act to reduce reactive oxygen species, suitably to reduce reactive oxygen species in endothelial cells.

Suitably a reduced folate optionally in combination with BH4, a precursor, or a functional equivalent thereof prevents or treats a disorder associated with low BH4 bioavailability by maintaining or restoring a healthy BH4 level in a subject.

Suitably a reduced folate optionally in combination with BH4, a precursor, or a functional equivalent thereof prevents or treats a disorder associated with low BH4 bioavailability by increasing the level of BH4 in a subject. Suitably by increasing the level of BH4 in a subject to a healthy BH4 level.

Suitably by healthy BH4 level it is meant the level of BH4 in a reference healthy subject.

Suitably the reduced folate optionally in combination with BH4, a precursor, or a functional equivalent thereof increases the level of BH4 in a subject by reducing or preventing oxidation of BH4. Suitably by reducing or preventing oxidation of BH4 into BH2.

Suitably therefore, a reduced folate optionally in combination with BH4, a precursor, or a functional equivalent thereof prevents or treats a disorder associated with low BH4 bioavailability by reducing or preventing oxidation of BH4.

Suitably reduced folate optionally in combination with BH4, a precursor, or a functional equivalent can increase BH4 levels in a subject by up to 500%, suitably by up to 400%, suitably by up to 300%, suitably by up to 200%, suitably by up to 100%, 50%, suitably by 45%, suitably by 40%, suitably by 35%, suitably by 30%, suitably by 25%.

Suitably reduced folate optionally in combination with BH4, a precursor, or a functional equivalent can improve the oxidation status of BH4 in a subject by up to 500%, suitably by up to 400%, suitably by up to 300%, suitably by up to 200%, suitably by up to 100%, suitably by up to 50%, suitably by up to 25%. Suitably by ‘oxidation status of BH4 in a subject’ it is meant the level of BH4 relative to BH2 or other oxidised forms of biopterins or pterins in the subject.

Suitably reduced folate optionally in combination with BH4, a precursor, or a functional equivalent can increase the ratio of BH4:BH2 by up to 14 fold, suitably up to 12 fold, suitably up to 10 fold, suitably up to 8 fold, suitably up to 6 fold, suitably up to 4 fold, suitably up to 2 fold. Suitably reduced folate optionally in combination with BH4, a precursor, or a functional equivalent can increase the ratio of BH4:BH2 to between 1.0 and 1.5.

Suitably the increases are compared to a control. A suitable control is the relevant measurement in a subject having the same condition or disease who has not received any treatment with a reduced folate optionally in combination with BH4. For example, the level of BH4 in a subject who has a disorder associated with low BH4 bioavailability and who has not received any treatment with a reduced folate optionally in combination with BH4.

### Sole API

In some aspects of the present invention, a disorder associated with low BH4 bioavailability may be prevented or treated solely with a reduced folate. The present inventors have discovered that providing a reduced folate alone can treat or prevent disorders associated with low BH4 bioavailability without needing to provide BH4 itself or a precursor or functional equivalent thereof.

Suitably, the reduced folate may be the sole active pharmaceutical ingredient (API). Suitably, the reduced folate may be used alone. Suitably, the reduced folate may not be used in combination with any other active pharmaceutical ingredient. Suitably a subject may be provided solely with reduced folate for the treatment or prevention of a disorder associated with low BH4 bioavailability. Suitably a subject may be treated solely with reduced folate in respect of a disorder associated with low BH4 bioavailability. Suitably no other active pharmaceutical ingredient may be provided or administered to a subject in respect of treating or preventing a disorder associated with low BH4 bioavailability. Suitably, no other active pharmaceutical ingredient may be used to treat a subject in respect of a disorder associated with low BH4 bioavailability.

‘Active pharmaceutical ingredient’ as used herein is intended to mean any substance which provides a pharmacological effect in the human body. The term does not include inert substances that have no pharmacological effect in the human body such as excipients. Suitable excipients are defined hereinbelow.

Suitably therefore, the aspects of the present invention which refer to use of reduced folate as the sole active pharmaceutical ingredient, may include the use of excipients. Suitably excipients may be included with the reduced folate. Suitably, in such cases, the reduced folate is comprised in a composition, wherein the composition may comprise excipients. Suitably therefore, the invention may comprise treatment or prevention with a composition comprising one or more active pharmaceutical ingredients, and one or more excipients, wherein the active pharmaceutical ingredients consist of a reduced folate. Suitably the composition may be a pharmaceutical composition.

In one embodiment of the third aspect of the present invention, there is provided a composition comprising one or more active pharmaceutical ingredients, and one or more excipients, for use in the prevention or treatment of a disorder associated with low BH4 bioavailability, wherein the active pharmaceutical ingredient consists of a reduced folate.

### Combination Therapy

In some aspects of the invention, the reduced folate may be used in combination with other active pharmaceutical ingredients. For example, the reduced folate may be used in combination with BH4, a precursor, or functional equivalent thereof as described hereinbelow, but it may also be combined with other active pharmaceutical ingredients.

Suitably the other active pharmaceutical ingredients may include any other pharmaceutical ingredient which has a beneficial effect on disorders associated with low BH4 bioavailability. Suitable other active pharmaceutical ingredients may include a further reduced folate and/or a further BH4 precursor or functional equivalent thereof. Suitable other active pharmaceutical ingredients may also include: vitamins such as vitamin C and/or vitamin B12; and/or neurotransmitter precursors such as L-DOPA or carbidopa; and/or 5-hydroxytryptophan; and/or arginine and/or citrulline.

However, suitably, the other active pharmaceutical ingredients do not include aspirin, or a herbal extract. Suitably the reduced folate is not for administration in combination with aspirin, or a herbal extract.

Suitably the reduced folate may not be for administration in combination with a salicylate. Suitably therefore the reduced folate may not be combined with a salicylate.

‘Salicylate’ as used herein means any drug which is derived from salicylic acid. Suitably such drugs are NSAIDs. Examples of salicylates include: aspirin, diflunisal, salicylic acid, and salsalate.

Suitably the reduced folate may not be for administration in combination with a non-steroidal anti-inflammatory drug (NSAID). Suitably therefore the reduced folate may not be combined with a non-steroidal anti-inflammatory drug (NSAID).

‘Non-steroidal anti-inflammatory drug’ (NSAID) as used herein means any drug which reduces inflammation and/or pain but which is not a steroid. Suitably an NSAID may include any drug which inhibits the activity of a cyclooxygenase enzyme, suitably COX1 and/or COX2. Examples of NSAIDs include: aspirin, diflunisal, salicylic acid, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone, mefenamic acid, meclofenamic acid, flufenamic acid, tolfenamic acid, celecoxib, rofecoxib, valdecoxib, parecoxib, lumiracoxib, etoricoxib, firocoxib, nimesulide, clonixin, and licofelone.

‘Herbal extract’ as used herein means any compound or mixture thereof obtained after using a solvent to select for, or remove, components of a plant or any part of a plant. A herbal extract may be in a dry, liquid or semisolid form. Herbal extracts include any Chinese herbal medicine. Suitably the reduced folate may not be for administration in combination with a Chinese herbal extract or medicine. Examples of herbal extracts include: quince extract, euryale extract fringed pink extract, honeysuckle extract, houttuynia extract, citron extract, lotus extract, lesser galangal extract, chrysanthemum extract, mint extract, sophora extract, rice bean extract, wheat extract, and Japanese thistle extract.

In some aspects of the present invention, a disorder associated with low BH4 bioavailability may be prevented or treated with a reduced folate in combination with BH4, a precursor, or functional equivalent thereof. The addition of BH4, a precursor, or functional equivalent thereof with the reduced folate may aid in increasing the bioavailability of BH4 directly. Suitably this may be especially useful for treating subjects having low BH4 levels, or a lack of BH4.

Suitably, the reduced folate and BH4, a precursor, or functional equivalent thereof may be the only active pharmaceutical ingredients for use in the treatment. Suitably, reduced folate and BH4, a precursor, or functional equivalent thereof may be used in combination without any other active pharmaceutical ingredient. Suitably a subject may be provided solely with reduced folate and BH4, a precursor, or functional equivalent thereof for the treatment or prevention of a disorder associated with low BH4 bioavailability. Suitably no other active pharmaceutical ingredient may be provided or administered to the subject in respect of treating or preventing a disorder associated with low BH4 bioavailability. Suitably, no other active pharmaceutical ingredient may be used to treat the subject in respect of a disorder associated with low BH4 bioavailability.

Suitably therefore, the aspects of the present invention which refer to use of reduced folate and BH4, a precursor, or functional equivalent as the only active pharmaceutical ingredients, may still include the use of excipients. Suitably excipients may be included with the reduced folate and BH4, a precursor, or functional equivalent. Suitably, in such cases, the reduced folate BH4, a precursor, or functional equivalent is comprised in a composition, wherein the composition may comprise excipients. Suitably therefore, the invention may comprise treatment or prevention with a composition comprising one or more active pharmaceutical ingredients, and one or more excipients, wherein the active pharmaceutical ingredients consist of a reduced folate and BH4, a precursor, or functional equivalent thereof.

In one embodiment of the third aspect of the present invention, there is provided a composition comprising one or more active pharmaceutical ingredients, and one or more excipients, for use in the prevention or treatment of a disorder associated with low BH4 bioavailability, wherein the active pharmaceutical ingredients consist of a reduced folate and BH4, a precursor, or functional equivalent thereof.

The combination of reduced folate and BH4, a precursor, or functional equivalent thereof may be administered in any suitable way. Suitably the reduced folate and BH4, a precursor, or functional equivalent may be administered simultaneously, or sequentially in any order, to a subject.

Suitably, sequential administration comprises administering a first active pharmaceutical ingredient and subsequently administering a second active pharmaceutical ingredient, suitably after an interval of time.

Suitably, if the reduced folate and BH4, a precursor, or functional equivalent are administered sequentially, the reduced folate may be the first active pharmaceutical ingredient and the BH4, a precursor, or functional equivalent may be the second active pharmaceutical ingredient. Alternatively, the BH4, a precursor, or functional equivalent may be the first active pharmaceutical ingredient, and the reduced folate may be the second pharmaceutical ingredient.

Suitably sequential administration may comprise an interval of time between administering the first active pharmaceutical ingredient and a second active pharmaceutical ingredient. Suitably the interval of time may be seconds, minutes, hours, days, or weeks. Suitably the interval of time may be, for example, 30 seconds, 1 minute, 2 minutes, 5 minutes, 10 minutes, 20 minutes, 30 minutes, 1 hour, 2 hours, 4 hours, 8 hours, 24 hours, 48 hours, 4 days, 7 days, 10 days, 14 days.

Suitably the interval of time is selected such that both APIs are able to exert their therapeutic effect at overlapping time periods.

Suitably, the reduced folate and BH4, a precursor, or functional equivalent thereof may be formulated together in a single composition. Alternatively, the reduced folate and BH4, a precursor, or functional equivalent thereof may be formulated in two separate compositions.

Suitably, for sequential administration, the reduced folate and BH4, a precursor, or functional equivalent thereof are formulated in two separate compositions. Suitably a first composition and a second composition. Suitably the first composition comprises the first active pharmaceutical ingredient and the second composition comprises the second active pharmaceutical ingredient.

Suitably, for simultaneous administration, the reduced folate and BH4, a precursor, or functional equivalent thereof may be formulated in a single composition, or may be formulated into two separate compositions, suitably into two separate compositions that are administered together.

Suitably the first and second compositions may comprise different formulations. Suitable formulations for pharmaceutical compositions are described elsewhere herein.

In one embodiment, the reduced folate and BH4, a precursor, or functional equivalent are administered simultaneously in a single composition.

Suitably the active pharmaceutical ingredients or compositions comprising them are administered to a subject via a suitable route of administration. Suitable routes of administration are described hereinbelow. Suitably the first and second active pharmaceutical ingredients or first and second compositions comprising them may be administered by different routes.

For example, the first active pharmaceutical ingredient or composition thereof may be administered intravenously, and the second active pharmaceutical ingredient or composition thereof may be administered orally.

In one embodiment, the reduced folate and BH4, a precursor, or functional equivalent are administered simultaneously in a single composition by the same route of administration. In one embodiment, the reduced folate and BH4, a precursor, or functional equivalent are administered simultaneously in a single composition by oral administration.

### Pharmaceutical Composition

The reduced folate, and optionally the BH4 or a precursor, or functional equivalent thereof used in the aspects of the present invention may be formulated into one or more compositions for administration to a subject. Suitably the compositions are pharmaceutical compositions.

Suitably any reference herein to a ‘composition’ or an ‘active pharmaceutical ingredient’ for use according to the invention may also refer to a pharmaceutical composition, suitably a pharmaceutical composition comprising an active pharmaceutical ingredient for use according to the invention.

Suitably, a pharmaceutical composition comprises one or more active pharmaceutical ingredients (API) and one or more excipients, diluents and/or carriers. Suitably, a pharmaceutical composition for use in the present invention may comprise a reduced folate and optionally BH4 or a precursor, or functional equivalent thereof, and one or more excipients, diluents and/or carriers.

According to an eleventh aspect of the present invention there is provided a pharmaceutical composition comprising a reduced folate and BH4, a precursor, or functional equivalent thereof.

Suitably the pharmaceutical composition may further comprise one or more excipients, diluents and/or carriers. Excipients may be added in order to adjust the concentration; enhance stability; limit microbial growth; to improve drying, flow, or other manufacturing characteristics of the composition; to enhance bioavailability; or to slow down, or speed up absorption of the API; for example.

Suitably, a pharmaceutical composition for use in the present invention may comprise one or more active pharmaceutical ingredients which consist of a reduced folate and optionally BH4 or a precursor, or functional equivalent thereof, and one or more further excipients, diluents and/or carriers. In one embodiment, a pharmaceutical composition for use in the present invention may comprise a sole active pharmaceutical ingredient and one or more excipients, diluents and/or carriers, wherein the sole active pharmaceutical ingredient is reduced folate.

Suitably, as described hereinabove, the active pharmaceutical ingredients of a reduced folate and BH4 or a precursor, or functional equivalent thereof may be comprised in two different pharmaceutical compositions. Suitably the first pharmaceutical composition comprises the first active pharmaceutical ingredient and the second pharmaceutical composition comprises the second active pharmaceutical ingredient. Suitably the first pharmaceutical composition may comprise reduced folate, and the second pharmaceutical composition may comprise BH4 or a precursor, or functional equivalent thereof, or vice versa. Suitably the first pharmaceutical composition may comprise a sole active pharmaceutical ingredient of reduced folate, and the second pharmaceutical composition may comprise a sole active pharmaceutical ingredient of BH4 or a precursor, or functional equivalent thereof, or vice versa.

Suitably the first and second pharmaceutical compositions may comprise different formulations. Suitably the formulations are tailored to the active pharmaceutical ingredient contained therein, to maximise efficacy of the active pharmaceutical ingredient.

Suitably, the optimal pharmaceutical formulation can be determined by one of skill in the art depending on the route of administration and the desired dosage. See for example Remington's Pharmaceutical Sciences, 18th Ed. (1990, Mack Publ. Co, Easton Pa. 18042) pp 1435 1712.

Suitable excipients, carriers or diluents may include, for example, a binder, disintegration agent, surfactant, flavouring, coating material, preservative, colouring, thickener, antimicrobial agent, antioxidant, or lubricant, or combinations thereof.

Nonlimiting examples of binders include gum tragacanth, acacia, starch, gelatine, and biological degradable polymers such as homo- or co-polyesters of dicarboxylic acids, alkylene glycols, polyalkylene glycols and/or aliphatic hydroxyl carboxylic acids; homo- or co-polyamides of dicarboxylic acids, alkylene diamines, and/or aliphatic amino carboxylic acids; corresponding polyester-polyamide-co-polymers, polyanhydrides, polyorthoesters, polyphosphazene and polycarbonates. The biological degradable polymers may be linear, branched or crosslinked. Specific examples are poly-glycolic acid, poly-lactic acid, and poly-d,l-lactide/glycolide. Other examples for polymers are water-soluble polymers such as polyoxaalkylenes (polyoxaethylene, polyoxapropylene) and mixed polymers thereof, poly-acrylamides and hydroxylalkylated polyacrylamides, poly-maleic acid and esters or-amides thereof, poly-acrylic acid and esters or-amides thereof, poly-vinylalcohol und esters or-ethers thereof, poly-vinylimidazole, poly-vinylpyrrolidon, und natural polymers like chitosan. Exemplary disintegration agents include polyvinylpyrrolidone (PVP, e.g. sold under the name POVIDONE), a cross-linked form of povidone (CPVP, e.g. sold under the name CROSPOVIDONE), a cross-linked form of sodium carboxymethylcellulose (NaCMC, e.g. sold under the name AC-DI-SOL), other modified celluloses, and modified starch.

Exemplary antioxidants include ascorbic acid, fatty acid esters of ascorbic acid such as ascorbyl palmitate and ascorbyl stearate, and salts of ascorbic acid such as sodium, calcium, or potassium ascorbate. Non-acidic antioxidants may also be used such as beta-carotene, alpha-tocopherol.

Nonlimiting examples of lubricants include natural or synthetic oils, fats, waxes, or fatty acid salts such as magnesium stearate.

Exemplary surfactants can be anionic, anionic, amphoteric or neutral. Nonlimiting examples of surfactants include lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono-or-distearate, glycerol mono-or-dioleate and glycerol mono-or-dipalmitate, and polyoxyethylene stearate.

Nonlimiting examples of preservatives include methyl or propylparabens, sorbic acid, chlorobutanol, phenol and thimerosal.

In one embodiment, a pharmaceutical composition comprising a reduced folate and BH4, a precursor, or functional equivalent thereof comprises the following excipients, diluents and/or carriers: anhydrous dibasic calcium phosphate, crospovidone, and stearyl fumarate.

Suitably the pharmaceutical composition may be formulated as a solid or a liquid. Suitably the pharmaceutical composition is formulated as a solid selected from a pill, capsule, tablet or powder. In one embodiment, the pharmaceutical composition is formulated as a tablet. Suitably the solid formulation is water-soluble.

Suitably a pharmaceutical composition may be administered to a subject by any suitable route. Suitable routes of administration may be: oral, parenteral, by inhalation or topical.

Suitably, the term parenteral as used herein includes, e.g., intravenous, intraarterial, intraperitoneal, intramuscular, subcutaneous, rectal, or vaginal administration.

In one embodiment, the compositions of the invention are administered orally. In one embodiment, the compositions of the invention are comprised in the form of a tablet which is administered orally.

Suitably the active pharmaceutical ingredients for use in the invention or the compositions comprising the active pharmaceutical ingredients for use in the invention may be administered to a subject in a pharmaceutically effective amount. Suitably a pharmaceutically effective amount is an amount of the or each active pharmaceutical ingredient which is sufficient to achieve a therapeutic effect. Suitably the pharmaceutically effective amount may be an amount of the or each active pharmaceutical ingredient which is effective to ameliorate a disorder associated with low BH4 bioavailability. Suitably the pharmaceutically effective amount may be an amount of the or each active pharmaceutical ingredient which is effective to achieve an increase in BH4 levels in a subject.

Suitably a single dose of the active pharmaceutical ingredients for use in the present invention comprises a pharmaceutically effective amount.

Suitable doses of the active pharmaceutical ingredients for use in the present invention can be determined by a person skilled in the art.

However a suitable dose for BH4, a precursor, or a functional equivalent thereof may be, for example: between 1 to about 20 mg/kg body weight per day, suitably between 10 mg/kg to about 20 mg/kg per day. Suitably the daily dose may be 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg per day. Suitably such a dose may be used in a pharmaceutical composition comprising the BH4, precursor, or functional equivalent thereof. Suitably such a dose may be used in a pharmaceutical composition comprising the BH4, precursor, or functional equivalent thereof, and further comprising one or more additional active ingredients, such as a reduced folate.

A suitable dose for a reduced folate may be between 1 mg and 50 mg, suitably between 2 mg and 30 mg, suitably between 3 mg and 25 mg, suitably between 4 mg and 20 mg, suitably between 5 mg and 15 mg per day. Suitably such a dose may be used in a pharmaceutical composition comprising the reduced folate. Suitably such a dose may be used in a pharmaceutical composition comprising the reduced folate, and further comprising one or more additional active ingredients, such as BH4, a precursor, or functional equivalent thereof.

The suitable dose of an active pharmaceutical ingredient of the invention or a composition according to the present invention will depend upon the sex, age, health, height, diet, and weight of the recipient, any concurrent treatment, and the nature of the effect desired. The most preferred dosage can be tailored to the individual subject, as is understood and determinable by one of skill in the art, without undue experimentation. This typically involves adjustment of a standard dose, e.g., reduction of the dose if the patient has a low body weight.

Suitably the active pharmaceutical ingredients of the invention or the compositions comprising the active pharmaceutical ingredients of the invention may be administered in multiple doses or in a single dose. Suitably multiple doses may be administered at suitable intervals. Suitably, dosage regimens also may be adjusted to provide the optimum desired response (e.g., a therapeutic or preventative response).

### Subject

The present invention is for use in the prevention or treatment of a disorder associated with low BH4 bioavailability in a subject.

Suitably the subject is a mammal. Suitably the subject is human. Suitably the subject may be an adult or a child. Suitably the subject may be male or female. In some embodiments, wherein the disorder is a pregnancy related disorder, the subject is female. In such embodiments, the subject is a pregnant female.

Suitably the aspects of the invention may be for prevention or treatment of a disorder associated with low BH4 bioavailability in a subject in need thereof.

A subject in need thereof may be a subject that has been diagnosed with a disorder associated with low BH4 bioavailability, or a subject suspected of having a disorder associated with low BH4 bioavailability. Suitable disorders are defined herein elsewhere.

A subject diagnosed with having a disorder associated with low BH4 bioavailability may have undergone diagnostic testing. Suitably the subject may have been tested for one or more markers of a disorder associated with low BH4 bioavailability, for example a reduced BH4 level as described further hereinbelow. Suitably the subject may have been tested in accordance with the tenth aspect of the present invention. Alternatively, or additionally, the subject may have been tested for reduced activity of one or more enzymes in the pterin biosynthetic pathway as described elsewhere herein, or one or more biopterin dependent enzymes as described elsewhere herein.

In one embodiment, the subject has been diagnosed with pre-eclampsia. Suitably the subject may have been diagnosed with pre-eclampsia by testing for hypertension; thrombocytopenia; and/or proteinuria.

Suitably, a subject that has been diagnosed with, or is known to have, a disorder may be known as a ‘patient’ herein.

A subject suspected of having a disorder associated with low BH4 bioavailability may display one or more symptoms of a disorder associated with low BH4 bioavailability. Symptoms may include: headaches; temporary low muscle tone; intellectual disability; mood swings; low mood; suicidal thoughts; psychosis; outbursts; insomnia; hallucinations; loss of vision; delusions; memory loss; confusion; language difficulties; movement disorders including rigidity; dystonia or tremor; difficulty swallowing; acid reflux; nausea; vomiting; pain; weight gain; oedema; seizures; behavioural problems; progressive problems with development; inability to control body temperature; hyperactivity; metabolic derangements including disturbance of amino acid levels; cardiovascular abnormalities; microcephaly; low birth weight; hypertension; thrombocytopenia; impaired liver function; kidney dysfunction; swelling; bruising; shortness of breath; proteinuria. In one embodiment, the subject has one or more of these symptoms.

A subject suspected of having a disorder associated with low BH4 bioavailability may have one or more markers of a disorder associated with low BH4 bioavailability. Suitable markers associated with low BH4 bioavailability include:
(i) A low level of BH4;
(ii) A low ratio of BH4 to BH2;
(iii) A low ratio of BH4 to total biopterins;
(iv) A low level of reduced folates
(v) A low level of reduced folates relative to oxidised folates;
(vi) Reduced forearm blood flow responses;
(vii) Reduced flow-mediated vasodilation;
(viii) Increased circulating sICAM, P-selectin, von Willebrand factor, or microalbuminuria;
(ix) A low level of a neurotransmitter (such as serotonin, dopamine, or their metabolites);
(x) Increased level of phenylalanine or related metabolites; and/or
(xi) A high ratio of sFlt-1 to PIGF
(xii) A disturbed folate metabolism due to e.g. a defect in homocysteine methyltransferase or a deficiency of Vitamin B12
(xiii) A high plasma homocysteine level (optionally due to either vitamin B-12 or folate deficiency)
(xiv) A high methylmalonic acid level (optionally due to vitamin B-12 deficiency)

Suitably relative terms such as ‘low’, ‘high’, ‘increased’, or ‘reduced’ are relative to the same marker when measured in a reference subject.

In one embodiment, the subject has one or more of these markers. In one embodiment, the subject has one or more of the above symptoms and/or one or more of the markers.

In one embodiment, the subject may display one or more symptoms of pre-eclampsia selected from: headaches, impaired vision, acid reflux, pain below the ribs, nausea or vomiting, hypertension; thrombocytopenia; impaired liver function; kidney dysfunction; swelling; bruising; shortness of breath; heartburn; pain below the ribs; excessive weight gain; sudden increase in oedema; and proteinuria.

Suitably hypertension may be defined as blood pressure of ≥140 mmHg systolic or ≥90 mmHg diastolic on at least two separate occasions.

Suitably thrombocytopenia may be defined as a platelet count <100,000/microliter of blood.

Suitably proteinuria may be defined as ≥0.3 grams (300 mg) or more of protein in a 24-hour urine sample, or a SPOT urinary protein to creatinine ratio ≥0.3, or a urine dipstick reading of 1+ or greater.

In one embodiment, a subject suspected of having pre-eclampsia may have a high sFlt-1/PIGF ratio. Suitably a high sFlt-1/PIGF ratio may be defined as higher than 38. Suitably a sFlt-1/PIGF ratio of higher than 85 in early pregnancy (which may be up to 34 weeks), or higher than 110 in late pregnancy (which may be after 34 weeks) indicates a high risk of having pre-eclampsia.

In one embodiment, the subject is a pregnant woman having hypertension, proteinuria and/or thrombocytopenia. In one embodiment the subject is suspected of having pre-eclampsia.

In various aspects and embodiments of the invention, comparison of the subject is made to a reference subject. Suitably the reference subject is a healthy subject. Suitably a subject who does not have a disorder associated with low BH4 bioavailability. Suitably the reference subject is equivalent to the subject. Suitably the reference subject should be equivalent in terms of sex, age, weight, and ethnicity as the subject.

### Dosage Regimen

Suitably the active pharmaceutical ingredients of the invention or the compositions comprising the active pharmaceutical ingredients of the invention may be administered according to a defined dosage regimen.

Suitably the active pharmaceutical ingredients of the invention or the compositions thereof may be administered multiple times per day, daily, every two days, every four days, once per week, once every 10 days, or once every 2 weeks, for example. In one embodiment, the active pharmaceutical ingredients of the invention or the compositions comprising the active pharmaceutical ingredients of the invention are administered once or twice daily.

Suitably the reduced folate or the pharmaceutical compositions comprising the reduced folate are administered once or twice per day. Suitably the BH4, precursor, or functional equivalent thereof, or the pharmaceutical compositions comprising the BH4, precursor, or functional equivalent thereof are administered once or twice per day.

Suitably the active pharmaceutical ingredients of the invention or the compositions comprising the active pharmaceutical ingredients of the invention may be administered during a defined therapeutic window.

Suitably the therapeutic window is selected to benefit the prevention or treatment of the relevant disorder associated with low BH4 bioavailability. Suitably the therapeutic window may be pre-disorder, during the disorder, or post-disorder. Suitably, therefore, active pharmaceutical ingredients of the invention or the compositions comprising the active pharmaceutical ingredients of the invention may be administered prior to a disorder associated with low BH4 bioavailability, during a disorder associated with low BH4 bioavailability, or after a disorder associated with low BH4 bioavailability. Suitably the therapeutic window may be before (planned) pregnancy, or during an early, mid or late stage of the relevant disorder. Suitably, therefore, active pharmaceutical ingredients of the invention or the compositions comprising the active pharmaceutical ingredients of the invention may be administered before (planned) pregnancy, or at an early, mid or late stage of a disorder associated with low BH4 bioavailability.

Suitably, when the relevant disorder associated with low BH4 bioavailability is a pregnancy-related disorder, the active pharmaceutical ingredients of the invention or the compositions comprising the active pharmaceutical ingredients of the invention may be administered pre-pregnancy, during pregnancy, or post-pregnancy. Suitably during pregnancy, the active pharmaceutical ingredients of the invention or the compositions comprising the active pharmaceutical ingredients of the invention may be administered during the first (0-13 weeks), second (14-26 weeks), or third (27-40 weeks) trimester of pregnancy.

Suitably, in the prevention of pregnancy-related disorders, the active pharmaceutical ingredients of the invention or the compositions comprising the active pharmaceutical ingredients of the invention are administered pre-pregnancy or during pregnancy.

Suitably, in the treatment of pregnancy-related disorders, the active pharmaceutical ingredients of the invention or the compositions comprising the active pharmaceutical ingredients of the invention are administered during pregnancy, or post-pregnancy.

Suitably, in some cases, the active pharmaceutical ingredients of the invention or the compositions comprising the active pharmaceutical ingredients of the invention are administered post-pregnancy to accelerate recovery and/or prevent long-term complications of pregnancy-related disorders. Suitably such disorders may be termed post-partum disorders.

In one embodiment, in the treatment of pre-eclampsia, suitably the active pharmaceutical ingredients of the invention or the compositions comprising the active pharmaceutical ingredients of the invention are administered when the condition arises. Typically this may be in late pregnancy, suitably after the 20^{th} week of pregnancy, suitably after the 22^{nd} week of pregnancy, suitably after the 24^{th} week of pregnancy, suitably during the third trimester.

However pre-eclampsia may also develop post-partum. Suitably the active pharmaceutical ingredients of the invention or the compositions comprising the active pharmaceutical ingredients of the invention may be administered post-partum. Suitably up to 6 weeks post-partum.

Method of Selecting a Subject that May Benefit from Treatment

In some aspects of the present invention, a subject may have been tested for one or more markers of a disorder associated with low BH4 bioavailability, in order to allow subjects to be selected that may have a disorder associated with a low BH4 bioavailability. These subjects can then be treated with the reduced folate and optionally BH4 or precursor or functional equivalent thereof. In addition or alternatively, a subject may be tested for one or more symptoms of a disorder associated with low BH4 bioavailability in order to allow subjects to be selected that may have a disorder associated with a low BH4 bioavailability. These subjects can then be treated with the reduced folate and optionally BH4 or precursor or functional equivalent thereof.

Suitably the subject may be tested by the method of the tenth aspect.

Suitably subjects who are determined to have the one or more markers and/or symptoms may be diagnosed with having a disorder associated with a BH4 deficiency. Suitably therefore the invention may further provide a method of diagnosing a subject as having a disorder associated with low BH4 bioavailability. Suitably comprising the steps of testing as referred to herein. Additionally, or alternatively, subjects who are determined to have the one or more markers and/or symptoms may be selected for treatment, suitably with a reduced folate and optionally BH4 or precursor or functional equivalent thereof in accordance with the invention. Suitably therefore the invention may further provide a method of treating a subject who has been diagnosed with low BH4 bioavailability or who has been selected for having a marker of a disorder associated with low BH4 bioavailability or displaying a symptom of a disorder associated with low BH4 bioavailability. Suitably by means of the method of testing described herein.

Suitably the one or more symptoms of a disorder associated with low BH4 bioavailability are defined hereinabove.

Suitably the one or more markers of a disorder associated with low BH4 bioavailability may include any of the following:
(i) A low level of BH4;
(ii) A low ratio of BH4 to BH2;
(iii) A low ratio of BH4 to total biopterins;
(iv) A low level of reduced folates;
(v) A low level of reduced folates relative to oxidised folates;
(vi) Reduced forearm blood flow responses;
(vii) Reduced flow-mediated vasodilation;
(viii) Increased circulating sICAM, P-selectin, von Willebrand factor, or microalbuminuria;
(ix) A low level of a neurotransmitter (such as serotonin, dopamine, or their metabolites); and/or
(x) Increased level of phenylalanine or related metabolites
(xi) A high ratio of sFlt-1 to PIGF
(xii) A disturbed folate metabolism due to e.g. a defect in homocysteine methyltransferase or a deficiency of Vitamin B12
(xiii) A high plasma homocysteine level (optionally due to either vitamin B-12 or folate deficiency)
(xiv) A high methylmalonic acid level (optionally due to vitamin B-12 deficiency)

Suitably by ‘low’, ‘reduced’, ‘high’ or ‘increased’ it is meant relative to the same marker when compared to a reference level measured in a healthy subject.

Suitably testing the subject may comprise the steps of (a) determining the level any of the above markers in any combination in the subject, and (b) comparing to a reference level of the same marker from a healthy subject. Suitably testing the subject may comprise (a) determining if the subject has any symptoms. Suitably this may be in addition or in the alternative to determining the level of any markers.

In one embodiment, testing the subject comprises the steps of (a) determining the BH4 level of the subject; and (b) comparing the determined BH4 level with a reference BH4 level from a healthy subject.

Suitably determining the level of any of the markers in a subject may comprise (i) obtaining a suitable sample from the subject; and ii) measuring the level of one or more of the chosen markers in the sample.

In one embodiment, determining the BH4 level of a subject may comprise (i) obtaining a suitable sample from the subject; and (ii) measuring the level of BH4 in the sample. Therefore, step (a) may suitably comprise determining the BH4 level in a sample from the subject.

A suitable sample may be a tissue sample, a blood sample, a serum sample, a cerebrospinal fluid sample. Suitably the sample is blood. Suitably the sample is from a tissue, such as endothelium, cardiac tissue, liver tissue, brain tissue.

In one embodiment, the BH4 level can be measured in endothelial cells.

In one embodiment, the BH4 level can be measured in microvesicles found within blood that originate from specific cells or tissues within the subject. In one embodiment, the BH4 level can be measured in placental microvesicles found within blood. Suitably, in such an embodiment, the BH4 level can be measured in microvesicles from endothelial cells, cardiac cells, immune cells, liver cells, neuronal cells, or cancer cells.

In one embodiment, the BH4 level can be measured in microvesicles from a subject who is pregnant. Suitably, in placental microvesicles. Suitably, in such an embodiment, the subject is suspected of having a pregnancy-related reduction in BH4 bioavailability. Suitably, in such an embodiment, the subject is suspected of having pre-eclampsia.

In one embodiment, the BH4 level can be measured in a tissue, suitably in cells of a tissue. Suitably, in such an embodiment, the subject is suspected of having a non-pregnancy related reduction in BH4 bioavailability. Suitably, if the BH4 level is measured in cardiac tissue, the subject is suspected of having a cardiac disease associated with low BH4 bioavailability. Suitably, if the BH4 level is measured in brain tissue, the subject is suspected of having a neurological disease associated with low BH4 bioavailability. Suitably, if the BH4 level is measured in liver tissue, the subject is suspected of having a liver disease associated with low BH4 bioavailability.

Suitably, the method may further comprise a step of processing the sample from the subject. Suitably determining the level of one or more markers in a subject may therefore comprise (i) obtaining a suitable sample from the subject; (ii) processing the sample; and (ii) measuring the level of one or more markers in the sample.

Suitably, in one embodiment, processing the sample may comprise isolating placental or other microvesicles from a blood sample. Suitably, in such an embodiment, determining the BH4 level of a subject may therefore comprise (i) obtaining a blood sample from the subject; (ii) isolating placental microvesicles from the blood sample; and (ii) measuring the level of BH4 in the placental or other microvesicles.

Suitably, placental or other microvesicles may be isolated from the blood by any suitable means of separation, such as centrifugation or filtration.

Suitably, measuring the level a chemical marker in the sample comprises performing an immunoassay, HPLC or mass spectroscopy on the sample in accordance with known techniques. For example, measuring the level of BH4, the ratio of BH4 to BH2, the ratio of BH4 to total biopterins, the level of reduced folates, the level of reduced folates relative to oxidised folates, the level of sICAM, P-selectin, von Willebrand factor, or microalbuminuria, the level of neurotransmitters, the ratio of sFlt-1/PIGF, the level of phenylalanine, the level of polymorphism (C677T) in methyltetrahydrofolate reductase, the level of Vitamin B12, the level of plasma homocysteine, and/or the level of methylmalonic acid may be performed using an immunoassay, HPLC or mass spectroscopy. Suitably most of said markers may be measured in a tissue, blood or serum sample.

Suitably measuring the level of blood flow response or vasodilation may be performed by ultrasound.

Suitably a reduced or low BH4 level compared to a healthy subject comprises a reduction in the level of BH4 by at least 20% when compared to the level in a healthy subject. Suitably a reduced BH4 level compared to a healthy subject comprises a reduction in the level of BH4 by 20%, 25%, 30%, 35%, 40%, 45%, 50% when compared to the level in a healthy subject.

In one embodiment, the BH4 level is measured in endothelial cells, therefore suitably a low BH4 level compared to a healthy subject comprises a reduction in the level of endothelial cell BH4 by 20%, 25%, 30%, 35%, 40%, 45%, 50% when compared to the endothelial cell level of BH4 in a healthy subject

Suitably a high sFlt-1/PIGF ratio comprises a ratio of higher than 38. Suitably a high sFlt-1/PIGF ratio may comprise a ratio of higher than 85 in a subject in early pregnancy. Suitably a high sFlt-1/PIGF ratio may comprise a ratio of higher than 110 in a subject in late pregnancy.

In a further aspect of the invention, there may be provided a method of selecting a subject that may benefit from treatment with a reduced folate and optionally BH4, a precursor or functional equivalent thereof, the method comprising determining the reduced folate level of a subject, comparing the reduced folate level to a reference reduced folate level of a healthy subject, and selecting the subject for treatment if the subject exhibits lower reduced folate levels compared to the reference level.

In one embodiment, the method further comprises providing a treatment to the selected subject. In one embodiment, the treatment comprises a reduced folate and optionally BH4, a precursor, or functional equivalent thereof.

Suitably any of the above method steps or features may apply to a method comprising determining the level of one or more of the markers listed above, with suitable modification to determine and compare the level of the one or more markers of the subject. In one example, the ‘BH4 level’ is replaced with the ‘reduced folate level’ and the ‘low BH4 bioavailability’ is replaced with ‘low reduced folate bioavailability‘ or’a reduced folate deficiency’.

Suitably any of the above method steps or features may apply to a method comprising determining the level of one or more of the markers listed above and/or determining if a subject has any of the symptoms listed above.

Additionally, or alternatively, testing a subject for one or more markers of a disorder associated with a low BH4 bioavailability, may comprise testing a subject for impaired activity of one or more enzymes involved in the synthesis of BH4, or impaired activity of one or more biopterin dependent enzymes.

According to a further aspect of the present invention, there is provided a method of selecting a subject that may benefit from treatment with a reduced folate and optionally BH4, a precursor or functional equivalent thereof, the method comprising determining the activity of one or more enzymes in the pterin biosynthetic pathway in a subject, comparing the activity of the or each enzyme to the activity of one or more reference enzymes in a healthy subject, and selecting the subject for treatment if the patient exhibits impaired activity of one or more of the enzymes compared to the reference enzymes.

Suitably the pterin biosynthetic pathway may be the BH4 biosynthetic pathway.

Suitably each reference enzyme is the same enzyme as that which is from the subject undergoing testing, but present in a healthy subject.

Suitably, the method may further comprise determining the BH4 level of a subject and comparing the BH4 level to a reference BH4 level of a healthy subject. Suitably the method may further comprise selecting the subject for treatment if the subject exhibits impaired activity of one or more of the enzymes in the pterin biosynthetic pathway compared to the reference enzyme/s in a healthy subject and/or reduced BH4 levels compared to the reference level in a healthy subject. Suitably impaired activity of one or more of the enzymes will be detected by reduced levels of the product of the enzyme, or reduced substrate consumption by the enzyme, suitably during an enzyme activity assay. Suitably, therefore the method may comprise determining the substrate consumption or the production of product of one or more enzymes in the pterin biosynthetic pathway in a subject. Suitably, therefore the method may comprise determining the production of product of one or more enzymes in the pterin biosynthetic pathway in a subject, using an enzyme activity assay.

The one or more enzymes in the BH4 biosynthetic pathway are defined elsewhere herein. Suitably the method may comprise determining the activity of one or more enzymes selected from: GTP cyclohydrolase I (GTPCH), 6-pyruvoyl-tetrahydropterin synthase (PTPS), sepiapterin reductase (SP), dihydrofolate reductase (DHFR), dihydropteridine reductase (DHPR), pterin-4a-carbinolamine dehydratase (PCD), and endothelial NOS (eNOS).

Methods for determining the activity of enzymes are well known in the art and may include various assays. Suitably such assays comprise incubating the enzyme to be tested with a substrate for a period of time to allow for conversion of the substrate to a product to occur, the consumption of substrate or the production of product may then be measured. The level of product may be measured using HPLC or mass spectroscopy, for example.

Suitably an enzyme activity assay for GTPCH may involve measurement of the production of dihydroneopterin triphosphate from GTP, with a dephosphorylation step that enables quantification of neopterin by fluorescence HPLC. Suitably an enzyme activity assay for DHFR may involve quantification of the production of tetrahydrofolate from dihydrofolate, by HPLC with electrochemical detection.

Suitably in the present invention, production of the enzyme product is measured. Suitably the product being measured is a pterin. Suitably determining the activity of one or more enzymes in the pterin biosynthetic pathway therefore comprises measuring the level of pterin product produced by the or each enzyme in an enzyme activity assay. Suitably the pterin is the relevant pterin produced by the enzyme being tested. Suitably a reduced level of the expected product from the enzyme being tested, compared to a reference enzyme, indicates that the enzyme being tested is impaired. Suitably a reduced level of the pterin product from the enzyme being tested, compared to a reference enzyme, indicates that the enzyme being tested is impaired.

In one embodiment the method may comprise determining the GTP cyclohydrolase I (GTPCH) activity of the subject, and suitably comparing it to a reference GTP cyclohydrolase I (GTPCH) activity of a healthy subject. In one embodiment, the method further comprises selecting the subject for treatment if the subject exhibits impaired GTP cyclohydrolase I (GTPCH) activity. Suitably impaired GTP cyclohydrolase I (GTPCH) activity may be detected by reduced levels of the product 7,8-Dihydroneopterin triphosphate (DHNTP), suitably measured using an enzyme activity assay as described above.

Suitably impaired activity of an enzyme in the pterin biosynthetic pathway comprises a reduction in activity of an enzyme of at least 20% when compared to the activity of the same enzyme in a healthy subject. Suitably impaired activity of an enzyme in the pterin biosynthetic pathway comprises a reduction in activity of an enzyme of 20%, 25%, 30%, 35%, 40%, 45%, 50% when compared to the activity of the same enzyme in a healthy subject.

### Kit

The present invention further provides a kit comprising the two active pharmaceutical ingredients that may be used to form a therapy for prevention or treatment of low BH4 bioavailability as described herein.

The kit comprises at least a reduced folate and optionally BH4, or a precursor or functional equivalent thereof. Suitably the reduced folate and/or BH4, or a precursor or functional equivalent thereof may be stored in appropriate containers. Suitable containers may be, for example, ampoules, bags, bottles, syringes, vials, or blister packaging. Suitably each container contains, for example, one dose or a suitable division of a dose.

Suitably the blister packaging may contain tablets. Suitably each tablet is formulated to contain, for example, one dose or a suitable division of a dose.

Suitably the kit may further comprise instructions for use. Suitably the instructions may inform the user of correct dosage for a subject. Such a kit will suitably have labels or package inserts indicating that the associated active pharmaceutical ingredients are useful for treating a subject suffering from, or predisposed to a disorder associated with low BH4 bioavailability.

Suitably the kit may further comprise one or more excipients, diluents or carriers. Suitably the excipients, diluents or carriers may be for mixing with the reduced folate and/or BH4, or a precursor or functional equivalent thereof. Suitably the instructions may inform the user how to mix the excipients with the reduced folate and/or BH4, or a precursor or functional equivalent thereof.

Suitably the kit may comprise equipment for administering the reduced folate and/or BH4, or a precursor or functional equivalent thereof. For example, the kit may comprise syringes. Suitably the instructions may inform the user how to administer the reduced folate and/or BH4, or a precursor or functional equivalent thereof to a subject.

### In Vitro Uses

The present invention is based on the discovery that a reduced folate can prevent unwanted oxidation of BH4 into the less useful form of BH2. This also has uses in vitro within the laboratory. The thirteenth aspect defines such an in vitro use of the present invention.

Suitably the reduced folate may be used to prevent oxidation of BH4, or a precursor, or functional equivalent thereof. Suitably the reduced folate may be used preserve BH4, or a precursor, or functional equivalent thereof.

Suitably in such embodiments, the reduced folate may be in its natural or unnatural stereoisomeric form.

Suitably the reduced folate may be added to any laboratory process which includes BH4, or a precursor, or functional equivalent thereof. Suitably to any laboratory process which requires BH4 or a precursor, or functional equivalent thereof to remain in a reduced state.

Suitably therefore, reduced folate may be added to stabilise BH4 in biological samples, either during laboratory processes or during storage. Suitably, during laboratory processes, BH4 may be a required substrate or cofactor. Suitable laboratory processes may include assays, such as enzymatic assays. Suitably, such enzymatic assays where BH4 is a required cofactor may include assays involving: tryptophan hydroxylase, phenylalanine hydroxylase, tyrosine hydroxylase, nitric oxide synthase, and ether lipid oxidase.

Suitably the reduced folate may be added to prevent oxidation of BH4, or a precursor, or functional equivalent thereof in storage. Suitably to preserve the BH4, or a precursor, or functional equivalent thereof. Suitably, the invention includes use of a reduced folate as a preservative, to preserve BH4, or a precursor, or functional equivalent thereof. Suitably therefore, the reduced folate may be added to a container containing BH4, or a precursor, or functional equivalent thereof. Suitably therefore, the reduced folate may be added as a preservative to a container containing BH4, or a precursor, or functional equivalent thereof.

In a further aspect of the invention, there is provided a container comprising BH4, or a precursor, or functional equivalent thereof; and a preservative, wherein the preservative is a reduced folate.

Suitably, in either a laboratory process or in a container, the reduced folate is present at a suitable concentration prevent oxidation of BH4 or a precursor, or functional equivalent thereof. Suitably the reduced folate is present in roughly equimolar concentrations compared to BH4. Suitably, therefore, in either a laboratory process or in a container, the reduced folate is present at around 1:1 ratio relative to the BH4 present in the laboratory process or present in the container.

### EXAMPLES

### 1. Methods:

### 1.1 Clinical Cohort

Pregnant women under the care of the Oxford University Hospitals NHS Foundation Trust between 2011 and 2015 were invited to take part in clinical studies, as previously described¹⁻³. Mothers and infants were recruited from normotensive pregnancies, pregnancy induced hypertension and preeclampsia, defined according to ISSHP guidelines. Blood samples were collected at the time of birth. All mothers gave written informed consent, as well as assent for involvement of their children, including permission to access maternal and offspring clinical records. Mothers below the age of 16 years were excluded from the study as were those with chronic cardiovascular conditions prenatally, including pre-existing hypertension¹. Ethical approval was granted by South Central Berkshire Research Ethics Committee ref. 11/SC/0006, clinicaltrials.gov ref. NCT01888770^{2,3}.

### 1.2 HUVEC Isolation, HutMECS Culture and Matrigel Assay

Umbilical cords were collected at birth and human umbilical vein endothelial cells (HUVECs) were isolated and stored in liquid nitrogen according to standard operating procedures within a research tissue bank (Oxford Cardiovascular Tissue Bioresource; ethical approval 09/H0606/68, 07/H0606/148 and 11/SC/0230). For the purpose of the current study, HUVECs were identified from normotensive pregnancies and pregnancies complicated by preeclampsia, matched for maternal age and gestation. HUVECs and human uterine microvascular endothelial cells (HutMECs; Cat C-12295, Promocell) were cultured in EBM2 (endothelial basal medium) with bullet kit as recommended (Cat CC-3162, Lonza). All cell cultures were maintained in humidified 5% CO2 at 37° C. Primary HUVEC and HutMEC cells, obtained between passages 1-3 were and passages 4-6, respectively, were used for all experiments. sEnd.1 endothelial cells were grown in Dulbecco's modified Eagle's medium (Invitrogen) supplemented with glutamine (2 mmol/liter), penicillin (100 units/ml), and streptomycin (0.1 mg/ml).

To assess tube formation ability of endothelial cells, a 96-well plate was evenly coated with 50 μl of growth factor-reduced Matrigel (BD Biosciences, UK). Endothelial cells were plated at a density of 1×10⁴ cells per well. The plate was incubated at 37° C. for 16 hours before photomicroscopy. Each sample was replicated in triplicate and the image of each well was taken at ×4 magnification using a Nikon Eclipse TE2000-U microscope (Nikon Ltd, London, UK). Images obtained from Matrigel assay were adjusted for mean brightness using acquisition software to control the bright field illumination of the microscope (Simple PCI version 6.6.0.0; Hamamatsu Corporation, Sewickley, PA). Images were saved as TIFF files, and tube formation analysed using AngioSys 1.0 (TCS Cell Works, UK). Image threshold was adjusted based on the intensity values of the monochrome image and each image then skeletonized to reduce to one pixel wide. A line was drawn over each tubule and each branch point marked with a dot. The total length of lines was quantified in pixels (then converted to micrometers) and total number of branch points was recorded.

### 1.3 GCH1 Knockdown by RNA Interference

GCH1-specific, ON-TARGETplus SMARTpool siRNA was purchased from Dharmacon Thermo Scientific. 24 h prior to transfection, the cells were seeded into 6-well plates. The cells were then transfected with GCH1-specific siRNA (100 nmol/liter), GAPDH-positive (100 nmol/liter) or nonspecific pooled duplex negative control siRNA (100 nmol/liter). The cells were cultured for 72 h, and gene silencing was detected by analysis of GTPCH protein expression by Western blotting using GTPCH-specific antibodies (a gift from S.Gross, Cornell University New York), in cells using standard protocols.

1.4 Isolation of Placental Extracellular vesicles

Syncytiotrophoblast-derived extracellular vesicles (STBEV) were prepared using a modified dual-lobe placental perfusion system and differential centrifugation as previously described⁴. Briefly, placentae were perfused for 3 h and the maternal side perfusate was collected and immediately centrifuged (Beckman Coulter Avanti J-20XP centrifuge and Beckman Coulter JS-5.3 swing out rotor) twice at 1500×g for 10 min at 4° C. to remove erythrocytes and large cellular debris. The supernatant was centrifuged at 150000 g for 3 hours to collect micro- and nanovesicles. Nanoparticle tracking analysis and flow cytometry were used as previously described to confirm the placental origin and size distribution of particles in the sample⁵. After collection, the STBEVs were diluted in filtered phosphate-buffered salt solution (PBS) (4.9 mg protein/ml), and frozen (−80° C.) until further use in vascular experiments.

### 1.5 Maternal Blood Sample Analysis

Plasma circulating pro-angiogenic and anti-angiogenic factors were quantified with commercial enzyme-linked immunosorbent assays (ELISAs). All samples, standards, and controls were plated in duplicate. Optical density of each well was measured at 450 nm using a FLUOstar Omega microplate reader (BMG Labtech, KBioScience, USA). Data was analyzed using Omega Data Analysis software. Duplicate readings for each standard, control, and sample were averaged, and the average zero standard optical density was subtracted. Standard curves were created by generating a four-parameter logistic curve-fit. The coefficients of variation for sFlt-1 was 4.5% with a SD of 1.9%, and for sEng it was 4.1% with a SD of 1.6%.

### 1.6 Generation of Endothelial Cell-Targeted Gch1 Knockout Mice

We generated a novel mouse model of endothelial cell-specific BH4 deficiency, the Gch1^{fl/fl}Tie2cre mouse. Exons 2 and 3 of Gch1, encoding for the active site of GTPCH, were flanked by loxP sites in a targeting construct that was used to produce Gch1^{fl/fl} mice after homologous recombination in embryonic stem cells. These mice were crossed with Tie2cre transgenic mice to produce Gch1^{fl/fl}Tie2cre mice where Gch1 is deleted specifically in endothelial cells, generating an endothelial cell BH4-deficient mouse⁶. Mice were housed in ventilated cages with a 12-hour light/dark cycle and controlled temperature (20-22° C.), and fed normal chow and water ad libitum. Female Gch1^{fl/fl}Tie2cre mice and their Gch1^{fl/fl} littermates (thereafter referred to as wild-type) were used for all experiments at 10 to 16 weeks. All studies were conducted in accordance with the UK Home Office Animals (Scientific Procedures) Act 1986 (HMSO, London, United Kingdom). Mice were genotyped by polymerase chain reactions using DNA prepared from ear biopsies. For Gch1^{fl/fl} genotyping, PCR was performed using the following primers: Gch1 fl/fl-Fw 5′-GTC CTT GGT CTC AGT AAA CTT GCC AGG-3′, Gch1 fl/fl-Rv 5′-GCC CAG CCA AGG ATA GAT GCA G-3′. The Gch1 floxed allele showed a 1030 bp. For Tie2cre genotyping, PCR was performed using the following primers: Tie2cre Fw 5′-GCA TAA CCA GTG AAA CAG CAT TGC TG-3′. Tie2cre Rv 5′-GGA CAT GTT CAG GGA TCG CCA GGC G-3′. The Tie2cre allele amplified as 280 bp fragment.

### 1.7 Timed Mating

Pregnancy was achieved by mating either virgin female Gch1^{fl/fl}Tie2cre or Gch1^{fl/fl} (wildtype) females (aged between 10 to 16 weeks old) with a Gch1^{fl/fl} male. To evaluate the gestation day, vaginal plugs were checked for the following morning, taken as the 0.5 day of gestation (E0.5). Body weights of plugged Gch1^{fl/fl}Tie2cre and wild-type mice were determined throughout gestation (E0, E2.5, E5.5, E7.5, E10.5, E12.5, E15.5, E16.5, E17.5 and E18.5). Urine samples from non-pregnant and pregnant (at E18.5) Gch1^{fl/fl}Tie2cre and wild-type females were collected and stored at −80° C. for biochemistry analysis. Unless otherwise stated, all tissues were harvested and collected for experiments at either preconception (before timed mating) or E18.5 day of gestation (late gestation, two days prior to normal term delivery).

### 1.8 BH4 and Biopterin Measurements

BH4 and oxidised biopterins (BH2 and biopterin) in plasma and uterine arteries were determined by high-performance liquid chromatography (HPLC) followed by electrochemical and fluorescent detection, respectively, following established protocols^{7,8} Briefly, samples were freeze-thawed in ice-cold resuspension buffer (50 mM phosphate buffered saline, 1 mM dithioerythriol, 1 mM EDTA, pH 7.4). After centrifugation at 13,200 rpm for 10 min at 4° C., supernatant was removed and ice-cold acid precipitation buffer (1 M phosphoric acid, 2 M trichloroacetic acid, 1 mM dithioerythritol) was added. Following centrifugation at 13,200 rpm for 10 min at 4° C., the supernatant was removed and injected onto the HPLC system. Quantification of BH4 and oxidised biopterins was obtained by comparison with external standards and normalised to protein concentration, determined by the BCA protein assay.

### 1.9 Vascular Function Studies

Vasomotor function in uterine arteries and aortas from both non-pregnant and pregnant (E18.5) Gch1 fl/flTie2cre and wild-type littermates was examined using isometric tension studies in a wire myograph (MultiMyogrph 610M, Danish Myo Technology, Denmark)⁶. Briefly, mice were culled by overdose of inhaled isoflurane and vascular rings were isolated from the uterine horns or thoracic aorta. The 2-mm rings of uterine arteries (main loop) or aortic rings were then mounted in a wire myograph containing 5 ml of ice-cold KrebsHenseleit buffer (KHB [in mmol/l]: NaCl 120, KCl 4.7, MgSO4 1.2, KH2PO4 1.2, CaCl2 2.5, NaHCO₃25, glucose 5.5) at 37° C., gassed with 95% O2/5% C02. After allowing vessels to equilibrate for 30 minutes, the optimal tension was set (equivalent to 100 mmHg). Concentration-response contraction curves were established using cumulative half-log concentrations of U46619 (thromboxane A2 receptor agonist) and phenylephrine respectively. Vessels were washed three times with fresh KHB, equilibrated for 20 minutes, and then precontracted to approximately 80-90% of maximal tension with U46619 for uterine arteries or with phenylephrine for aortas. Acetylcholine was used to stimulate endothelium dependent vasodilatations in increasing cumulative concentrations. Responses were expressed as a percentage of the pre-contracted tension. Finally, the NO donor sodium nitroprusside (SNP) was used to test endothelium-independent smooth muscle relaxation in the presence of L-NAME. All pharmacological drugs were pre-incubated at least 20 min before the dose-response curves were determined. L-NAME was used at 100 μM, apamin at 50 nM, charybdotoxin at 100 nM, and indomethacin at 10 μM. All drugs used were purchased from Sigma Chemical Company.

### 1.10 Blood Pressure Measurement by Tail-Cuff Plethysmography

Systolic blood pressure and heart rate was determined using a computerized tail-cuff system (Visitech, USA) in conscious mice⁶. Experiments were performed between the hours of 8 and 12 am. The animal tails were passed through a cylindrical latex tail-cuff and taped down to reduce movement. Twenty readings were taken per mouse of which the first 5 readings were discarded. The remaining 15 readings were used to calculate the mean systolic blood pressure and heart rate in each mouse.

### 1.11 Blood Pressure Measurement by Implantable Telemetry

Non-pregnant female Gch1fl/flTie2cre and Gch1fl/fl (wild-type) mice (8-10-week-old) underwent thoracic aortic implantation of telemeters (PAC10 radiotelemeters; DSI, Transoma Medical Inc.) as described previously. Briefly, telemeter catheters were implanted in the left carotid artery with the body of the telemeter placed in a subcutaneous pocket equidistant from the fore and hind paw. The wound was then closed with 4.0 Vicryl. Post-operatively, mice were held in a recovery chamber at 37° until mobile and subsequently moved to a recovery cabinet at 28° for a further 4 h. After 14 days of recovery in home cages (placed on top of telemetry receivers), telemeters were magnetically activated, and baseline mean arterial blood pressure (MAP) was recorded continuously for 5 days (with sampling every 1 minutes for 10-second intervals). Pregnancy was achieved by mating either female Gch1^{fl/fl}Tie2cre or Gch1^{fl/fl} (wild-type) females with a Gch1^{fl/fl} male. To evaluate the gestation day, vaginal plugs were checked for the following morning, taken as the 0.5 day of gestation (E0.5). MAP was recorded continuously throughout the pregnancy until E18.5 day of gestation.

1.12 Analysis of NO Synthesis by eNOS

NO synthesis by eNOS was assessed by conversion of ¹⁴C L-arginine to citrulline, in the presence and absence of N-monomethyl-L-arginine (1 mM, Sigma), as described previously⁹. Briefly, HUVES were incubated for 4 hours at 37° C. in 200 μl Krebs-HEPES buffer containing ¹⁴C L-arginine (2 μl of 50 μCi/mL). Samples were run on a SCX 300 cation exchange HPLC column (Sigma) with online scintillation detection. Background signals were corrected from samples with ¹⁴C L-arginine alone without cells.

### 1.13 Micro CT Imaging

Placentas were imaged using a SkyScan 1172 micro-CT (Bruker). The placentas were mounted in 1.5% agarose in a sealed sample holder. X-ray images were generated at a voltage of 45kv and a current of 218 μA, with no filter applied. Scanning resolution was set at 2.5 μM per pixel. A virtual image stack generated using NRecon software (Bruker). The image stack was downsized to a resolution of 10 μM per pixel. 3D reconstructions were generated using AMIRA software (version 5.5.0).

### 1.14 Histology and Immunostaining

Placentas and uterine arteries from wild-type and Gch1^{fl/fl}Tie2cre mice at E18.5 day of gestation were harvested following perfusion fixation at 100 mmHg. Paraffin-embedded placentas and uterine arteries were stained with H&E and immunohistochemistry for a smooth muscle actin (Sigma), according to the manufacturer's instructions.

### 1.15 Statistical Analysis

Data are presented as mean±SEM. Normality was tested using D'Agostino and Pearson omnibus normality test. Groups were compared using the Mann-Whitney U test for nonparametric data or an un-paired Student's t-test for parametric data. When comparing multiple groups data were analysed by analysis of variance (ANOVA) with Newman-Keuls post-test for parametric data or Kruskal-Wallis test with Dunns post-test for non-parametric data. When more than two independent variables were present a two-way ANOVA with Tukey's multiple comparisons test was used. When within subject repeated measurements were present a repeated measures (RM) ANOVA was used. A value of P<0.05 was considered statistically significant.

### 1.16 Quantification of Superoxide Production by HPLC

Measurement of 2-hydroxyethidium formation by HPLC was used to quantify superoxide production, as previously described. Briefly, the cells were washed three times in PBS and incubated in Krebs-Hepes buffer in the presence or absence of I-NAME (100 μm). After 30 min, dihydroethidium (25 μm) was added, and the cells were then incubated for an additional 20 min at 37° C. The cells were then harvested by scraping, centrifuged, and lysed in ice-cold methanol. Hydrochloric acid (100 mm) was added (1:1 v/v) prior to loading into the autosampler for analysis. All of the samples were stored in darkened tubes and protected from light at all times. Separation of ethidium, oxyethidium, and dihydroethidium was performed using a gradient HPLC system (Jasco) with an ODS3 reverse phase column (250 mm, 4.5 mm; Hichrom), and quantified using a fluorescence detector set at 510 nm (excitation) and 595 nm (emission). A linear gradient was applied from Mobile phase A (0.1% TFA (v/v)) to Mobile phase B (0.1% TFA (v/v) in acetonitrile) over 23 min (30% acetonitrile to 50% acetonitrile).

### 1.17 Immunoprecipitation and Western Blotting

Following exposure of cells with the appropriate treatment, the cells were suspended in radioimmune precipitation assay lysis buffer (20 mm Tris-HCl, 150 mm NaCl, 20 mm N-ethylmaleimide, 1 mm Na2EDTA, 1 mm EGTA, 1% Triton (v/v), 0.1% SDS (w/v), 0.1 sodium deoxycholate, pH 7.4), including a mixture of protease inhibitors (Roche Applied Science), and subjected to three freeze-thaw cycles in liquid nitrogen. Western blotting was carried out using standard techniques with either of rabbit anti-mouse GTPCH antibody (Gifted by Prof Steve Gross), anti-eNOS (BD Transduction Laboratories), and anti-GAPDH (Sigma) antibodies.

### 2. Results

2.1 Pre-eclampsia or Hypertension in Pregnancy is Associated with Reduced Endothelial Cell GTPCH and BH4 Levels, Impaired NOS Activity and Impaired Endothelial Tube Formation

To investigate whether endothelial GCH1 and BH4 are altered in pre-eclampsia, or in pregnancies complicated by hypertension, we measured BH4 levels in primary human umbilical vein endothelial cells (HUVECs) and plasma obtained at birth from women who had pregnancies complicated by pre-eclampsia (PE) and/or hypertension, in comparison with mothers with normotensive pregnancies. The clinical characteristics of the study patients are shown in Table 1:

TABLE 1
Characteristics of Cohort (HUVECs)

| | | |
|---|---|---|
| | Normotensive | Hypertensive |
| Parameters | (n = 14) | (n = 12) |

Maternal

| | | |
|---|---|---|
| Maternal age, years | 33.6 ± 2.9 | 35.4 ± 3.8 |
| BMI at booking, kg/m² | 23.4 ± 2.9 | 30.1 ± 8.2* |
| Smokers, n (%) | 2(14.3) | 4(40) |
| LFT, n (%) | 0(0) | 3(30) |
| Booking sBP, mmHg | 107.1 ± 11.4 | 120.6 ± 12.5 |
| | (86-126) | (90-130)* |
| Booking dBP, mmHg | 64.4 ± 8.6 | 74.9 ± 8.4 |
| | (50-83) | (60-84)* |
| Late gestation sBP, mmHg | 108.6 ± 9.1 | 122.1 ± 16.6 |
| | (90-120) | (102-153)* |
| Late gestation dBP, mmHg | 63.4 ± 8.5 | 81.9 ± 12.7 |
| | (50-81) | (55-93)*** |
| Highest sBP, mmHg | 119.6 ± 13.7 | 157.6 ± 26.0 |
| | (98-148) | (145-200)*** |
| Highest dBP, mmHg | 72.9 ± 8.5 | 97.5 ± 11.2 |
| | (60-87) | (88-135)*** |

Fetal

| | | |
|---|---|---|
| Gestational age, weeks | 39.5 ± 2.1 | 36.7 ± 2.4** |
| Males, n (%) | 8(57.1) | 6(54.5) |
| Birthweight, grams | 3390.4 ± 731.8 | 2655.1 ± 802.3* |
| Birthweight z-score | 0.3 ± 0.9 | −0.6 ± 1.0* |
| Head circumference, mm | 173.8 ± 10.2 | 173.9 ± 10.4 |
| Abdominal circumference, mm | 141.8 ± 41.5 | 152.2 ± 16.3 |
| Femur length, mm | 31.9 ± 2.3 | 31.3 ± 3.2 |

Values as Mean ± Standard Deviation unless stated otherwise.
sBP systolic blood pressure; dBP diastolic blood pressure; LFT: liver function test.
Statistically significant p-values are asterisked.
*p < 0.05;
**p < 0.01;
***p < 0.001

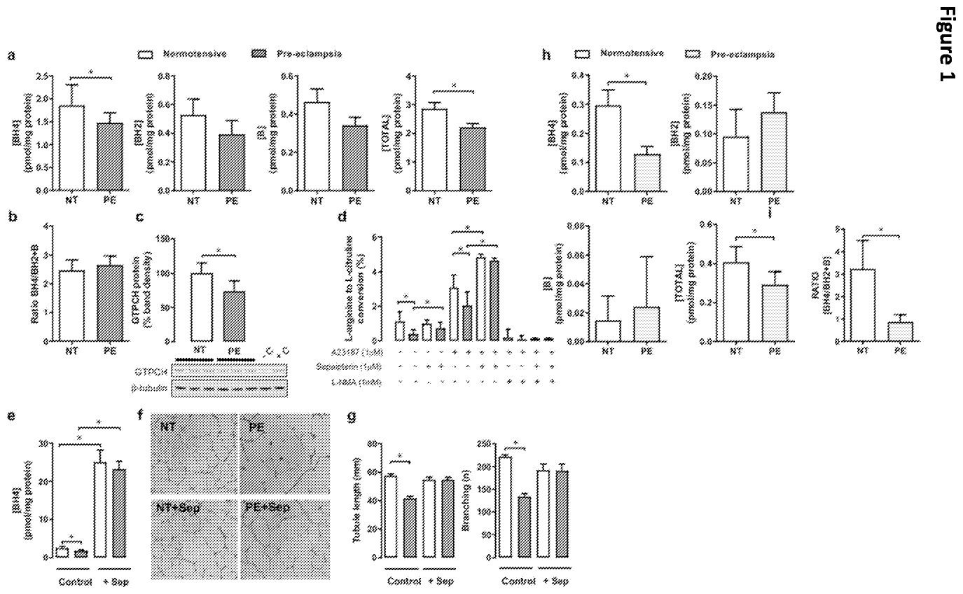

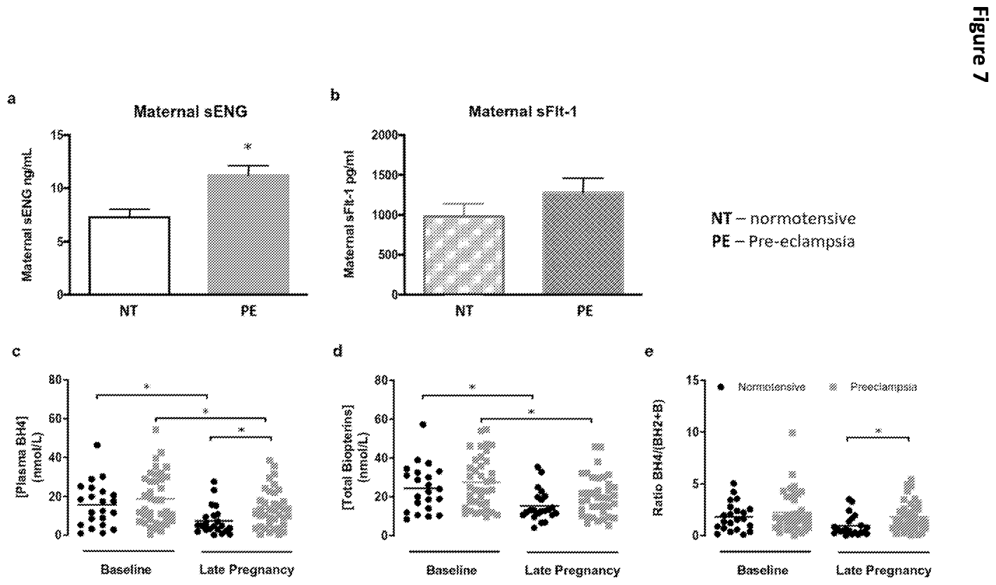

Mothers with preeclampsia and/or hypertension had higher blood pressure, and elevated levels of both soluble endoglin and sFlt1 at 5 days post-partum. We found that the levels of BH4, GTPCH protein and eNOS activity were significantly decreased in HUVECs from pre-eclamptic/hypertensive pregnancies compared with endothelial cells from normotensive pregnancies (FIG. 1a-d). Furthermore, endothelial cell tube formation, a marker of endothelial cell growth, was reduced in HUVECs from hypertensive/pre-eclamptic pregnancies (FIG. 1e-g). To test the dependence of these endothelial cell abnormalities on BH4, incubation with the BH4 precursor, sepiapterin, normalized both BH4 levels and NOS activity in HUVECs from hypertensive/pre-eclamptic pregnancies, such that BH4 levels and NOS activity were no longer different between the groups (FIGS. 1d and e). Furthermore, sepiapterin restored tube formation in HUVECs from hypertensive/pre-eclamptic pregnancies (FIG. 1e-g). In contrast to the reduced levels of BH4 in endothelial cells from hypertensive/pre-eclamptic pregnancies, circulating plasma BH4 levels and total biopterins were significantly higher in mothers with hypertension/pre-eclampsia compared with controls (FIG. 7c-e), and associated with a reduced BH4/BH2 ratio, indicating greater systemic BH4 oxidation. Furthermore, plasma BH4 levels in both normal and hypertensive/pre-eclamptic women were significantly decreased in late pregnancy compared with early pregnancy (FIG. 7). To further investigate the relevance of BH4 in the human placental circulation, we investigated the levels of BH4 in placental extracellular vesicles isolated from perfused placentas obtained from women with or without hypertensive/pre-eclampsia, a model system previously demonstrated to reflect alterations in key aspects of placental vascular function, including the levels of eNOS. We found that BH4 content in placental extracellular vesicles from perfusion of placentas from hypertensive/pre-eclamptic pregnancies was significantly lower than those in placental extracellular vesicles from healthy pregnancies (FIG. 1H).

### 2.2 Knockdown of Gch1 Reduces GTPCH Protein and BH4 Levels and Impairs Endothelial Tube Formation in Endothelial Cells

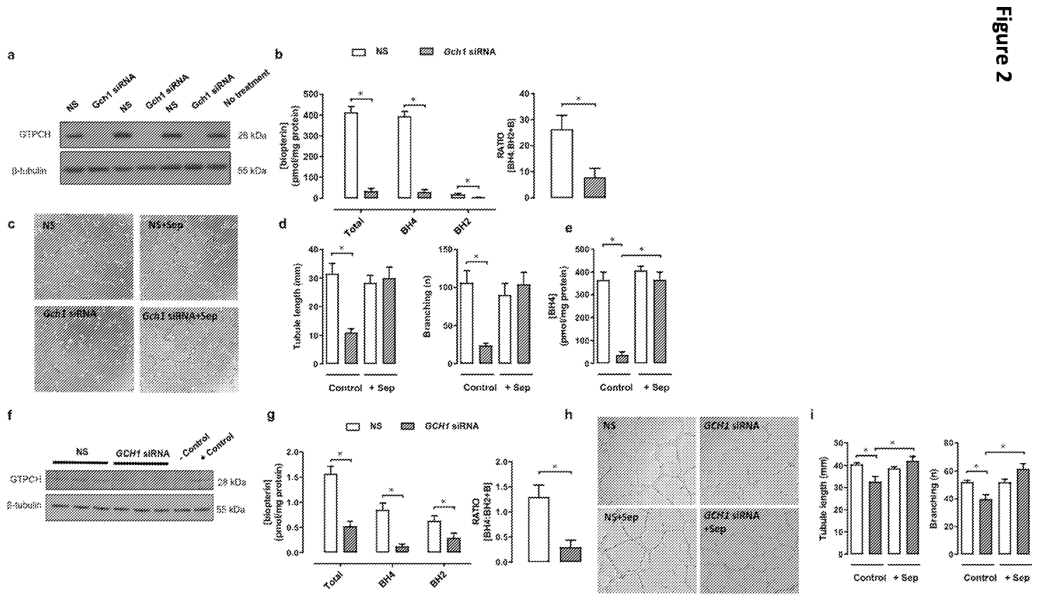

We next tested the causal role of Gch1 and BH4 in endothelial cell tube formation using siRNA knockdown of Gch1 in the mouse sEND.1 endothelial cell line, that have been widely studied as a model system for eNOS regulation by Gch1 and BH4. We found that Gch1-specific siRNA substantially decreased GTPCH protein levels (FIG. 2a) with a corresponding ˜90% reduction in intracellular BH4 levels, compared with non-specific siRNA controls (FIG. 2b). The ratio of BH4 relative to oxidized biopterin species (BH4:BH2+B) was also significantly reduced in cells treated with Gch1-specific siRNA (FIG. 2b). Consistent with the earlier findings in HUVECs, Gch1 knockdown impaired endothelial cell tube formation (FIG. 2c-e), whereas incubation with sepiapterin, that leads to intracellular BH4 synthesis via the pterin salvage pathway, increased BH4 levels and fully restored tube formation (FIG. 2c-e). Thus, Gch1 is required for normal BH4 biosynthesis, eNOS activity and tube formation in cultured endothelial cells, supporting the notion that the reduction in endothelial cell GTPCH and BH4 observed in endothelial cells from preeclamptic pregnancies could play a causal role in the pathogenesis of pre-eclampsia. To further test the relevance of these observations to human maternal endothelial cells, we also knocked down GCH1 in primary human uterine microvascular endothelial cells (HUtMECs). GCH1 knockdown substantially reduced GTPCH protein expression and BH4 levels in HUtMECs (FIGS. 2f and g) and resulted in a significant reduction in endothelial cell growth in tube formation assays (FIGS. 2h and i). Taken together, these observations indicate that endothelial cell GCH1 and BH4 regulate endothelial cell growth and tube formation, that reduced endothelial cell BH4 is a feature of pre-eclampsia, and is associated with loss of endothelial cell NOS activity.

### 2.3 Endothelial Cell-Specific Gch1 Deletion Causes Pregnancy-Induced Hypertension and Fetal Growth Restriction in Female Gch1^{fl/fl}Tie2cre Mice

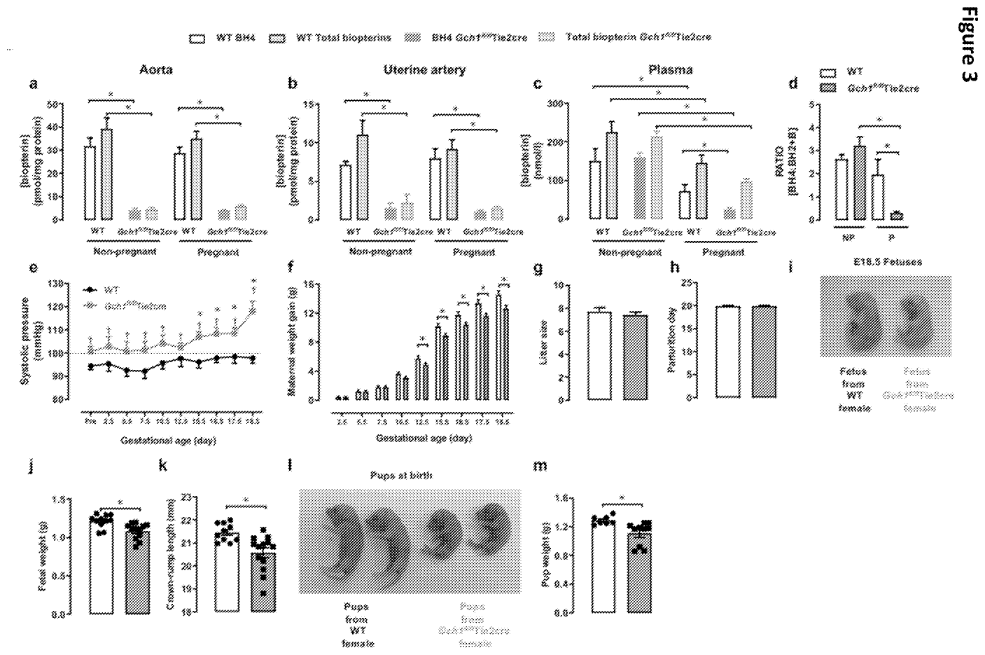

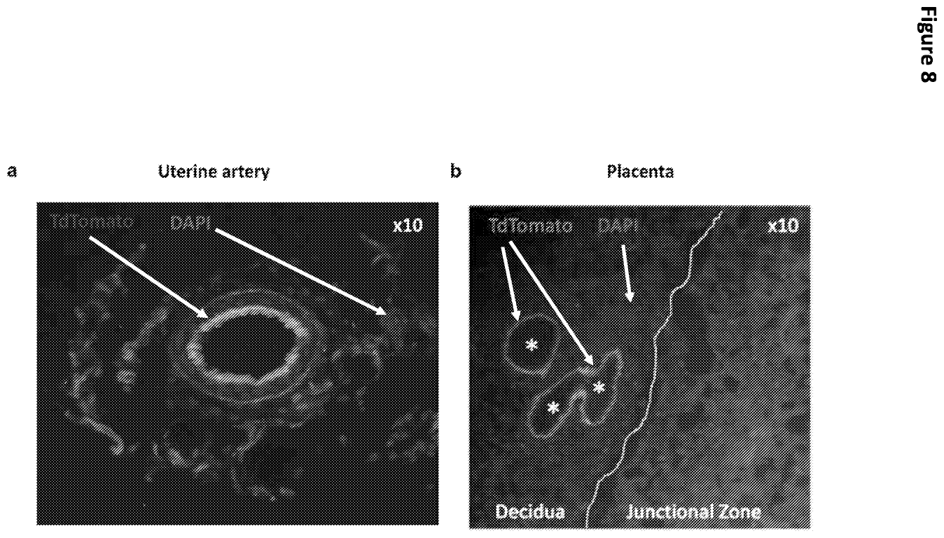

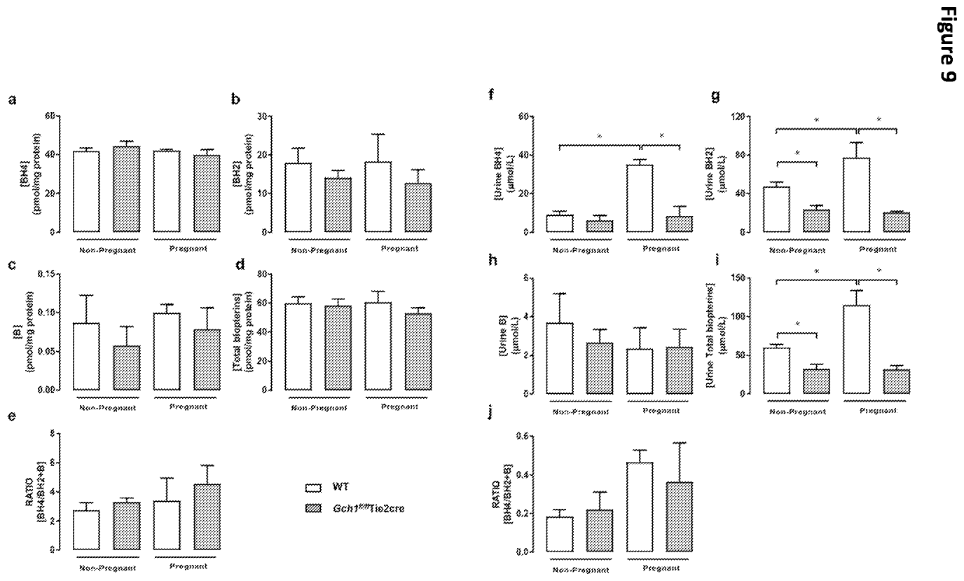

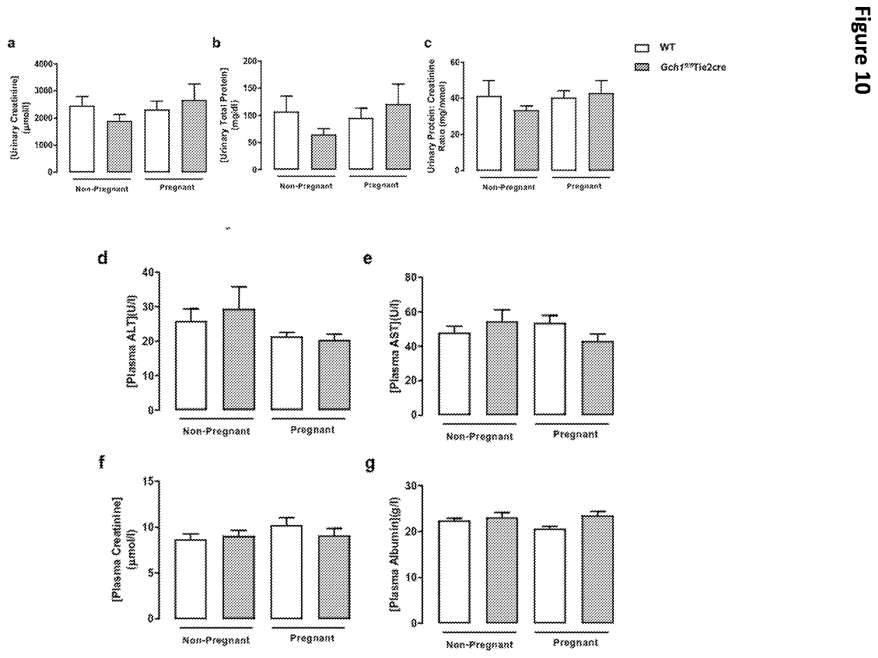

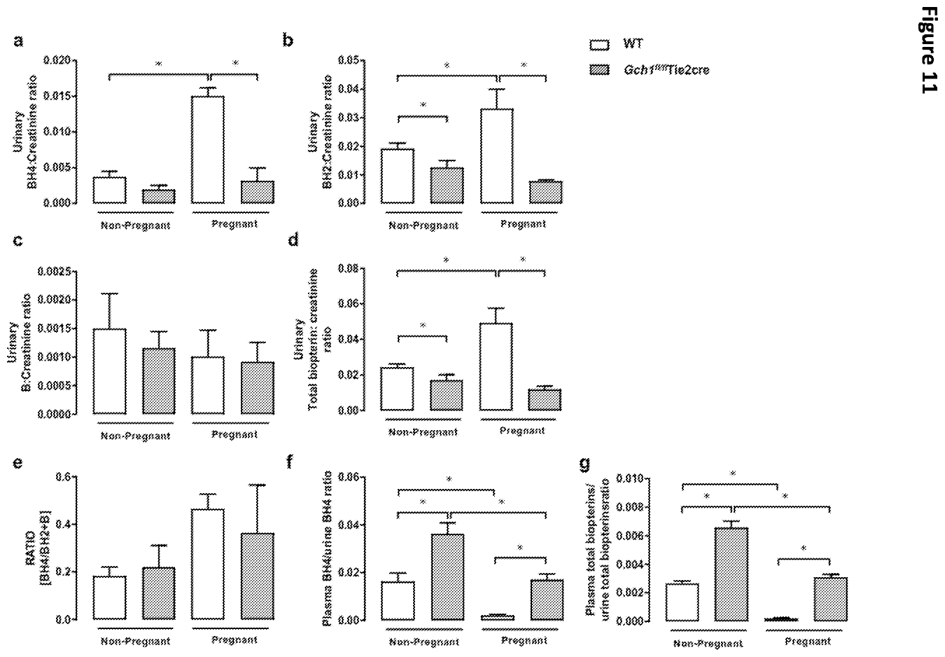

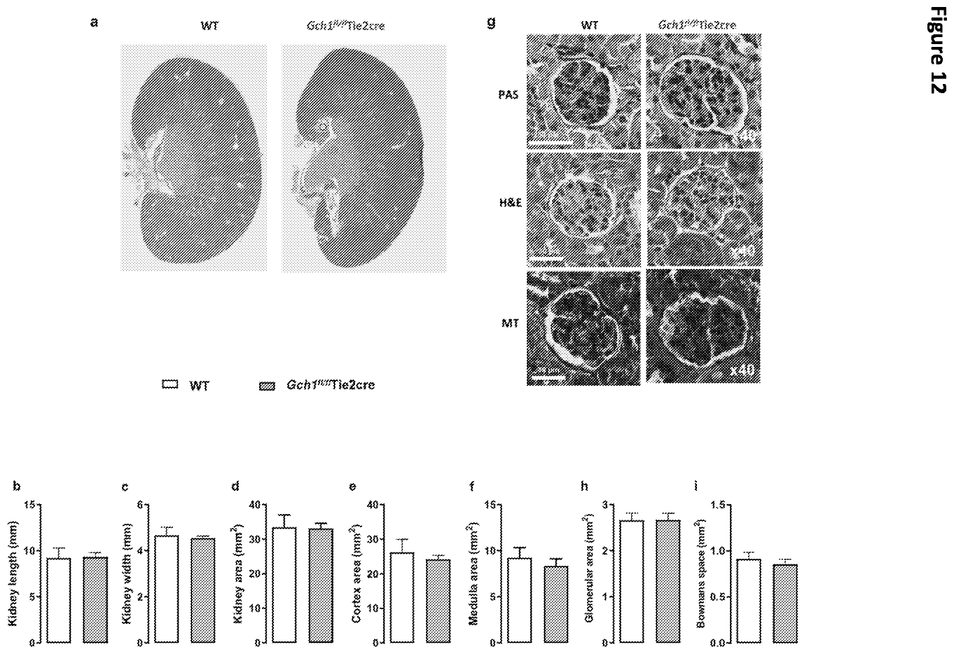

To investigate the specific role of maternal endothelial cell BH4 in uteroplacental vascular adaptation and blood pressure regulation during pregnancy, we next investigated the response to pregnancy in female mice with endothelial cell-specific deletion of Gch1, encoding GTP cyclohydrolase 1. Endothelial cell-specific excision of the floxed allele was confirmed in uterine arteries and in the spiral arteries of the placental decidua, but not in other decidual cells, using fluorescence imaging of tissue sections from Tie2cre mice crossed with tdTomato reporter mice, at day 18.5 of pregnancy (FIG. 8). In non-pregnant mice, BH4 and total biopterins levels in aortas and uterine arteries from Gch1^{fl/fl}Tie2cre mice were significantly lower compared to that of wild-type mice (FIGS. 3a and b), whereas plasma levels of BH4 were not different between genotypes (FIG. 3c), indicating that endothelial cell BH4 synthesis is not a major contributor to circulating biopterin levels in healthy non-pregnant female mice. In pregnant mice, BH4 and total biopterins levels in aortas and uterine arteries were comparable to non-pregnant mice from the same genotype (FIGS. 3a and b). However, plasma levels of BH4 and total biopterins were significantly reduced in pregnant mice, both in wild-type and to a greater extent in Gch1^{fl/fl}Tie2cre mice (FIG. 3c). Furthermore, the BH4:BH2+B (total biopterins) ratio in plasma was significantly reduced in pregnant Gch1fl/flTie2cre mice compared to non-pregnant Gch1^{fl/fl}Tie2cre mice or pregnant wild-type mice (FIG. 3d), indicating that endothelial cell specific BH4 deficiency in pregnancy leads to further reduction in BH4 due to oxidation, forming BH2 and B. The reduction in plasma biopterins in pregnancy was not associated with any difference in liver biopterins, (considered to be the principal source of circulating biopterins), but was associated with a striking increase in urinary biopterins, suggesting that renal excretion of biopterins is increased in pregnancy (FIGS. 9 and 10). However, the pregnancy-associated increase in urinary biopterins was not greater in Gch1^{fl/fl}Tie2cre mice, nor were there any changes in plasma creatinine or in renal histology in Gch1^{fl/fl}Tie2cre mice, indicating that endothelial cell Gch1 and BH4 deletion do not exert effects through changes in renal function (FIGS. 11 and 12).

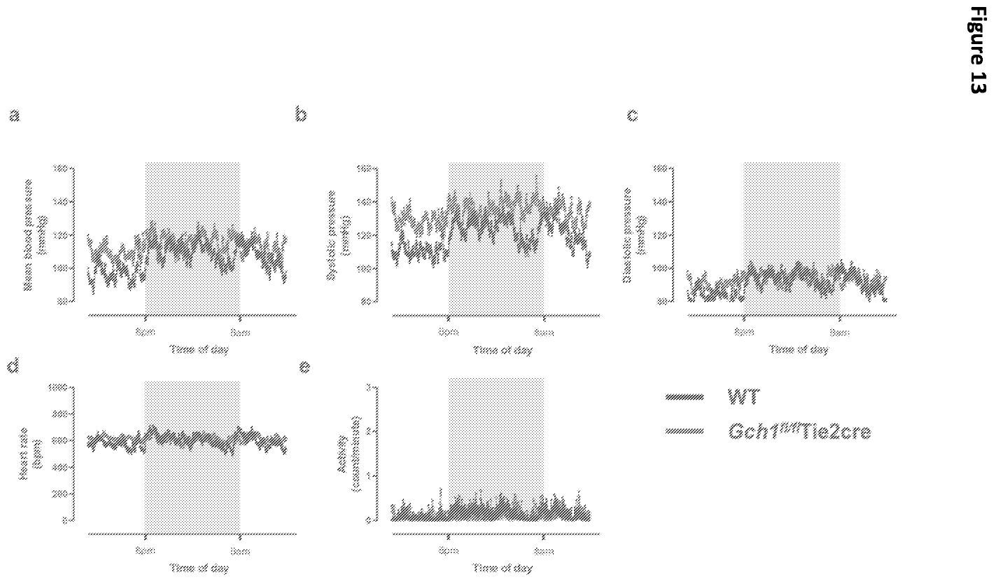

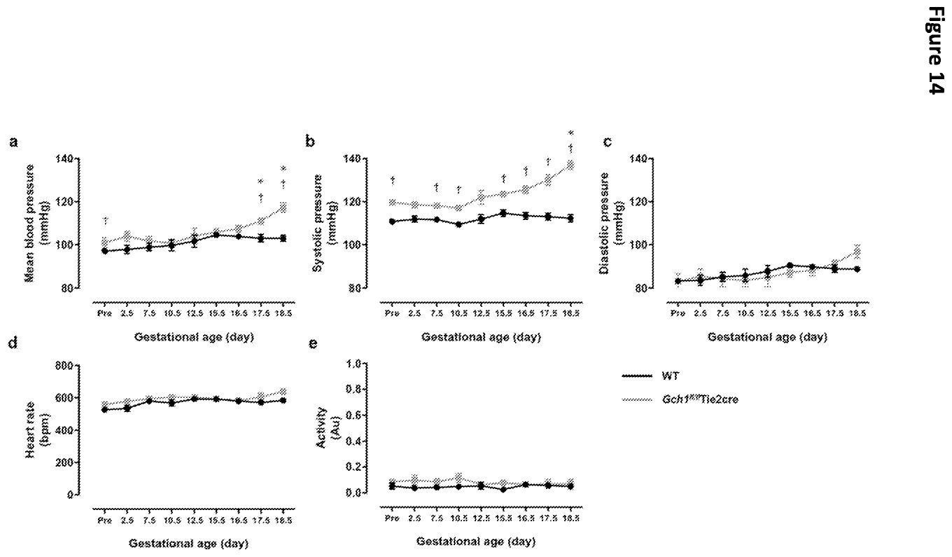

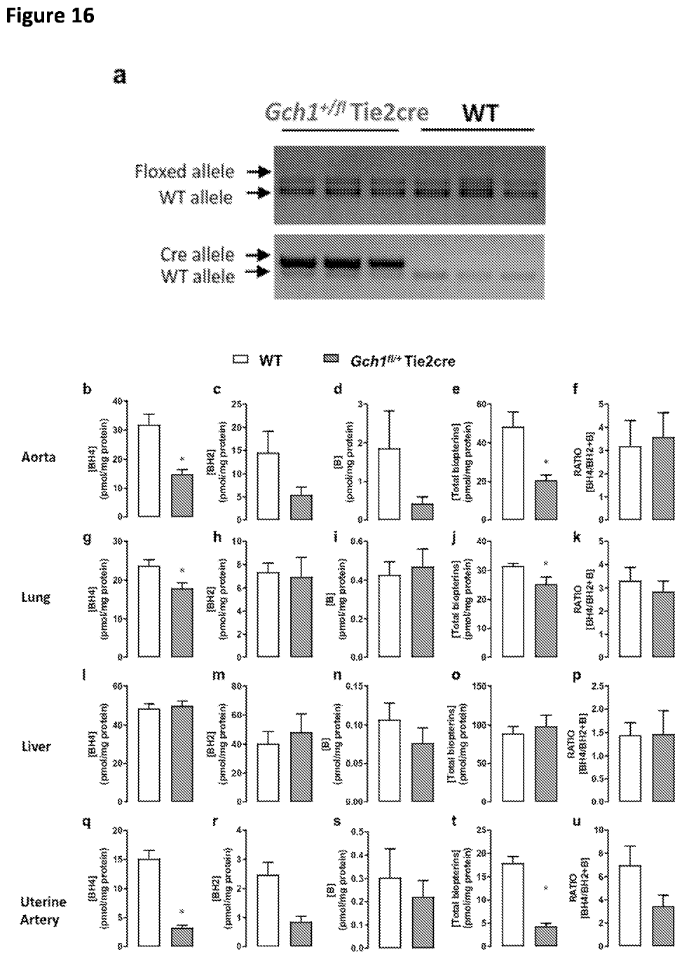

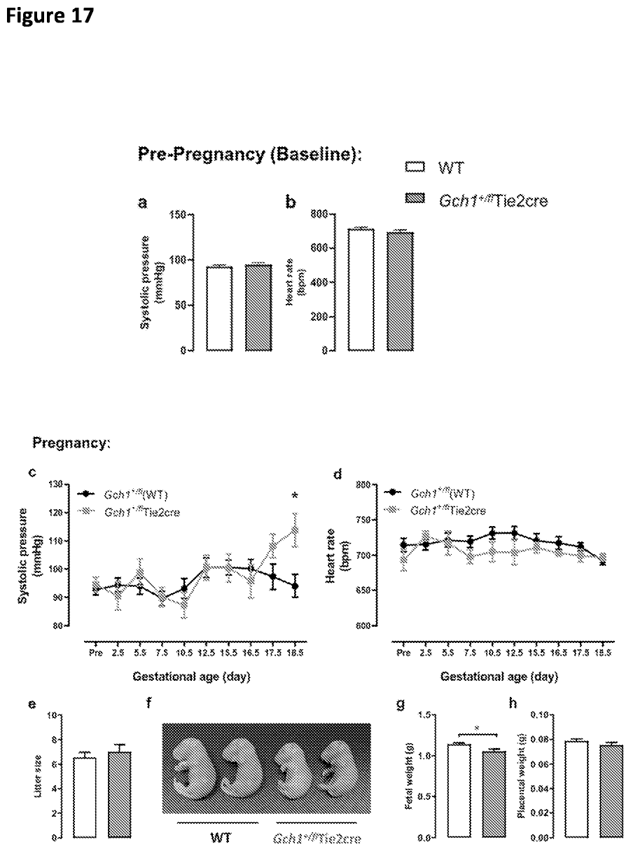

We next determined the requirement for maternal endothelial cell BH4 biosynthesis in blood pressure regulation during pregnancy. We first used tail-cuff plethysmography in pregnant mice. As previously reported (17), systolic blood pressure was slightly higher (˜5 mmHg) in female non-pregnant Gch1^{fl/fl}Tie2cre mice, compared to non-pregnant wild-type littermates (FIG. 3e). However, by day E15.5 of gestation, systolic blood pressure was significantly increased above basal levels and further elevated at E18.5 day of gestation in pregnant Gch1^{fl/fl}Tie2cre mice compared to that of non-pregnant Gch1^{fl/fl}Tie2cre mice (FIG. 3e). In contrast, no significant changes in blood pressure were seen in wild-type mice during pregnancy (FIG. 3e). We further evaluated blood pressure changes using implantable telemeters, implanted before pregnancy. We observed that the progressive increase in blood pressure during pregnancy in Gch1^{fl/fl}Tie2cre mice was driven principally by increased systolic blood pressure, whereas diastolic blood pressure in Gch1^{fl/fl}Tie2cre mice was only significantly increased at the end of pregnancy (FIGS. 13 and 14). To address the potential effect of the small increase in baseline blood pressure in Gch1^{fl/fl}Tie2cre mice, we first analysed additional cohorts of Gch1^{fl/fl}Tie2cre and wild type mice that were matched for baseline blood pressure. In Gch1^{fl/fl}Tie2cre mice with pre-pregnancy blood pressures that were identical to a paired cohort of wild-type mice, the increase in blood pressure during pregnancy was significantly increased (23+/−5 mmHg), whereas the cohort of wild-type demonstrated no increase in blood pressure during pregnancy (FIG. 14). Next, we generated cohorts of mice with endothelial cell-specific deletion of only one Gch1 allele, i.e. Gch1^{fl/+}Tie2cre mice, in order to investigate whether loss of a single Gch1 would be sufficient to cause endothelial cell BH4 deficiency, and if so, the effect on blood pressure. We found that BH4 levels in aorta and lung of Gch1^{fl/+}Tie2cre mice were reduced by approximately 50% compared with wild type mice, and less than the ˜80% reduction in BH4 observed in Gch1^{fl/fl}Tie2cre mice (FIG. 16). Levels of BH4 in liver were unchanged in Gch1^{fl/fl}Tie2cre mice. In pregnant Gch1^{fl/+}Tie2cre mice, BH4 levels were reduced by ˜80% in uterine arteries (FIG. 16). Blood pressure measurements in Gch1^{fl/+}Tie2cre mice revealed no difference in baseline (pre-pregnancy) blood pressure, but a significant increase in blood pressure during late pregnancy was still observed in Gch1^{fl/+}Tie2cre mice, despite the normal baseline blood pressure and reduced level of endothelial cell BH4 deficiency (FIG. 17).

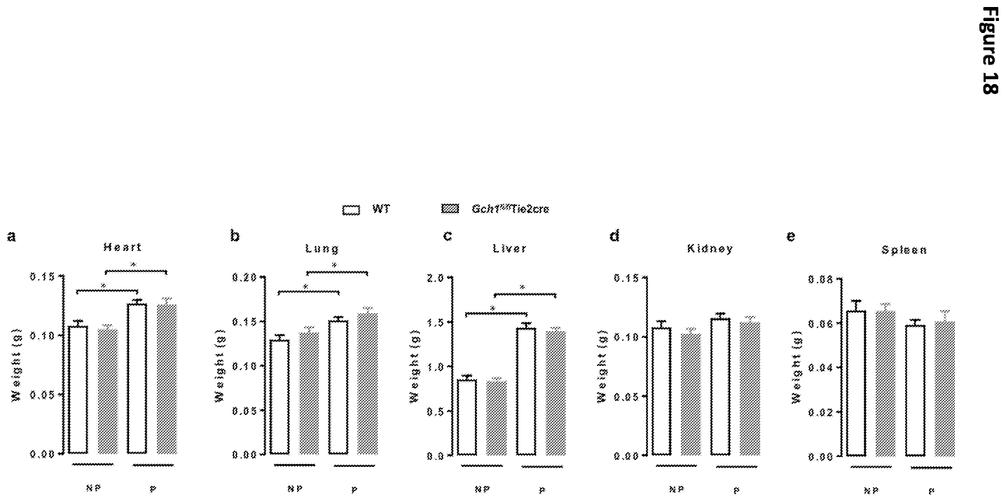

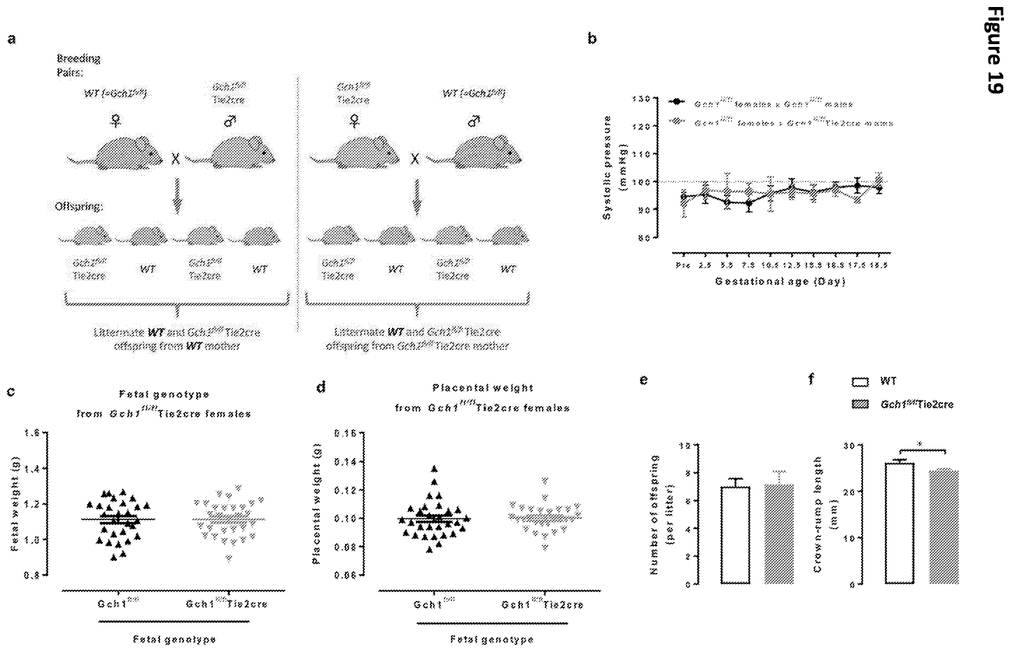

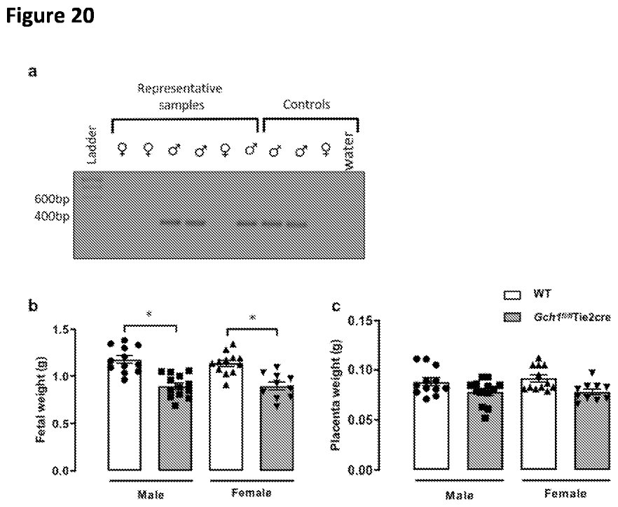

These data indicate that the hypertensive response to pregnancy induced by endothelial cell BH4 deficiency is dose-dependent, but not dependent on the small increase in baseline blood pressure in Gch1^{fl/+}Tie2cre mice. To determine the importance of maternal endothelial cell BH4 on placental and fetal development, the weight gain of Gch1^{fl/fl}Tie2cre and wild-type mice were determined at preconception and throughout pregnancy. Preconception body weights were similar between Gch1^{fl/fl}Tie2cre mice and wild-type littermates. However, maternal weight gain from E12.5 day of pregnancy onwards was significantly reduced in Gch1^{fl/fl}Tie2cre mice compared to wild-type mice littermates (FIG. 3f), that was not caused by any difference in the organ weights of heart, liver, lung, kidney or spleen (FIG. 18). There were no significant differences in the number of pups per litter between Gch1^{fl/fl}Tie2cre and wild-type mice at E18.5 day of gestation or at birth (FIG. 3g). The onset of parturition was comparable between Gch1^{fl/fl}Tie2cre and wild-type mice (FIG. 3h). Since pre-eclampsia is associated with placental and fetal growth restriction, we evaluated the effect of maternal endothelial cell BH4 deficiency on placental size and fetal weight at gestational day 18.5. We found that both fetuses and placentas were significantly smaller (˜10% for both fetuses and placentas) from pregnant Gch1^{fl/fl}Tie2cre females compared to wild-type females (FIGS. 3i and j). There was a corresponding reduction in crown-to-rump length of fetuses from pregnant Gch1^{fl/fl}Tie2cre mice (FIG. 3k). In keeping with the reduction in fetal size at E18.5, mean body weight of offspring born at term from Gch1^{fl/fl}Tie2cre females was significantly lower than that of offspring from wild-type mice (FIG. 3I and m). To distinguish the role of maternal endothelial cell BH4 biosynthesis from that of fetal BH4, wild-type females were mated with Gch1^{fl/fl}Tie2cre males, and Gch1^{fl/fl}Tie2cre females were mated with wild-type males, in order to generate pregnant female mice with matched litters with equal proportions of wild-type and Gch1^{fl/fl}Tie2cre offspring (for breeding strategy, see FIG. 19). Only Gch1^{fl/fl}Tie2cre females developed progressive hypertension during pregnancy, whereas wild-type females mice mated with Gch1^{fl/fl}Tie2cre males had normal blood pressure during pregnancy (FIG. 19). Similarly, the reduction in fetal size was dependent solely on maternal Gch1^{fl/fl}Tie2cre genotype. Furthermore, there was no difference in the reduction in fetal weight between male and female fetuses from Gch1^{fl/fl}Tie2cre mice (FIG. 20). We also evaluated the effect of heterozygous loss of Gch1 in maternal endothelial cells on fetal growth, in pregnant Gch1^{fl/+}Tie2cre mice, crossed with WT male mice to generate litters with only WT and Gch1^{fl/+}Tie2cre fetuses (i.e. no fetuses with homozygous deletion of endothelial cell Gch1). Pups born to pregnant Gch1^{fl/+}Tie2cre mice were significantly smaller than pubs born to WT mice (FIG. 17). These findings show that lack of maternal but not fetal endothelial cell BH4, causes pregnancy-induced hypertension and is responsible for reduced fetal growth.

### 2.4 Endothelial Cell BH4 is Required for Functional and Structural Uteroplacental Remodeling in Pregnancy

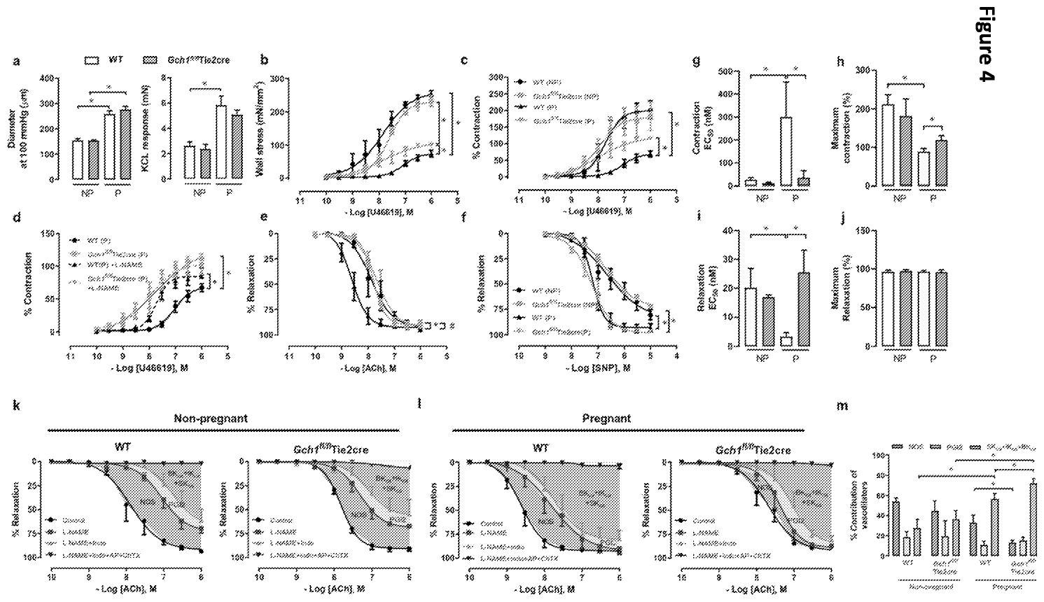

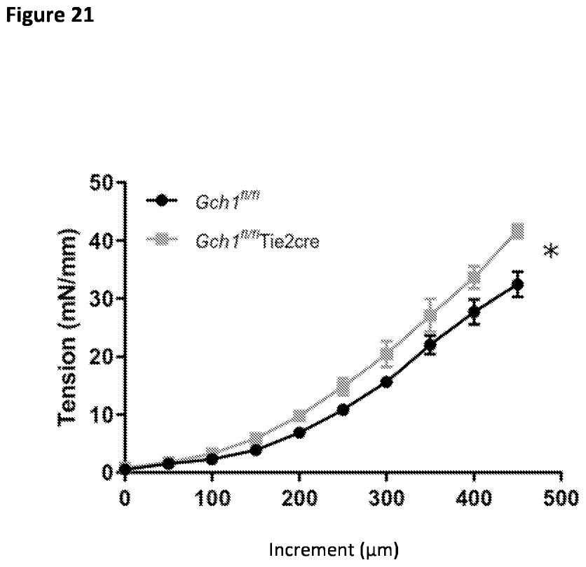

We next tested the effect of endothelial cell BH4 deficiency on vascular function in uterine arteries (UA) from non-pregnant mice, and from pregnant mice at E18.5, using wire myography. The maximal tension developed by uterine arteries from pregnant wild-type and Gch1^{fl/fl}Tie2cre mice were both significantly increased compared with non-pregnant mice (FIG. 4a), but wall stress (tension relative to vessel wall area) was reduced in pregnancy, reflecting pregnancy-associated changes in uterine artery endothelial function (FIG. 4b). However, the length-tension relationship of uterine arteries from pregnant Gch1^{fl/fl}Tie2cre mice was significantly altered compared with WT mice (FIG. 21). Furthermore, the vasoconstriction response to U46619 was increased (to levels observed in non-pregnant uterine arteries) in the presence of nitric oxide inhibitor, L-NAME in uterine arteries of wild-type mice but unchanged in Gch1^{fl/fl}Tie2cre mice (FIGS. 4c and d), indicating that eNOS-derived modulation of the constrictor response in wild-type uterine arteries is absent in pregnant Gch1f/fTie2cre mice.

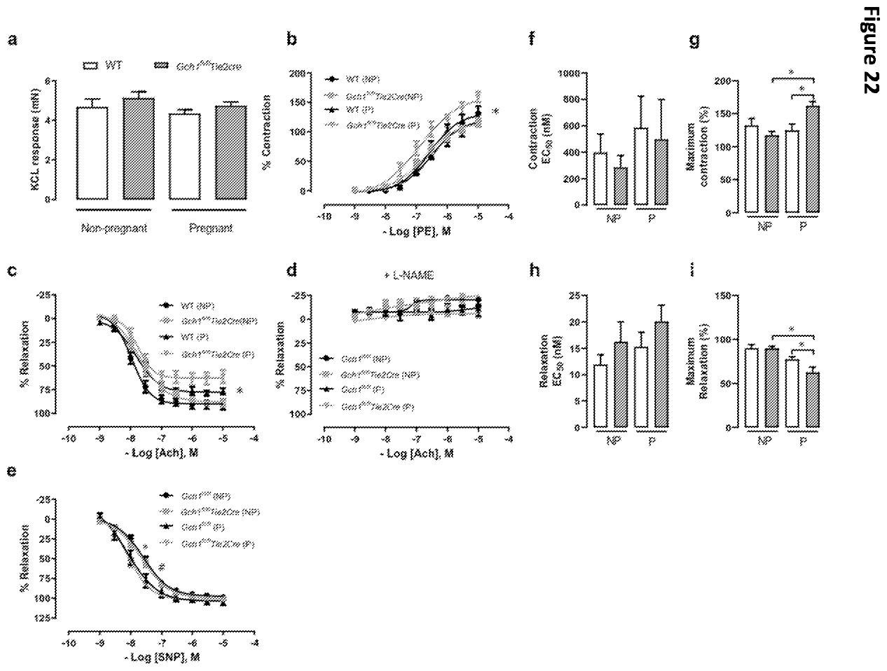

Endothelium-dependent vasodilatations to acetylcholine (ACh) were reduced in Gch1^{fl/fl}Tie2cre uterine arteries (FIG. 4E), with a corresponding increase in the EC50, whereas there was no difference in endothelium-independent vasodilatations to the direct NO donor, sodium nitroprusside SNP (FIG. 4e-j). Corresponding differences were observed in the constrictor responses of aortas to phenylephrine, and relaxation responses to ACh (FIG. 22). In contrast to wild-type uterine arteries, the NOS inhibitor L-NAME did not significantly inhibit ACh vasorelaxations in Gch1^{fl/fl}Tie2cre uterine arteries (FIGS. 4g and h), adding further evidence of the loss of eNOS-mediated vasodilator function in Gch1^{fl/fl}Tie2cre uterine arteries during pregnancy.

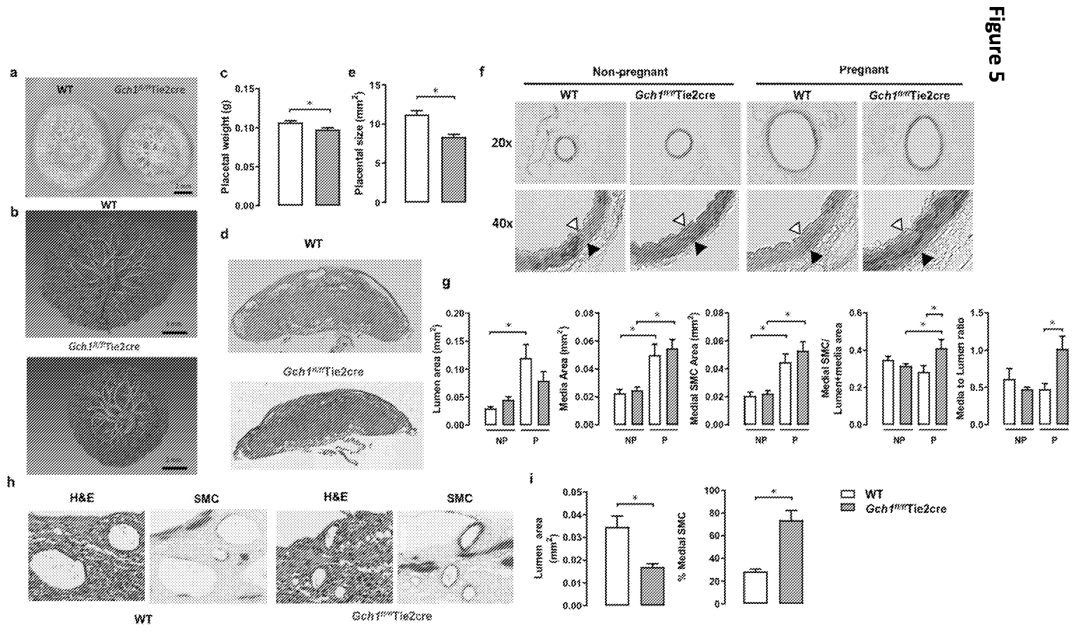

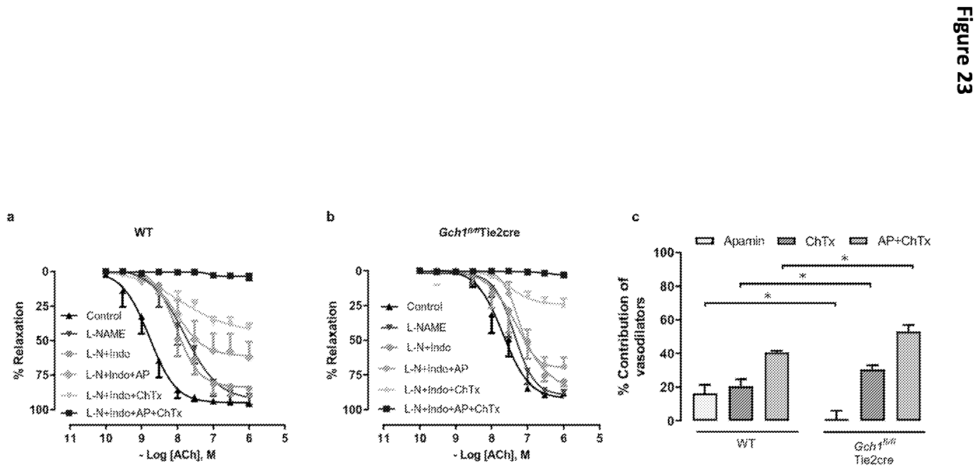

In order to investigate the alternative vasodilator mechanisms in Gch1^{fl/fl}Tie2cre uterine arteries during pregnancy, we next compared the inhibitory effects of L-NAME, indomethacin (an inhibitor of prostacyclin production), apamin (an inhibitor of small-conductance Ca2+-activated K+ channels) and charybdotoxin (an inhibitor of intermediate and large-conductance Ca2+-activated K+ channels). Addition of indomethacin had minimal effect on endothelium-dependent vasodilation in uterine arteries from either pregnant or nonpregnant wild-type and Gch1^{fl/fl}Tie2cre mice (FIG. 4kand l). In contrast, addition of apamin and charybdotoxin totally inhibited endothelium-dependent vasodilation in uterine arteries. Systematic quantification of the relative contributions of vasodilator responses in uterine arteries revealed a striking loss of L-NAME inhibited effects, and a significant increase in apamin/charybdotoxin inhibited effects in pregnant Gch1^{fl/fl}Tie2cre mice (FIG. 4m and FIG. 23). Taken together, these observations indicate that eNOS-derived vasodilator functions are lost in uterine arteries from pregnant Gch1^{fl/fl}Tie2cre mice, with Ca2+-activated K+ channels providing alternative endothelial-derived vasodilator functions. To determine the effects of endothelial cell BH4 deficiency on structural vascular remodelling in pregnancy, we compared uterine arteries and placental spiral arteries from wild-type and Gch1^{fl/fl}Tie2cre mice. Placental weights and placental size from Gch1^{fl/fl}Tie2cre pregnancies at gestational day E18.5 day were significantly reduced compared to those from wild-type pregnant females (FIG. 5a-e). In uterine arteries, pregnancy caused the expected marked increase in medial area, luminal diameter and luminal area in uterine arteries of both wildtype and Gch1^{fl/fl}Tie2cre mice compared to non-pregnant mice (FIGS. 5f and g), reflecting the increase in uterine blood flow during pregnancy. However, these changes were significantly impaired in uterine arteries of Gch1^{fl/+}Tie2cre mice (FIGS. 5f and g), with a smaller increase in luminal area, increased medial area and the area of vascular smooth muscle actin immunostaining, and a significant increase in media to lumen ratio. Furthermore, we found that decidual spiral arteries in placentas from Gch1^{fl/fl}Tie2cre mice failed to undergo the remodeling observed in wild-type mice, as indicated by reduced luminal area, and by increased muscularisation, revealed by immunohistochemistry for VSMC alpha-actin (FIGS. 5h and i). These findings demonstrate that selective endothelial cell BH4 deficiency causes impaired vascular remodelling in the maternal physiological response to pregnancy, in both uterine conduit arteries and placental resistance vessels.

2.5 Supplementation with BH4 Fails to Prevent Pregnancy-Induced Hypertension and Fetal Growth Restriction in Gch1^{fl/fl}Tie2cre Mice, but is Rescued by the Reduced Folate, 5-MTHF

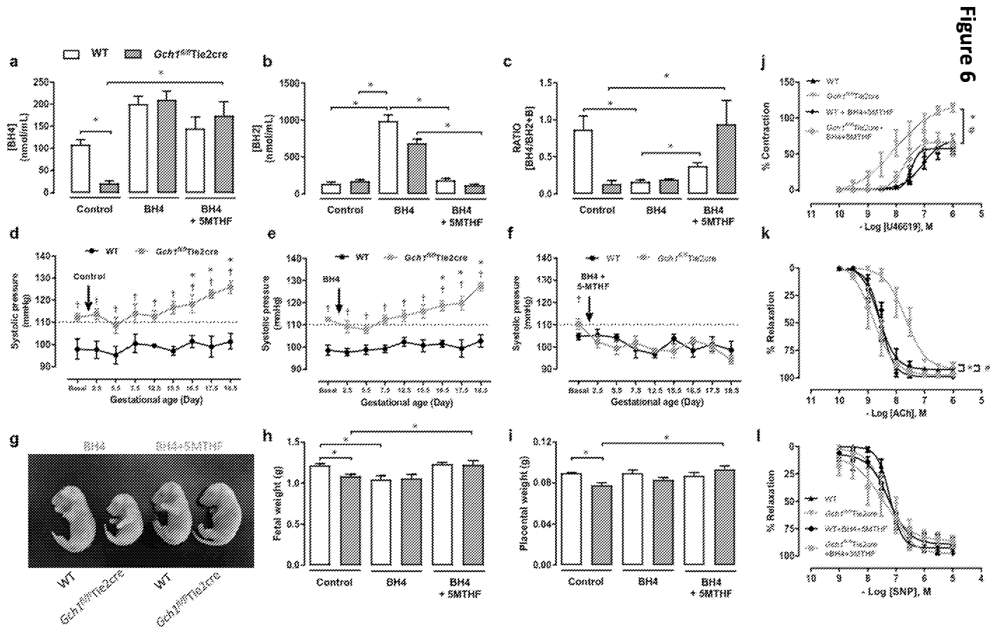

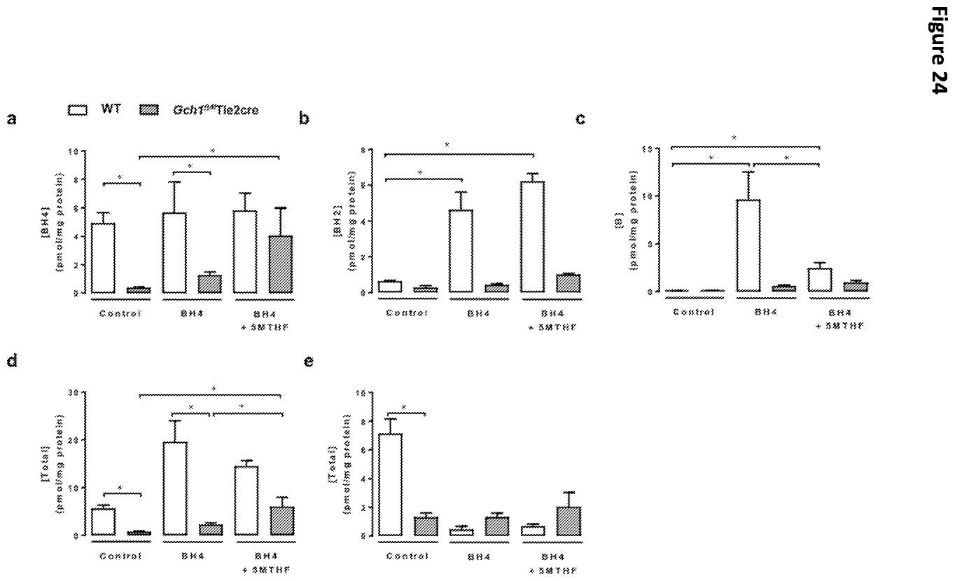

We reasoned that the consequences of endothelial cell BH4 deficiency in pregnancy might be prevented by BH4 supplementation, and that this may have translational therapeutic potential. We treated both Gch1^{fl/fl}Tie2cre and wild-type mice with oral BH4 supplementation for 3 days before timed-matings, and throughout their subsequent pregnancies. We found that oral BH4 supplementation increased plasma BH4 levels, but was associated with similar or even larger increases in the oxidized species, dihydrobiopterin (BH2), with a corresponding reduction in the BH4/BH2+B (total biopterins) ratio (FIG. 6a-c and FIG. 24), as we have previously observed in patients with established vascular disease¹⁰. There were no beneficial effects of oral BH4 supplementation in Gch1^{fl/fl}Tie2cre mice on either blood pressure or fetal growth restriction (FIG. 6d-i).

The enzyme dihydrofolate reductase (DHFR) reduces dihydrofolate to the fully reduced folate, tetrahydrofolate, and can also reduce oxidized BH2 to regenerate BH4. Accordingly, we hypothesized that co-administration of the fully reduced folate, 5-methyl-tetrahydrofolate (5-MTHF) may augment vascular BH4 levels (18). Treatment of pregnant mice with BH4 and 5-MTHF led to a striking restoration of BH4 levels in Gch1^{fl/fl}Tie2cre mice, without a significant elevation of BH2, and prevented the increased blood pressure and fetal growth restriction observed in untreated Gch1^{fl/fl}Tie2cre mice (FIG. 6d-i). In addition, we found that the combination of BH4+5-MTHF resulted in a striking normalization of both constriction responses to U46619 and relaxation responses to ACh, in Gch1^{fl/fl}Tie2cre mice, restoring the responses to those observed in wild-type animals (FIG. 6j-1).

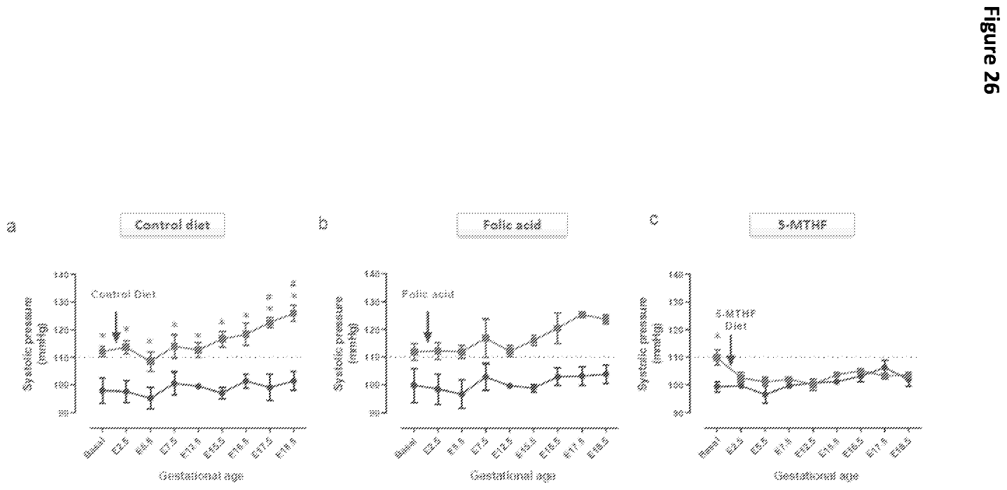

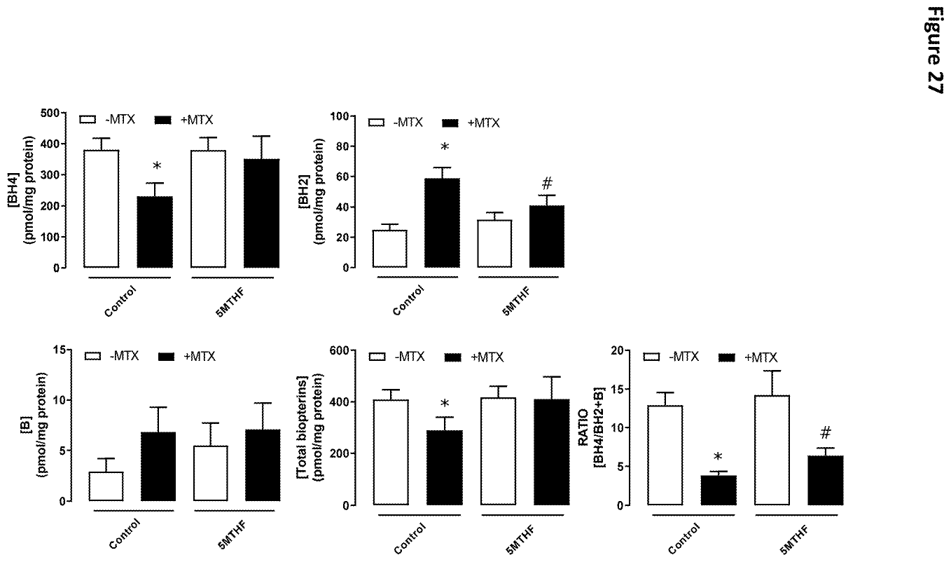

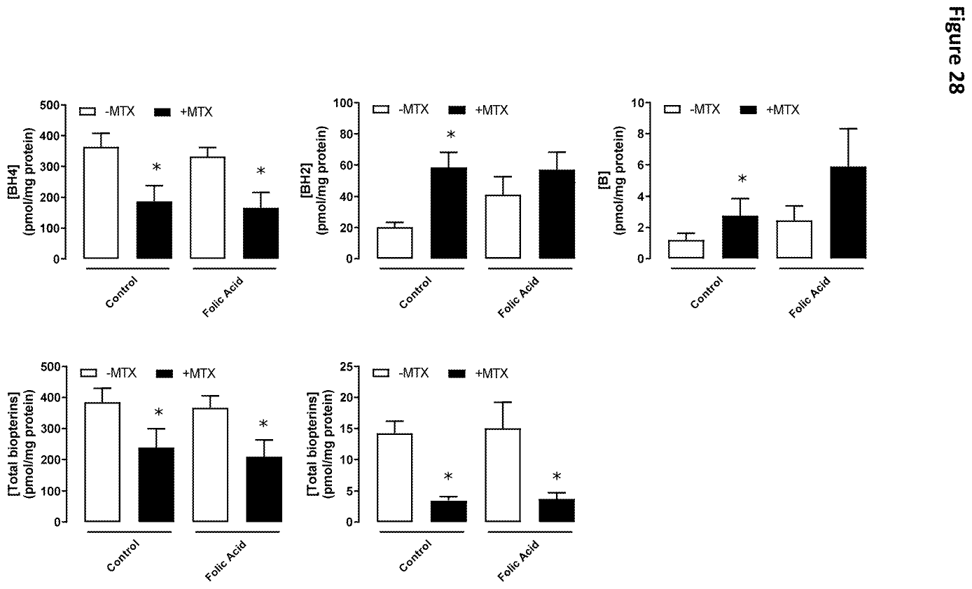

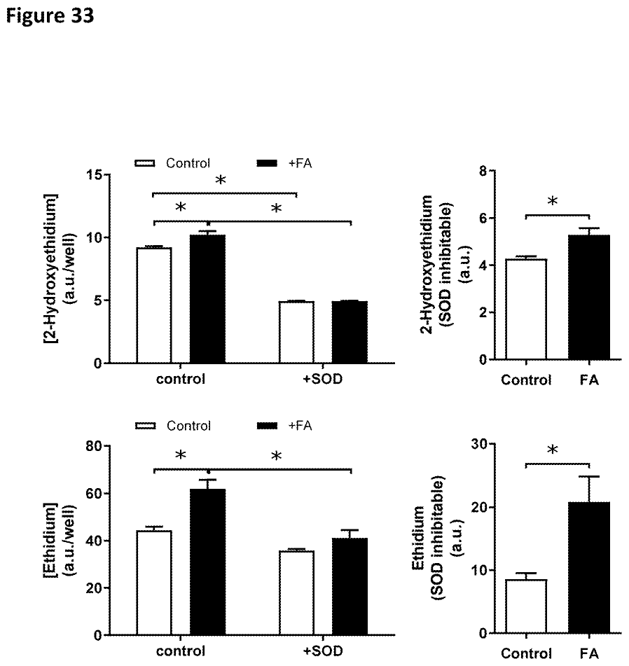

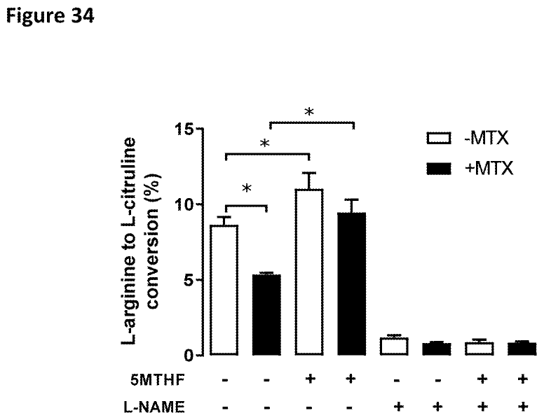

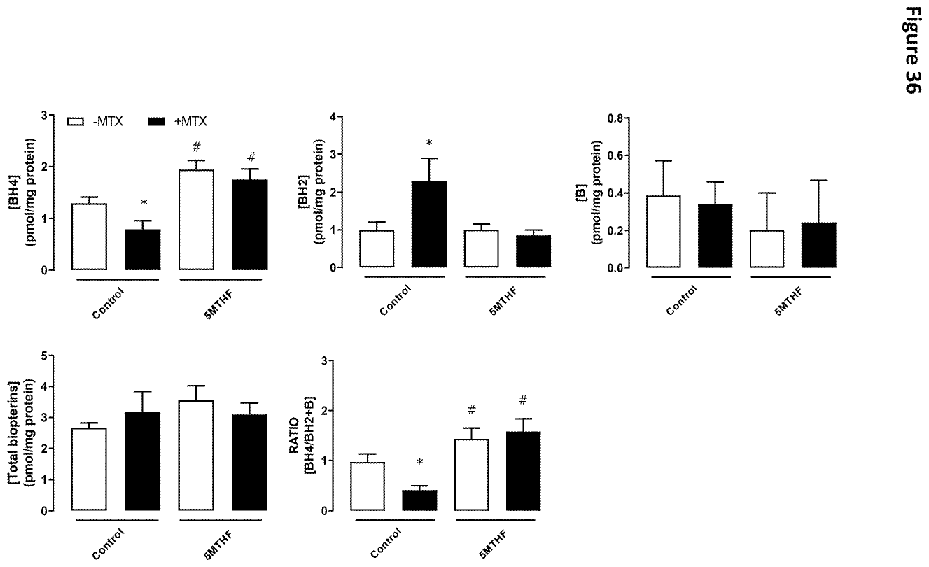

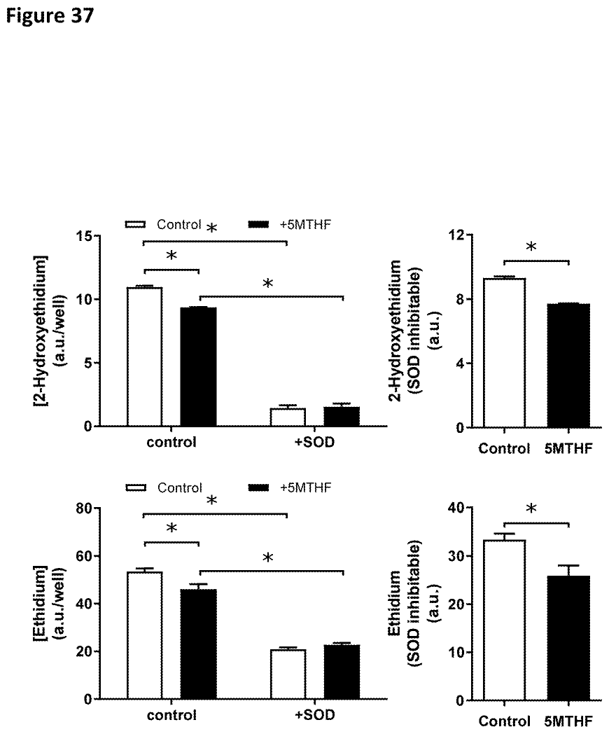

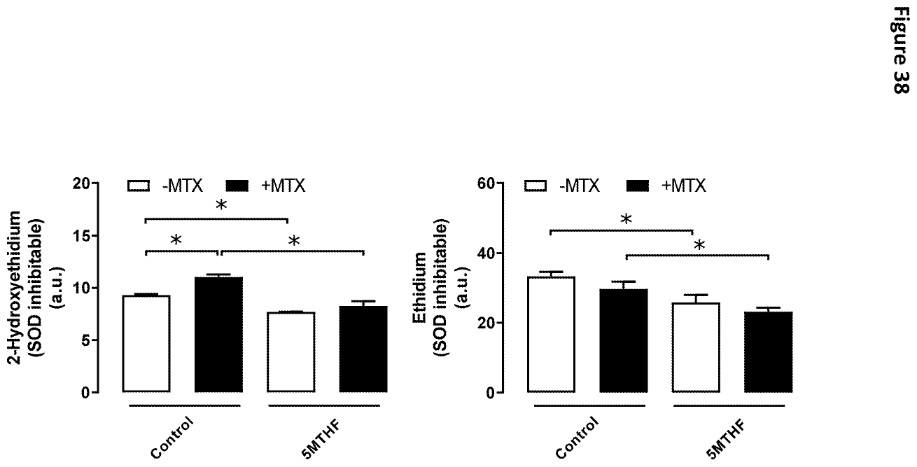

Furthermore, treatment with 5-MTHF alone also had therapeutic benefit. Treatment with 5-MTHF alone led to a striking and almost immediate decrease in systolic blood pressure of Gch1^{fl/fl}Tie2cre mice down to the normal blood pressures of wild type mice (see FIG. 26c). In addition, we found that treatment with the common supplement folic acid had no beneficial effect on systolic blood pressure, in fact the blood pressure increased over pregnancy in a similar manner to the control (see FIGS. 26a and 26b). In endothelial cells, incubation with folic acid had no effect on BH4 levels (FIG. 28), in contrast to the increase or restoration of endothelial cell BH4 levels by 5-MTHF in HUVECs from pregnancies with high blood pressure (FIG. 36) or in cells after incubation with methotrexate, an inhibitor of dihydrofolate reductase (DHFR) (FIG. 27). Indeed, folic acid increased the generation of reactive oxygen species (ROS) by endothelial cells (FIG. 33), whereas 5-MTHF decreased endothelial cell ROS production (FIGS. 37 and 38). 5-MTHF, but not folic acid, increased the activity of endothelial nitric oxide synthase (eNOS) in endothelial cells, as measured by L-arginine to L-citrulline conversion, that was inhibited by the eNOS inhibitor, L-NAME (FIG. 34). These observations indicate that the therapeutic effect discovered by the present inventors and demonstrated herein is not a ‘folate’ effect (i.e. well known classical effects of folates on methylation processes, homocysteine etc.) but is a new effect that is dependent on a chemically reduced form of folate (i.e. 5-MTHF), not an oxidised form (i.e. folic acid).

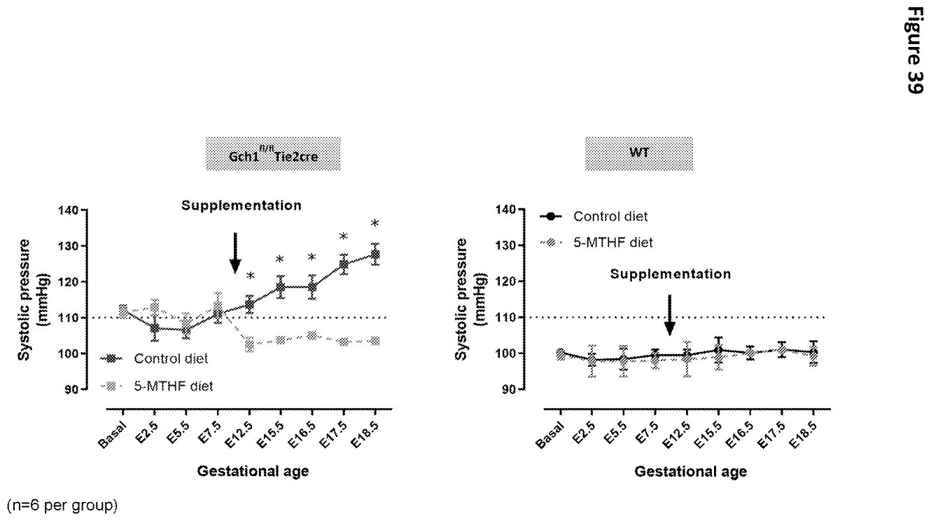

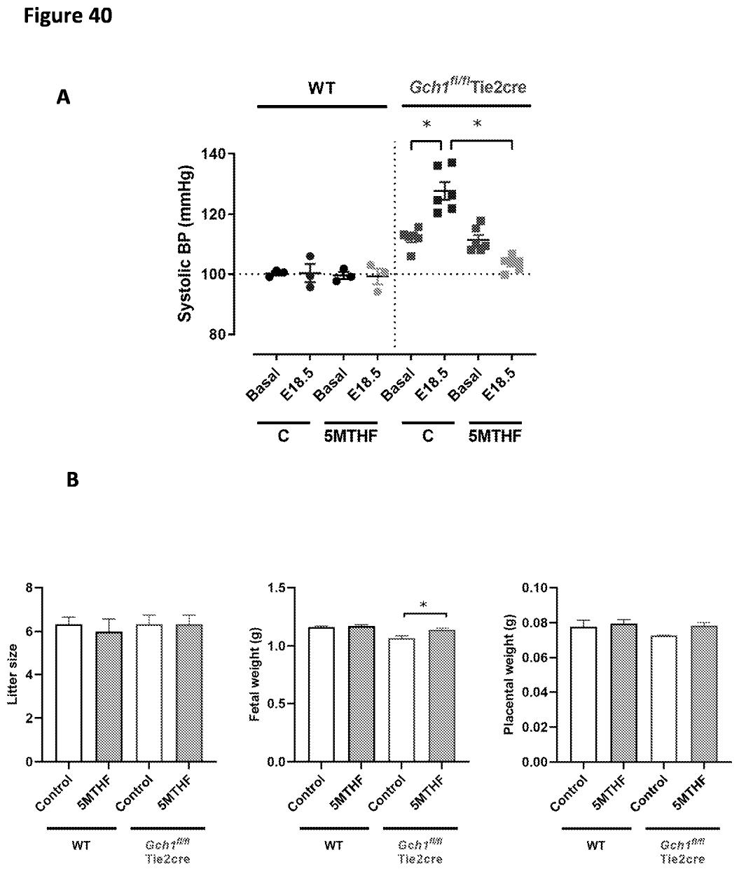

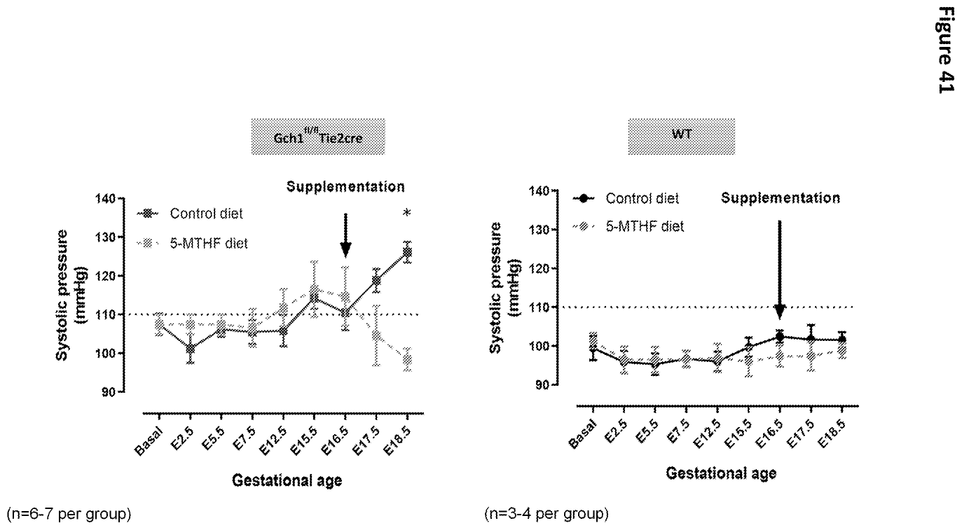

We further tested the ability of 5-MTHF treatment to lower blood pressure in pregnant Gch1^{fl/fl}Tie2cre mice, when 5-MTHF treatment was begun during, rather than before, pregnancy. We treated both Gch1^{fl/fl}Tie2cre and wild-type mice with oral 5-MTHF beginning at either 10.5 or 16.5 days after timed-matings, and throughout the remainder of the pregnancy, representing time points corresponding to mid- and late-pregnancy, respectively. We found that oral 5-MTHF treatment normalized the high blood pressure in Gch1^{fl/fl}Tie2cre mice when begun at day 10.5 (FIGS. 39 and 40), and significantly improved the fetal growth restriction (FIG. 40). 5-MTHF also normalized the high blood pressure in Gch1^{fl/fl}Tie2cre mice when begun at day 16.5 (FIG. 41). In both cases, the reduction in blood pressure following 5-MTHF treatment was observed rapidly, within 2 days of the initiation of treatment. These studies indicate that 5-MTHF is not only able to prevent the development of high blood pressure in pregnant Gch1^{fl/fl}Tie2cre mice, but is also able to treat high blood pressure that has already developed during pregnancy.

### REFERENCES

1. Aye C Y L, Lewandowski A J, Lamata P, Upton R, Davis E, Ohuma E O, Kenworthy Y, Boardman H, Wopperer S, Packham A, Adwani S, McCormick K, Papageorghiou A T and Leeson P. Disproportionate cardiac hypertrophy during early postnatal development in infants born preterm. Pediatr Res. 2017; 82:36-46.
2. Yu G Z, Aye C Y L, Lewandowski A J, Davis E F, Khoo C P, Newton L, Yang C T, Zen A A H, Simpson L J, O'Brien K, Cook D A, Granne 1, Kyriakou T, Channon K M, Watt S M and Leeson P. Association of Maternal Antiangiogenic Profile at Birth With Early Postnatal Loss of Microvascular Density in Offspring of Hypertensive Pregnancies. Hypertension. 2016; 68:749-759.
3. Yu G Z, Reilly S, Lewandowski A J, Aye C Y L, Simpson L J, Newton L, Davis E F, Zhu S J, Fox W R, Goel A, Watkins H, Channon K M, Watt S M, Kyriakou T and Leeson P.

Neonatal Micro-RNA Profile Determines Endothelial Function in Offspring of Hypertensive Pregnancies. Hypertension (Dallas, Tex: 1979). 2018; 72:937-945.
4. Motta-Mejia C, Kandzija N, Zhang W, Mhlomi V, Cerdeira A S, Burdujan A, Tannetta D, Dragovic R, Sargent I L, Redman C W, Kishore U and Vatish M. Placental Vesicles Carry Active Endothelial Nitric Oxide Synthase and Their Activity is Reduced in Preeclampsia. Hypertension. 2017; 70:372-+.
5. Dragovic R A, Collett G P, Hole P, Ferguson D J, Redman C W, Sargent I L and Tannetta D S. Isolation of syncytiotrophoblast microvesicles and exosomes and their characterisation by multicolour flow cytometry and fluorescence Nanoparticle Tracking Analysis. Methods. 2015; 87:64-74.
6. Chuaiphichai S, McNeill E, Douglas G, Crabtree M J, Bendall J K, Hale A B, Alp N J and Channon K M. Cell-autonomous role of endothelial GTP cyclohydrolase 1 and tetrahydrobiopterin in blood pressure regulation. Hypertension. 2014; 64:530-40.
7. Crabtree M J, Tatham A L, Al-Wakeel Y, Warrick N, Hale A B, Cai S, Channon K M and Alp N J. Quantitative regulation of intracellular endothelial nitric oxide synthase (eNOS) coupling by both tetrahydrobiopterin-eNOS stoichiometry and biopterin redox status: Insights from cells with tet-regulated GTP cyclohydrolase I expression. J Biol Chem. 2009; 284:1136-44.
8. Crabtree M J, Hale A B and Channon K M. Dihydrofolate reductase protects endothelial nitric oxide synthase from uncoupling in tetrahydrobiopterin deficiency. Free radical biology & medicine. 2011; 50:1639-46.
9. de Bono J P, Warrick N, Bendall J K, Channon K M and Alp N J. Radiochemical HPLC detection of arginine metabolism: measurement of nitric oxide synthesis and arginase activity in vascular tissue. Nitric Oxide. 2007; 16:1-9.
10. Cunnington C, Van Assche T, Shirodaria C, Kylintireas I, Lindsay A C, Lee J M, Antoniades C, Margaritis M, Lee R, Cerrato R, Crabtree M J, Francis J M, Sayeed R, Ratnatunga C, Pillai R, Choudhury R P, Neubauer S and Channon K M. Systemic and vascular oxidation limits the efficacy of oral tetrahydrobiopterin treatment in patients with coronary artery disease. Circulation. 2012; 125:1356-66.

1. A method of preventing or treating a disorder associated with low BH4 bioavailability in a subject, the method comprising administering to the subject a reduced folate;
wherein the disorder associated with low BH4 bioavailability is a pregnancy-related disorder; and
wherein the reduced folate is the sole active pharmaceutical agent.

2.-5. (canceled)

6. A method of preventing or treating a disorder associated with low BH4 bioavailability in a subject, the method comprising administering to said subject a composition comprising one or more active pharmaceutical ingredients,
wherein the disorder associated with low BH4 bioavailability is a pregnancy-related disorder, and
wherein the active pharmaceutical ingredient(s) consists of a reduced folate.

7. (canceled)

8. The method according to claim 1, wherein the reduced folate is in its natural stereoisomeric form.

9. The method according to claim 1, wherein the reduced folate increases or restores BH4 levels and/or wherein the reduced folate prevents oxidation of BH4.

10. (canceled)

11. The method according to claim 1, wherein the reduced folate is selected from the group consisting of:
DHF; 5-FTHF; THF; 5,10-CH-THF; 5,10-CH₂-THF, 5-MTHF, and 10-FTHF, and a pharmaceutically acceptable salt thereof, preferably wherein the reduced folate is 5-MTHF or a pharmaceutically acceptable salt thereof.

12-13. (canceled)

14. The method according to claim 1, wherein the disorder associated with low BH4 bioavailability is caused by impaired activity of one or more enzymes within the pterin biosynthetic pathway, optionally wherein the one or more enzymes within the pterin biosynthetic pathway are selected from:
GTP cyclohydrolase I (GTPCH), 6-pyruvoyl-tetrahydropterin synthase (PTPS), sepiapterin reductase (SP), dihydrofolate reductase (DHFR), dihydropteridine reductase (DHPR), pterin-4a-carbinolamine dehydratase (PCD), and endothelial NOS (eNOS).

15-18. (canceled)

19. The method according to claim 1, wherein the disorder associated with low BH4 bioavailability is a pregnancy-related vascular disorder, hypertension in pregnancy, or pre-eclampsia.

20-21. (canceled)

22. The method according to claim 1, wherein the reduced folate is for administration to a subject after having been diagnosed with a pregnancy-related vascular disorder.

23. The method according to claim 1, wherein the reduced folate, combination or composition is effective in the prevention of a disorder associated with insufficient BH4 levels in subsequent generations.

24. The method according to claim 1, wherein the prevention or treatment is in a subject who has previously suffered with a disorder associated with insufficient BH4 levels.

25. The method according to claim 1, wherein the prevention or treatment is in a subject who has previously suffered with hypertension in pregnancy or is in a subject who has previously suffered with pre-eclampsia.

26. (canceled)

27. The method according to claim 1, wherein the reduced folate is for administration at a dosage of between 1 mg and 50 mg per day, preferably between 2 mg and 30 mg per day, preferably between 3 mg and 25 mg per day, preferably between 4 mg and 20 mg per day, preferably between 5 mg and 15 mg per day.

28. The method according to claim 1, wherein the reduced folate is administered once or twice daily.

29. A method of selecting a subject that may benefit from treatment with a reduced folate and optionally BH4, a precursor or functional equivalent thereof, the method comprising determining the level of one or more markers of a disorder associated with low BH4 bioavailability in a subject, comparing the level(s) of the or each marker to a reference level(s) in a healthy subject, and selecting the subject for treatment if the subject exhibits an abnormal level of the one or more markers of a disorder associated with low BH4 bioavailability compared to the reference level.

30. The method according to claim 29, wherein the method further comprises providing a treatment to the selected subject.

31. The method according to claim 29, wherein the treatment comprises a reduced folate and optionally BH4, a precursor, or functional equivalent thereof.

32. The method according to claim 29, wherein the reduced folate is in its natural stereoisomeric form.

33. The method according to claim 31, wherein the reduced folate is provided to the selected subject before gestation, in the first trimester of pregnancy, in the second or third trimester of pregnancy, and/or after having measured an abnormal level of one or more markers of a disorder associated with low BH4 bioavailability in the subject.

34-38. (canceled)

39. A method of preventing oxidation of BH4, a precursor, or a functional equivalent thereof, in vitro, by using a reduced folate.

40. The method of claim 39, wherein the reduced folate is in its natural stereoisomeric form.

41. The method according to claim 6, wherein the reduced folate is in its natural stereoisomeric form.

42. The method according to claim 6, wherein the reduced folate increases or restores BH4 levels and/or wherein the reduced folate prevents oxidation of BH4.

43. The method according to claim 6, wherein the reduced folate is selected from the group consisting of:
DHF; 5-FTHF; THF; 5,10-CH-THF; 5,10-CH₂-THF, 5-MTHF, and 10-FTHF, and a pharmaceutically acceptable salt thereof, preferably wherein the reduced folate is 5-MTHF or a pharmaceutically acceptable salt thereof.

44. The method according to claim 6, wherein the disorder associated with low BH4 bioavailability is caused by impaired activity of one or more enzymes within the pterin biosynthetic pathway, optionally wherein the one or more enzymes within the pterin biosynthetic pathway are selected from:
GTP cyclohydrolase I (GTPCH), 6-pyruvoyl-tetrahydropterin synthase (PTPS), sepiapterin reductase (SP), dihydrofolate reductase (DHFR), dihydropteridine reductase (DHPR), pterin-4a-carbinolamine dehydratase (PCD), and endothelial NOS (eNOS).

45. The method according to claim 6, wherein the disorder associated with low BH4 bioavailability is a pregnancy-related vascular disorder, hypertension in pregnancy, or pre-eclampsia.

46. The method according to claim 6, wherein the reduced folate is for administration to a subject after having been diagnosed with a pregnancy-related vascular disorder.

47. The method according to claim 6, wherein the reduced folate, combination or composition is effective in the prevention of a disorder associated with insufficient BH4 levels in subsequent generations.

48. The method according to claim 6, wherein the prevention or treatment is in a subject who has previously suffered with a disorder associated with insufficient BH4 levels.

49. The method according to claim 6, wherein the prevention or treatment is in a subject who has previously suffered with hypertension in pregnancy or is in a subject who has previously suffered with pre-eclampsia.

50. The method according to claim 6, wherein the reduced folate is for administration at a dosage of between 1 mg and 50 mg per day, preferably between 2 mg and 30 mg per day, preferably between 3 mg and 25 mg per day, preferably between 4 mg and 20 mg per day, preferably between 5 mg and 15 mg per day.

51. The method according to claim 6, wherein the reduced folate is administered once or twice daily.
